(19) United States
(12) Patent Application Publication
(10) Pub. No.: US 2025/0387463 A1
(43) Pub. Date: Dec. 25, 2025

(54) Recombinant Modified saRNA (VRP) and Vaccinia Virus Ankara (MVA) Prime-Boost Regimen

(71) Applicant: Bavarian Nordic A/S, Hellerup (DK)

(72) Inventors: Robin Steigerwald, Munich (DE); José Medina Echeverz, Munich (DE); Alexander Heiseke, Munich (DE); Sonia Wennier, Gauting (DE); Ariane Volkmann, Andechs (DE); Juergen Hausmann, Gundelfingen (DE)

(73) Assignee: Bavarian Nordic A/S, Hellerup (DK)

(21) Appl. No.: 18/879,765

(22) PCT Filed: Jun. 29, 2023
(86) PCT No.: PCT/EP2023/067805

§ 371 Date: Dec. 28, 2024

(30) Foreign Application Priority Data
Jun. 29, 2022 (EP) 22181968.3

(51) Int. Cl. A61K 39/12; A61K 39/00; A61P 37/04

(52) U.S. Cl. CPC A61K 39/12; A61P 37/04; A61K 2039/53

(57) ABSTRACT

The present invention provides compositions, vaccines and methods for inducing protective immunity against an immunogen in humans. The protective immune response is obtained by using a saRNA, in particular a VRP vector as prime and a MVA for boost. Specifically, the present invention relates to genetically engineered (recombinant) VRP and MVA vectors comprising at least one heterologous nucleotide sequence encoding an antigenic determinant of an infectious virus such as EBV.

### FIELD OF THE INVENTION

The present invention relates to methods and compositions for enhancing an immune response in a subject comprising a self-amplifying RNA (saRNA), in particular a recombinant modified alpha virus replicon (VRP) and a vaccinia virus Ankara-based (MVA) vaccine against an infectious disease such as EBV in a human subject. The present invention also relates to vaccination methods, in particular heterologous prime-boost vaccination regimes, employing two viral vector compositions. More particularly, the invention relates to a recombinant VRP and a recombinant MVA for use in a heterologous prime-boost vaccination regime. The invention also relates to products, methods and uses thereof, e.g., suitable to induce a protective immune response in a subject.

### BACKGROUND OF THE INVENTION

An effective vaccine usually requires more than one time immunization in the form of prime-boost. Traditionally the same vaccines are given multiple times as homologous boosts. New findings suggested that prime-boost can be done with different types of vaccines containing the same antigens. In many cases such heterologous prime-boost can be more immunogenic than homologous prime-boost. Heterologous prime-boost represents a new way of immunization and will stimulate better understanding on the immunological basis of vaccines.

It is not unusual that multiple immunizations are required for many vaccines to be successful. For pediatric population, up to five immunizations may be needed, as is the case for Diphtheria, Tetanus and Pertussis (DTP) vaccine, which is given three times during the first six months after birth, followed by a fourth dose in the second year of life, and a final boost between four and six years of age. Still, some of the vaccines need additional boosts even in adults who have already received the complete immunization series, for example, the Tetanus-diphtheria (Td) vaccine, for which a boost is recommended every 10 years throughout a person's lifespan. While it is not entirely clear why some vaccines require more immunizations than others, it is well accepted that multiple immunizations (i.e. “prime-boost”) are critical for even the most successful vaccines. This principle applies to live attenuate vaccines (e.g., oral polio vaccine), inactivated vaccines (e.g., hepatitis A vaccine), recombinant protein subunit vaccines (e.g., hepatitis B vaccine) and polysaccharide vaccines (e.g., Haemophilus influenzae type b vaccine). For these vaccines, the prime-boost is “homologous” because the same vaccines given in the earlier priming immunizations are used for subsequent boost immunizations.

Over the past decade, studies have shown that prime-boost immunizations can be given with unmatched vaccine delivery methods while using the same antigen, in a “heterologous” prime-boost format. The most interesting and unexpected finding is that, in many cases, heterologous prime-boost is more effective than the “homologous” prime-boost approach. The rapid progress of novel vaccination approaches, such as DNA vaccines and viral vector-based vaccines, has certainly further expanded the scope of heterologous prime-boost vaccination (Excler J L, Plotkin S. The prime-boost concept applied to HIV preventive vaccines. Aids. 1997; 11 (Suppl A): S127-S137; Ramshaw I A, Ramsay A J. The prime-boost strategy: exciting prospects for improved vaccination. Immunol Today. 2000; 21:163-165; Lu S. Combination DNA plus protein HIV vaccines. Springer Semin Immunopathol. 2006; 28:255-265.

A 1992 landmark Science report was among the first to employ the heterologous prime-boost immunization technique in a non-human primate model (Hu S L, Abrams K, Barber G N, Moran P, Zarling J M, Langlois A J, Kuller L, Morton W R, Benveniste R E. Protection of macaques against SIV infection by subunit vaccines of SIV envelope glycoprotein gp160. Science. 1992; 255:456-459. First major report on the use of heterologous prime-boost vaccination approach, in the context of AIDS vaccine development). In that study, Macaca fascicularis were first immunized with recombinant vaccinia virus expressing SIVmne gp160 antigen and then boosted with gp160 protein produced in baculovirus-infected cells. Animals were protected from intravenous challenge of SIVmne viruses and this became one of the most promising protection results in the early HIV vaccine development effort.

Shiu-Lok Hu, the lead scientist of the above study, and his collaborators demonstrated previously, in rodents, that priming with a live recombinant virus and boosting with a subunit recombinant protein was more effective than immunization by either immunogen alone (Hu S L, Klaniecki J, Dykers T, Sridhar P, Travis B M. Neutralizing antibodies against HIV-1 BRU and SF2 isolates generated in mice immunized with recombinant vaccinia virus expressing HIV-1 (BRU) envelope glycoproteins and boosted with homologous gp160. AIDS Res Hum Retroviruses. 1991; 7:615-620).

In a separate study, Girard et al. also reported a significant increase in antibody titers in a chimpanzee primed with recombinant vaccinia virus and boosted multiple times with a mixture of recombinant HIV-1 proteins or synthetic peptides (Girard M, Kieny M P, Pinter A, Barre-Sinoussi F, Nara P, Kolbe H, Kusumi K, Chaput A, Reinhart T, Muchmore E, et al. Immunization of chimpanzees confers protection against challenge with human immunodeficiency virus. Proc Natl Acad Sci USA. 1991; 88:542-546). Furthermore, around the same time, in what may be the first human testing of the heterologous prime-boost immunization, Daniel Zagury of the Pierre and Marie Curie University in Paris inoculated himself with a recombinant vaccinia virus containing the HIV-1 Env gene and later gave a boost using a recombinant Env protein (Zagury D, Bernard J, Cheynier R, Desportes I, Leonard R, Fouchard M, Reveil B, Ittele D, Lurhuma Z, Mbayo K, et al. A group specific anamnestic immune reaction against HIV-1 induced by a candidate vaccine against AIDS. Nature. 1988; 332:728-731). Early work in other non-HIV areas include small animal studies conducted by Eckhart Wimmer's group who used synthetic peptides and inactivated polio virus for prime-boost immunizations (Emini E A, Jameson B A, Wimmer E. Priming for and induction of anti-poliovirus neutralizing antibodies by synthetic peptides. Nature. 1983; 304:699-703).

Initial efforts in the use of a heterologous prime-boost immunization approach for HIV-1 vaccine development was based on the following rationale:

Recombinant envelope (Env) glycoproteins, while being able to elicit isolate specific neutralizing antibody responses, were unable to elicit cytotoxic T cell responses, and on the other hand, immunization with recombinant vaccinia expressing HIV-1 antigens could elicit good T cell responses but not high levels of protective antibodies. Therefore, combined immunization including both of these two types of vaccines may be more effective than either immunogen alone (Hu S L, Klaniecki J, Dykers T, Sridhar P, Travis B M. Neutralizing antibodies against HIV-1 BRU and SF2 isolates generated in mice immunized with recombinant vaccinia virus expressing HIV-1 (BRU) envelope glycoproteins and boosted with homologous gp160. AIDS Res Hum Retroviruses. 1991; 7:615-620).

This statement established a key principle for the use of heterologous prime-boost immunizations, i.e., to elicit both humoral and cell-mediated immune responses. Modern immunology has established that such a balanced immune response is important for protection not only against viral infections but also other types of pathogens. Traditional vaccines, particularly inactivated and subunit vaccines, are not very effective in eliciting T cell responses. This requirement is even more important for HIV vaccine development. An ideal HIV vaccine should be able to generate “sterilizing antibodies” to prevent the virus from establishing an infection that is more difficult to eliminate once HIV-1 is integrated into the genome of the host's peripheral blood mononuclear cells (PBMCs).

At the same time, T cell immune responses play a key role in controlling the scale of infection, which may affect the long-term mortality and morbidity of the host.

Over the past few years, the use of heterologous prime-boost approaches in vaccine research has gained significant momentum against a wide range of pathogens. Several features have become apparent for this trend.

First, it is common to use the heterologous prime-boost approach to address some of the most challenging vaccine development objectives including malaria and tuberculosis due to the failure of other vaccination approaches. The idea is to focus on certain critical antigens and to elicit high quality immune responses involving different subsets of T cell immune responses. A DNA prime-MVA boost vaccine encoding thrombospondin-related adhesion protein partially protected healthy malaria-naïve adults against Plasmodium falciparum sporozoite challenge (Dunachie S J, Walther M, Epstein J E, Keating S, Berthoud T, Andrews L, Andersen R F, Bejon P, Goonetilleke N, Poulton I, et al. A DNA prime-modified vaccinia virus ankara boost vaccine encoding thrombospondin-related adhesion protein but not circumsporozoite protein partially protects healthy malaria-naive adults against Plasmodium falciparum sporozoite challenge. Infect Immun. 2006; 74:5933-5942). This study also highlights the importance of antigen selection for immune protection, made clear by the fact that the same combination vaccination using circumsporozoite protein, instead of the thrombospondin-related adhesion protein, did not elicit such protection.

For tuberculosis vaccine development, qualitatively and quantitatively different cellular immune responses have been elicited in rhesus macaques receiving a recombinant Bacille Calmette-Guerin (BCG) prime followed by an adenovirus 35 vector boost that expressed a fusion protein composed of Ag85A, Ag85B and TB104 (Magalhaes I, Sizemore D R, Ahmed R K, Mueller S, Wehlin L, Scanga C, Weichold F, Schirru G, Pau M G, Goudsmit J, et al. rBCG induces strong antigen-specific T cell responses in rhesus macaques in a prime-boost setting with an adenovirus 35 tuberculosis vaccine vector. PLOS ONE. 2008; 3: e3790). Alternatively, BCG can be used as a boost following a DNA vaccine prime. In one study conducted in calves, DNA prime with Ag85B, MPT64 and MPT83 antigens followed by a BCG boost was able to elicit higher immune responses and better protection than BCG alone against Mycobacterium bovis challenge (Cai H, Yu D H, Hu X D, Li S X, Zhu Y X. A combined DNA vaccine-prime, BCG-boost strategy results in better protection against Mycobacterium bovis challenge. DNA Cell Biol. 2006; 25:438-447).

Second, a well-designed heterologus prime-boost approach can expand the scope of immune responses. When mice were primed with DNA vaccine expressing ESAT6 and later received the same antigen in the form of recombinant protein as boost, production of Th1-type cytokines was increased significantly, as was the lgG2 to IgG1 ratio (Wang Q M, Sun S H, Hu Z L, Yin M, Xiao C J, Zhang J C. Improved immunogenicity of a tuberculosis DNA vaccine encoding ESAT6 by DNA priming and protein boosting. Vaccine. 2004; 22:3622-3627). In another murine study, prime with a DNA vaccine, expressing the gD antigen of herpes simplex virus type 2 (HSV-2), which preferentially induces Th1 type cellular immune responses, and boost with recombinant gD protein, which mainly induces Th2 biased responses, led to significantly enhanced antibody, T cell proliferation, and Th1 cytokine production (Sin J I, Bagarazzi M, Pachuk C, Weiner D B. DNA priming-protein boosting enhances both antigen-specific antibody and Th1-type cellular immune responses in a murine herpes simplex virus-2 gD vaccine model. DNA Cell Biol. 1999; 18:771-779).

Third, the prime-boost vaccine approach can also improve the effectiveness of existing vaccines. One example is the use of DNA prime, which increased antibody response levels, in animals later receiving boost with inactivated rabies vaccines (Biswas S, Reddy G S, Srinivasan V A, Rangarajan P N. Preexposure efficacy of a novel combination DNA and inactivated rabies virus vaccine. Hum Gene Ther. 2001; 12:1917-1922). Similarly, DNA prime can increase the titer and longevity of hyperimmune sera in animals to be immunized with the recombinant PA antigen against anthrax (Herrmann J E, Wang S, Zhang C, Panchal R G, Bavari S, Lyons C R, Lovchik J A, Golding B, Shiloach J, Lu S. Passive immunotherapy of Bacillus anthracis pulmonary infection in mice with antisera produced by DNA immunization. Vaccine. 2006; 24:5872-5880). Adding a DNA prime, mice boosted with the licensed hepatitis B surface protein vaccine were able to produce stronger and more homogenous antibody responses in a study group when compared to groups only receiving recombinant protein alone. Higher IL-12 and IFN-γ secretion in splenocytes were also observed (Xiao-wen H, Shu-han S, Zhen-lin H, Jun L, Lei J, Feng-juan Z, Ya-nan Z, Ying-jun G. Augmented humoral and cellular immune responses of a hepatitis B DNA vaccine encoding HBsAg by protein boosting. Vaccine. 2005; 23:1649-1656).

Finally, the prime-boost approach can have important practical applications in addressing vaccines with broad public health impact. In an animal model naïve to influenza infection, it has been shown that a heterologous one-time DNA prime and one-time inactivated influenza vaccine boost was more immunogenic than twice administered homologous prime-boost using either DNA or inactivated influenza vaccine alone (Wang S, Parker C, Taaffe J, Solorzano A, Garcia-Sastre A, Lu S. Heterologous HA DNA vaccine prime—inactivated influenza vaccine boost is more effective than using DNA or inactivated vaccine alone in eliciting antibody responses against H1 or H3 serotype influenza viruses. Vaccine. 2008; 26:3626-3633). This finding can be very useful for preparation against pandemic avian influenza. One of the key issues facing the development of influenza vaccines is the limited capacity and long cycle needed to produce traditional influenza vaccines. Usually, two immunizations are needed for avian influenza vaccines. It is feasible that targeted populations can first receive an avian influenza DNA vaccine prime long before any unexpected pandemic attack, which will greatly reduce the amount of vaccine needed at the time of outbreak of pandemic flu. This approach can also be useful for other forms of influenza, including human and swine influenza viruses. Adding a new strain of vaccine to the current trivalent influenza vaccines will require significant additional resources and time. A polyvalent DNA prime can cover a wide range of future potential viral strains at much lower cost.

Similar to other novel vaccine forms, the heterologous prime-boost approaches have also been studied as potential treatments for cancer. Using a recently identified six-transmembrane epithelial antigen of the prostate (STEAP), a heterologous DNA prime and Venezuelan equine encephalitis virus-like replicon particles (VRP) boost was able to elicit better immune responses against STEAP, including INF-gamma, TNF-alpha, and IL-12, when compared to either vaccine modality alone. This vaccination regimen induced a modest but significant delay in growth of established, 31 day-old tumors in mice (Garcia-Hernandez Mde L, Gray A, Hubby B, Kast W M. In vivo effects of vaccination with six-transmembrane epithelial antigen of the prostate: a candidate antigen for treating prostate cancer. Cancer Res. 2007; 67:1344-1351).

A fundamental but still mysterious question is why the heterlogous prime-boost is more effective than homologous prime-boost even when the same vaccine components are used for each. One way to study this question is to determine the importance of order of administration of heterologous prime-boost vaccines. Using a Mycobacterium bovis model, it was demonstrated that the order of prime-boost vaccination of neonatal calves with BCG and DNA vaccine, encoding Hsp65, Hsp70 and Apa, was not critical for enhancing protection against bovine tuberculosis (Skinner M A, Wedlock D N, de Lisle G W, Cooke M M, Tascon R E, Ferraz J C, Lowrie D B, Vordermeier H M, Hewinson R G, Buddle B M. The order of prime-boost vaccination of neonatal calves with Mycobacterium bovis BCG and a DNA vaccine encoding mycobacterial proteins Hsp65, Hsp70, and Apa is not critical for enhancing protection against bovine tuberculosis. Infect Immun. 2005; 73:4441-4444). In a different model, with DNA prime-protein boost using murine HSV-2 gD antigen, it was clear that DNA priming is critical because a reversed protein prime-DNA boost regimen produced antibody levels similar to those following homologous protein-protein vaccination, and failed to further enhance Th cell proliferative responses or cytokine production (Sin J I, Bagarazzi M, Pachuk C, Weiner D B. DNA priming-protein boosting enhances both antigen-specific antibody and Th1-type cellular immune responses in a murine herpes simplex virus-2 gD vaccine model. DNA Cell Biol. 1999; 18:771-779). In an even more detailed analysis using hepatitis C E2 as a model antigen, it was found that DNA prime-adenoviral vector boost elicited the highest level of Th1 CD4+ T cell responses when compared to the reversed adenoviral prime-DNA boost or homologous prime-boost with the same vaccines. More interestingly, the DNA prime-adenoviral vector boost regimen, but none of the other three possible prime-boost combinations, elicited CTL responses against three E2-specific epitopes and one of them was immunodominant (Park S H, Yang S H, Lee C G, Youn J W, Chang J, Sung Y C. Efficient induction of T helper 1 CD4+ T-cell responses to hepatitis C virus core and E2 by a DNA prime-adenovirus boost. Vaccine. 2003; 21:4555-4564 . . . . The order of prime-boost with DNA and adenovirus vector vaccines is important for the induction of cell mediated immune responses against HCV E2 antigen).

In an extensive non-human primate study, presented at the 2008 AIDS Vaccine conference in Cape Town, South Africa by Dr. Shiu-lok Hu from University of Washington, Seattle, vaccinia viral vector or DNA prime, followed by protein boost, generated better antibody responses than boosting with DNA or various viral vector vaccines. These two heterologous prime-boost regimens, including a protein boost component, but not any of the other combinations, were able to elicit better neutralizing antibodies and sterilizing immunity against a high-dose intrarectal challenge by SHIV_{sf162.p4} in ˜40% of immunized animals, and protected animals against peripheral CD4+ T-cell depletion.

Some studies have shown that DNA prime was able to improve the avidity of antibody responses elicited by protein-based vaccines (Richmond J F, Lu S, Santoro J C, Weng J, Hu S L, Montefiori D C, Robinson H L. Studies of the neutralizing activity and avidity of anti-human immunodeficiency virus type 1 Env antibody elicited by DNA priming and protein boosting. J Virol. 1998; 72:9092-9100; Wang S, Arthos J, Lawrence J M, Van Ryk D, Mboudjeka I, Shen S, Chou T H, Montefiori D C, Lu S. Enhanced immunogenicity of gp120 protein when combined with recombinant DNA priming to generate antibodies that neutralize the JR-FL primary isolate of human immunodeficiency virus type 1. J Virol. 2005; 79:7933-7937). Because DNA vaccines produce antigens in vivo, priming with a DNA vaccine may elicit memory B cells that are specific to sensitive conformation domains of an antigen. In a rabbit study, the delivery of primary HIV-1 gp120 antigens using the DNA prime-protein boost approach, but not the recombinant gp120 protein alone vaccine, was able to elicit conformation dependent CD4 binding site antibodies which are potentially important for neutralizing HIV-1 Vaine (M, Wang S, Crooks E T, Jiang P, Montefiori D C, Binley J, Lu S. Improved induction of antibodies against key neutralizing epitopes by human immunodeficiency virus type 1 gp120 DNA prime-protein boost vaccination compared to gp120 protein-only vaccination. J Virol. 2008; 82:7369-7378. Including a DNA priming immunization was able to elicit conformation sensitive antibody responses when compared to protein alone HIV-1 Env vaccine).

The immunogenicity of heterologous prime-boost can be further improved by including other factors that may further facilitate or enhance the effect of vaccines. For example, including plasmid cytokines and colony-stimulating factors could enhance the immunogenicity of DNA prime-viral vector boosting HIV-1 vaccines (Barouch D H, Mckay P F, Sumida S M, Santra S, Jackson S S, Gorgone D A, Lifton M A, Chakrabarti B K, Xu L, Nabel G J, et al. Plasmid chemokines and colony-stimulating factors enhance the immunogenicity of DNA priming-viral vector boosting human immunodeficiency virus type 1 vaccines. J Virol. 2003; 77:8729-8735). The potency of DNA vaccine prime can be enhanced by using a micorparticle based formulation followed with a protein boost (Otten G R, Schaefer M, Doe B, Liu H, Srivastava I, Megede J, Kazzaz J, Lian Y, Singh M, Ugozzoli M, et al. Enhanced potency of plasmid DNA microparticle human immunodeficiency virus vaccines in rhesus macaques by using a priming-boosting regimen with recombinant proteins. J Virol. 2005; 79:8189-8200). However, it is not clear whether using different adjuvants for a protein vaccine as boost will make any difference.

Heterologous prime-boost vaccination, using both traditional and novel immunization approaches, provides exciting opportunities to elicit unique immune responses to allow for improved immunogenicity and/or protection. Research has shown that the heterologous prime-boost can take various forms and that the order of prime-boost administration may be important although this may be antigen-dependent and may be influenced by the host species and the type(s) of immune responses to be achieved. Future studies will need to focus more on the mechanisms behind the heterologous prime-boost vaccination approach and solve practical issues related to a two-component vaccine, including costs of vaccines and any currently unidentified issues of safety.

There is still an unmet need for improved vaccines that elicit immune responses against different viruses and that utilized the heterologous prime-boost vaccination regimen.

### BRIEF SUMMARY OF THE INVENTION

It is discovered in the present invention that various prime-boost combinations of replication incompetent vectors generate effective immune protection against an infectious disease, especially EBV infection.

Accordingly, one general aspect of the present invention relates to a vaccine combination comprising
(a) a first composition comprising an immunologically effective amount of a saRNA comprising a nucleic acid encoding an antigenic protein, together with a pharmaceutically acceptable carrier; and
(b) a second composition comprising an immunologically effective amount of an MVA vector comprising a nucleic acid encoding an antigenic protein, together with a pharmaceutically acceptable carrier;
   wherein one of the compositions is a priming composition and the other composition is a boosting composition.

In an additional aspect, the present invention relates to a kit comprising:
(a) a first composition comprising an immunologically effective amount of a saRNA comprising a nucleic acid encoding antigenic protein, together with a pharmaceutically acceptable carrier; and
(b) a second composition comprising an immunologically effective amount of a MVA vector comprising a nucleic acid encoding an antigenic protein, together with a pharmaceutically acceptable carrier;
   wherein one of the compositions is a priming composition and the other composition is a boosting composition.

In an additional aspect, the present invention relates to a method of inducing an immune response against a virus in a subject, the method comprising administering to the subject:
(a) a first composition comprising an immunologically effective amount of a saRNA comprising a nucleic acid encoding antigenic proteins, together with a pharmaceutically acceptable carrier; and
(b) a second composition comprising an immunologically effective amount of a MVA vector comprising a nucleic acid encoding an antigenic protein, together with a pharmaceutically acceptable carrier;
   wherein one of the compositions is a priming composition and the other composition is a boosting composition.

In certain embodiments, the first composition is used for priming an immune response and the second composition is used for boosting said immune response or vice versa.

In certain embodiments, the present invention relates to a recombinant Modified Vaccinia Virus (MVA) vector and a VRP vector comprising a nucleotide sequence encoding two or more antigenic determinants of a virus causing an infectious disease.

In a preferred embodiment, the antigenic protein is any of the structural and non-structural of EBV. In a preferred embodiment, the antigenic proteins are selected from gp350, gH, gL, EBNA3A, BRLF1/BZLF1 fusion.

In another embodiment, the VRP is VEEV TC83 and the MVA is MVA-BN.

In yet another embodiment, the VRP vector in the first composition comprises a nucleic acid encoding an antigenic protein selected from the group consisting of SEQ ID NO: 1, SEQ ID NO: 2, and SEQ ID NO: 3.

In yet another embodiment, the MVA vector in the second composition comprises a nucleic acid encoding antigenic proteins selected from the group consisting of SEQ ID NO: 1, SEQ ID NO: 2, SEQ ID NO: 3, SEQ ID NO: 5 and SEQ ID NO: 4.

In yet another aspect, the present invention relates to a vaccine combination comprising
(a) a first composition comprising an immunologically effective amount of a saRNA comprising a nucleic acid encoding an antigenic protein, together with a pharmaceutically acceptable carrier; and
(b) a second composition comprising an immunologically effective amount of an MVA vector comprising a nucleic acid encoding an antigenic protein, together with a pharmaceutically acceptable carrier;
   wherein one of the compositions is a priming composition and the other composition is a boosting composition.
      for use in generating a protective immune response against an infectious disease, wherein the first composition is used for priming said immune response and the second composition is used for boosting said immune response or vice versa.

The boosting composition may comprise two or more doses of the vector of the boosting composition.

In additional aspects, the present invention relates to the use of the vaccine combination or the kit comprising
(a) a first composition comprising an immunologically effective amount of a saRNA vector comprising a nucleic acid encoding antigenic protein, together with a pharmaceutically acceptable carrier; and
(b) a second composition comprising an immunologically effective amount of a MVA vector comprising a nucleic acid encoding an antigenic protein, together with a pharmaceutically acceptable carrier;
   wherein one of the compositions is a priming composition and the other composition is a boosting composition.
      for manufacturing a pharmaceutical composition or medicament for treatment and/or prevention of an infectious disease.

In yet an aspect, the present invention relates to a pharmaceutical composition comprising the vaccine combination comprising
(a) a first composition comprising an immunologically effective amount of a saRNA vector comprising a nucleic acid encoding antigenic protein, together with a pharmaceutically acceptable carrier; and
(b) a second composition comprising an immunologically effective amount of a MVA vector comprising a nucleic acid encoding an antigenic protein, together with a pharmaceutically acceptable carrier;
   wherein one of the compositions is a priming composition and the other composition is a boosting composition and a pharmaceutically acceptable carrier, diluent and/or additive.

These and other objects of the invention will be described in further detail in connection with the detailed description of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1A shows the structure and genetic organization of MVA-mBN443. FIG. 1B shows the structure and genetic organization of VRP-BN011. UTR=5′ or 3′ untranslated region of VEEV; nsp=coding region for the non-structural protein, SUBG PROM=subgenomic promoter of VEEV; EBV gp350 multimer: EBV gp350=Epstein Barr virus glycoprotein gp350 amino acids 2-434 with a C-terminal fusion of a GCN4 multimerization domain; T2A=2a peptide sequence; RKRR=furin cleavage site; P2A=2a peptide sequence; gH=glycoprotein H of EBV; gL=glycoprotein L of EBV

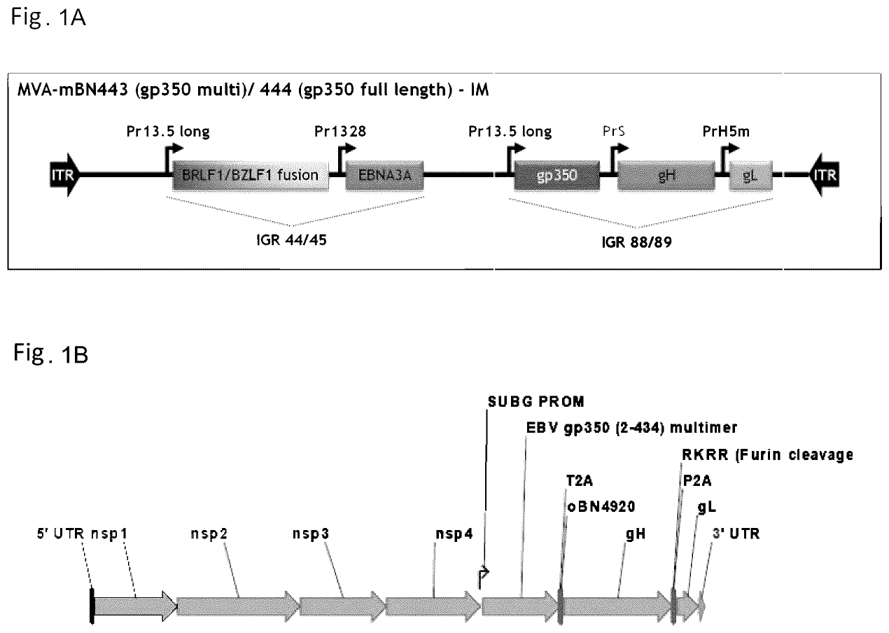

FIG. 2 shows the Gp350-specific IgG responses per group. Animals (n=3) were administered two times IM on Day 1 and 29 with the respective prime-boost regimen as outlined in the figure legend by the respective color code and described in Table 1. Blood was collected prior to administration (predose) and on Day 29 and 43. Serum was tested using multiplex ELISA. In this graph, the geometric mean and the geometric standard deviation of gp350-specific IgG concentrations are indicated as EU.

FIG. 3 shows Neutralizing antibodies per group. Animals (n=3) were administered two times IM on Day 1 and 29 with the respective prime-boost regimen as outlined in the figure legend by the respective color code and described in Table 1. Blood was collected prior to administration (predose) and on Day 29 and 43 and serum was tested using a flowcytometry-based neutralizing test. In this graph, the geometric mean and the geometric standard deviation are shown.

FIG. 4 shows Gp350-specific T cell responses per group. Animals (n=3) were administered two times IM on Day 1 and 29 with the respective prime-boost regimen as outlined in the figure legend by the respective color code and described in Table 1. Blood was collected prior to administration (predose) and on Day 8 and 36 and PBMC were tested using ELISPOT analysis. In this graph, mean of number of spot forming units (SFU) per 1×10⁶ PBMC and the standard error of mean (SEM) are shown.

FIG. 5 shows IFN-γ ELISPOT responses of splenocytes two weeks after the boost. Mice were immunized intramuscular (IM) with TNE (buffer control), MVA-EBV or VRP-EBV on day 0 and boosted on day 21 with the same test articles either homologous or heterologous. 2 weeks after the boost, splenocytes were isolated and re-stimulated in an ELISPOT assay with three gp350 peptides (EBV peptide #1 (MEAALLVCQYTIQSL); EBV gp350 peptide #25 (LGAGELALTMRSKKL) and EBV peptide #26 (ELALTMRSKKLPINV)). IFN-γ positive spots were counted. All counts are background subtracted (medium control stimulation). Bars represent mean±SEM;

FIG. 6 shows OVA-specific CD8 T cell responses in the blood five days after boost immunization. Mice were immunized subcutaneously (SC) with TNE (buffer control), MVA-OVA or VRP-OVA on day 0 and boosted on day 21 with the same test articles either homologous or heterologous. Five days after the boost, blood was drawn and OVA-specific CD8 T cells were determined by flow cytometry analysis using a SIINFEKL-dextramer. Bars represent mean±SEM. N=5 mice per group.

FIG. 7 shows OVA-specific CD8 T cell responses in the spleen four weeks after the boost immunization. Mice were immunized subcutaneously (SC) with TNE (buffer control), MVA-OVA or VRP-OVA on day 0 and boosted on day 21 with the same test articles either homologous or heterologous. Four weeks after the boost immunization splenocytes were isolated and restimulated with OVA257-264 peptide or OVA55-62 peptide for 6 h. Cytokine producing cells were detected by flow cytometry. Bars represent mean±SEM. N=5 mice per group.

FIG. 8 shows OVA-specific serum total IgG titers upon heterologous VRP/MVA immunization. Mice were immunized subcutaneously (SC) with TNE (buffer control), MVA-OVA or VRP-OVA on day 0 and boosted on day 21 with the same test articles either homologous or heterologous. On days 14 and 35 blood was drawn and serum was isolated. OVA-specific total IgG titers were determined by ELISA. Bars represent mean±SEM. N=5 mice per group.

FIG. 9 shows IFN-γ ELISPOT responses of splenocytes two weeks after the boost. Mice were immunized intramuscular (IM) with TNE (buffer control), MVA-EBV or SFV-VRP-EBV on day 0 and boosted on day 21 with the same test articles either homologous or heterologous. 2 weeks after the boost, splenocytes were isolated and re-stimulated in an ELISPOT assay with three gp350 peptides (EBV peptide #1 (MEAALLVCQYTIQSL); EBV gp350 peptide #25 (LGAGELALTMRSKKL) and EBV peptide #26 (ELALTMRSKKLPINV)). IFN-γ positive spots were counted. All counts are background subtracted (medium control stimulation). Bars represent mean±SEM;

### BRIEF DESCRIPTION OF SEQUENCES

SEQ ID NO: 1 depicts the nucleic acid sequence of gp350 multimer (1455 nucleotides).
SEQ ID NO: 2 depicts the nucleic acid sequence of gH (2121 nucleotides).
SEQ ID NO: 3 depicts a nucleic acid sequence of gL (414 nucleotides).
SEQ ID NO: 4 depicts the nucleic acid sequence of the fusion gene made from the sequences of BZLF1-BRLF1 (2283 nucleotides).
SEQ ID NO: 5 depicts the nucleic acid sequence of EBNA3A (2892 nucleotides).
SEQ ID NO: 6 depicts the DNA Sequence of one loxPV site.
SEQ ID NO: 7 depicts the nucleic acid sequence of the Pr13.5 long promoter.
SEQ ID NO: 8 depicts the nucleic acid sequence of the PrS promoter.
SEQ ID NO: 9 depicts the nucleic acid sequence of the PrH5m promoter.
SEQ ID NO: 10 depicts a nucleic acid sequence of Pr1328 promoter.
SEQ ID NO: 11 depicts a nucleic acid sequence of a 2A peptide (T2A).
SEQ ID NO: 12 depicts a nucleic acid sequence of 2A peptide (P2A).
SEQ ID NO: 13 depicts a nucleic acid sequence of the linker GCN4.

### DETAILED DESCRIPTION OF THE INVENTION

It could not have been expected from what is taught and what was achieved in the prior art that the heterologous prime-boost regimens with saRNA, in particular VRP, as prime vaccination and MVA as booster vaccination were highly immunogenic in terms of gp350-specific IgG and neutralizing antibodies while the homologous vaccination regimens with MVA or VRP, or the administration of Ad as booster vaccine had the least immunogenic effect.

The heterologous prime-boost regimen would generate an immune response that confers protection in non-human primates against a virus infection, in particular against EBV. Of course, from the data generated by the present inventors and their observations, it is more than reasonable and plausible to conclude that the vaccine regimen would also induce an immune response in humans and not only specifically against EBV but also other diseases caused by other disease associated antigens including an infectious disease antigen or a tumor-associated antigen. Indeed, the FDA accepts non-human primate models as proof that a vaccine which confers protection in these non-human primates is likewise suitable in humans.

Especially, the present inventors have also found that a vaccination regime comprising VRP as prime vaccination resulted in higher gH/gL/gp42-complex and gH-specific IgG responses than using MVA as prime vaccination. One vaccination with MVA or VRP of NHP with pre-existing immunity to gH/gL/gp42 or gH only boosted the gH/gL/gp42-complex and gH-specific IgG response, while a second administration had no or only little additive boost effect.

The invention thus provides vaccines or vaccine combinations for use in generating an immune response that confers protection against an infectious disease antigen or a tumor-associated antigen, e.g. by EBV and vaccines or vaccine combinations which can be used for manufacturing of a vaccine against said antigens.

### Definitions

Before the present invention is described in detail below, it is to be understood that this invention is not limited to the particular methodology, protocols and reagents described herein as these may vary. It is also to be understood that the terminology used herein is for the purpose of describing particular embodiments only and is not intended to limit the scope of the present invention which will be limited only by the appended claims. Unless defined otherwise, all technical and scientific terms used herein have the same meanings as commonly understood by one of ordinary skill in the art.

It must be noted that, as used herein, the singular forms “a”, “an”, and “the” include plural references unless the context clearly indicates otherwise. Thus, for example, reference to “a structural protein” includes one or more structural proteins and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain, using no more than routine experimentation, many equivalents to the specific embodiments of the invention described herein. Such equivalents are intended to be encompassed by the present invention.

The term “about” when used in connection with a numerical value is meant to encompass numerical values within a range having a lower limit that is 5% smaller than the indicated numerical value and having an upper limit that is 5% larger than the indicated numerical value unless the context clearly indicates otherwise.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. Any of the aforementioned terms (comprising, containing, including, having), whenever used herein in the context of an aspect or embodiment of the present invention may be substituted with the term “consisting of”, though less preferred.

When used herein “consisting of” excludes any element, step, or ingredient not specified in the claim element. When used herein, “consisting essentially of” does not exclude materials or steps that do not materially affect the basic and novel characteristics of the claim.

The term “antigen” includes all related epitopes of a particular compound, composition or substance. The term “epitope” or “antigenic determinant” refers to a site on an antigen to which B- and/or T-cells respond, either alone or in conjunction with another protein such as, for example, a major histocompatibility complex (“MHC”) protein or a T-cell receptor. Epitopes can be formed both from contiguous amino acids or noncontiguous amino acids juxtaposed by secondary and/or tertiary folding of a protein. Epitopes formed from contiguous amino acids are typically retained on exposure to denaturing solvents, while epitopes formed by tertiary folding are typically lost on treatment with denaturing solvents. An epitope typically includes at least 5, 6, 7, 8, 9, 10 or more amino acids—but generally less than 20 amino acids—in a unique spatial conformation. Methods of determining spatial conformation of epitopes include, for example, x-ray crystallography and 2-dimensional nuclear magnetic resonance. See, e.g., “Epitope Mapping Protocols” in Methods in Molecular Biology, Vol. 66, Glenn E. Morris, Ed (1996).

An antigen can be a tissue-specific (or tissue-associated) antigen or a disease-specific (or disease-associated) antigen. Those terms are not mutually exclusive, because a tissue-specific antigen can also be a disease-specific antigen. A tissue-specific antigen is expressed in a limited number of tissues. Tissue-specific antigens include, for example, prostate-specific antigen (“PSA”). A disease-specific antigen is expressed coincidentally with a disease process, where antigen expression correlates with or is predictive of development of a particular disease. Disease-specific antigens include, for example, HER-2, which is associated with certain types of breast cancer, or PSA, which is associated with prostate cancer. A disease-specific antigen can be an antigen recognized by T-cells or B-cells.

A malignant growth arising from a particular body tissue that has undergone characteristic loss of structural differentiation, generally accompanied by increased capacity for cell division, invasion of surrounding tissue, and the capacity for metastasis. Tumors may be benign or malignant. For example, prostate cancer is a malignant neoplasm that arises in or from prostate tissue, ovarian cancer is a malignant neoplasm that arises in or from ovarian tissue, colon cancer is a malignant neoplasm that arises in or from colon tissue, and lung cancer is a malignant neoplasm that arises in or from lung tissue. Residual cancer is cancer that remains in a subject after treatment given to the subject to reduce or eradicate the cancer. Metastatic cancer is a cancer at one or more sites in the body other than the site of origin of the original (primary) cancer from which the metastatic cancer is derived.

A “conservative” variant is a variant protein or polypeptide having one or more amino acid substitutions that do not substantially affect or decrease an activity or antigenicity of the protein or an antigenic epitope thereof. Generally conservative substitutions are those in which a particular amino acid is substituted with another amino acid having the same or similar chemical characteristics. For example, replacing a basic amino acid such as lysine with another basic amino acid such as arginine or glutamine is a conservative substitution. The term conservative variant also includes the use of a substituted amino acid in place of an unsubstituted parent amino acid, provided that antibodies raised to the substituted polypeptide also immunoreact with the unsubstituted polypeptide, and/or that the substituted polypeptide retains the function of the unsubstituted polypeptide. Non-conservative substitutions are those that replace a particular amino acid with one having different chemical characteristics, and typically reduce an activity or antigenicity of the protein or an antigenic epitope thereof.

Specific, non-limiting examples of conservative substitutions include the following examples:

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

“A disease-associated antigen” is expressed coincidentally with a particular disease process, where antigen expression correlates with or predicts development of that disease. Disease-associated antigens include, for example, HER-2, which is associated with certain types of breast cancer, or prostate-specific antigen (“PSA”), which is associated with prostate cancer. A disease-associated antigen can be an antigen recognized by T-cells or B-cells. Some disease-associated antigens may also be tissue-specific. A tissue-specific antigen is expressed in a limited number of tissues. Tissue-specific antigens include, for example, prostate-specific antigen PSA.

Disease-associated antigens can be, for example, tumor antigens, viral antigens, bacterial antigens, fungal antigens, or parasite antigens.

The term “tumor antigen” refers to antigens present expressed exclusively on, associated with, or over-expressed in tumor tissue. Exemplary tumor antigens include, but are not limited to, 5-α-reductase, α-fetoprotein (“AFP”), AM-1, APC, April, B melanoma antigen gene (“BAGE”), β-catenin, Bcl12, bcr-abl, Brachyury, CA-125, caspase-8 (“CASP-8”, also known as “FLICE”), Cathepsins, CD19, CD20, CD21/complement receptor 2 (“CR2”), CD22/BL-CAM, CD23/FcERII, CD33, CD35/complement receptor 1 (“CR1”), CD44/PGP-1, CD45/leucocyte common antigen (“LCA”), CD46/membrane cofactor protein (“MCP”), CD52/CAMPATH-1, CD55/decay accelerating factor (“DAF”), CD59/protectin, CDC27, CDK4, carcinoembryonic antigen (“CEA”), c-myc, cyclooxygenase-2 (“cox-2”), deleted in colorectal cancer gene (“DCC”), DcR3, E6/E7, CGFR, EMBP, Dna78, farnesyl transferase, fibroblast growth factor-8a (“FGF8a”), fibroblast growth factor-8b (“FGF8b”), FLK-1/KDR, folic acid receptor, G250, G melanoma antigen gene family (“GAGE-family”), gastrin 17, gastrin-releasing hormone, ganglioside 2 (“GD2”)/ganglioside 3 (“GD3”)/ganglioside-monosialic acid-2 (“GM2”), gonadotropin releasing hormone (“GnRH”), UDP-GlcNAc:R₁Man(α1-6)R₂ [GlcNAc to Man(α1-6)] β1,6-N-acetylglucosaminyltransferase V (“GnT V”), GP1, gp100/Pme117, gp-100-in4, gp15, gp75/tyrosine-related protein-1 (“gp75/TRP-1”), human chorionic gonadotropin (“hCG”), heparanase, Her2/neu, human mammary tumor virus (“HMTV”), 70 kiloDalton heat-shock protein (“HSP70”), human telomerase reverse transcriptase (“hTERT”), insulin-like growth factor receptor-1 (“IGFR-1”), interleukin-13 receptor (“IL-13R”), inducible nitric oxide synthase (“iNOS”), Ki67, KIAA0205, K-ras, H-ras, N-ras, KSA, LKLR-FUT, melanoma antigen-encoding family (“MAGE-family”, including at least MAGE-1, MAGE-2, MAGE-3, and MAGE-4), mammaglobin, MAP17, Melan-A/melanoma antigen recognized by T-cells-1 (“MART-1”), mesothelin, MIC A/B, MT-MMPs, mucin, testes-specific antigen NY-ESO-1, osteonectin, p15, P170/MDR1, p53, p97/melanotransferrin, PAI-1, platelet-derived growth factor (“PDGF”), μPA, PRAME, probasin, progenipoietin, prostate-specific antigen (“PSA”), prostate-specific membrane antigen (“PSMA”), prostatic acid phosphatase (“PAP”), RAGE-1, Rb, RCAS1, SART-1, SSX-family, STAT3, STn, TAG-72, transforming growth factor-alpha (“TGF-α”), transforming growth factor-beta (“TGF-β”), Thymosin-beta-15, tumor necrosis factor-alpha (“TNF-α”), TP1, TRP-2, tyrosinase, vascular endothelial growth factor (“VEGF”), ZAG, p16INK4, and glutathione-S-transferase (“GST”).

The term “viral antigen” refers to antigens derived from any disease-associated pathogenic virus. Exemplary disease-associated viral antigens include, but are not limited to, antigens derived from adenovirus, Arbovirus, Astrovirus, Coronavirus, Coxsackievirus, Crimean-Congo hemorrhagic fever virus, cytomegalovirus (“CMV”), dengue virus, Ebola virus, Epstein-Barr virus (“EBV”), Foot-and-mouth disease virus, Guanarito virus, Hendra virus, herpes simplex virus-type 1 (“HSV-1”), herpes simplex virus-type 2 (“HSV-2”), human herpesvirus-type 6 (“HHV-6”), human herpesvirus-type 8 (“HHV-8”), hepatitis A virus (“HAV”), hepatitis B virus (“HBV”), hepatitis C virus (“HCV”), hepatitis D virus (“HDV”), hepatitis E virus (“HEV”), human immunodeficiency virus (“HIV”), influenza virus, Japanese encephalitis virus, Junin virus, Lassa virus, Machupo virus, Marburg virus, measles virus, human metapneumovirus, Molluscum contagiosum virus, mumps virus, Newcastle disease virus, Nipha virus, Norovirus, Norwalk virus, human papillomavirus (“HPV”), parainfluenza virus, parvovirus, poliovirus, rabies virus, respiratory syncytial virus (“RSV”), rhinovirus, rotavirus, rubella virus, Sabia virus, severe acute respiratory syndrome virus (“SARS”), varicella zoster virus, variola virus, West Nile virus, and yellow fever virus.

The term “bacterial antigen” refers to antigens derived from any disease-associated pathogenic virus. Exemplary bacterial antigens include, but are not limited to, antigens derived from Bacillus anthracis, Bordetella pertussis, Borrelia burgdorferi, Brucella abortus, Brucella canis, Brucella melitensis, Brucella suis, Burkholderia mallei, Burkholderia pseudomallei, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diptheriae, Enterococcus faecalis, Enterococcus faecium, Escherichia coli, enterotoxigenic Escherichia coli, enteropathogenic Escherichia coli, Escherichia coli) 157: H7, Francisella tularensis, Haemophilus influenza, Helicobacter pylori, Legionella pneumophila, Leptospira interrogans, Listeria monocytogenes, Mycobacterium leprae, Mycobacterium tuberculosis, Mycoplasma pneumoniae, Neisseria gonorrhoeae, Neisseria meningitides, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Vibrio cholerae, and Yersinia pestis.

The term “fungal antigen” refers to antigens derived from any disease-associated pathogenic fungus. Exemplary fungal antigens include, but are not limited to, antigens derived from Aspergillus clavatus, Aspergillus flavus, Aspergillus fumigatus, Aspergillus nidulans, Aspergillus niger, Aspergillus terreus, Blastomyces dermatitidis, Candida albicans, Candida dubliniensis, Candida glabrata, Candida parapsilosis, Candida rugosa, Candida tropicalis, Cryptococcus albidus, Cryptococcus gattii, Cryptococcus laurentii, Cryptococcus neoformans, Histoplasma capsulatum, Microsporum canis, Pneumocystis carinii, Pneumocystis jirovecii, Sporothrix schenckii, Stachbotrys chartarum, Tinea barbae, Tinea captitis, Tinea corporis, Tinea cruris, Tinea faciei, Tinea incognito, Tinea nigra, Tinea versicolor, Trichophyton rubrum and Trichophyton tonsurans.

The term “parasite antigen” refers to antigens derived from any disease-associated pathogenic parasite. Exemplary parasite antigens include, but are not limited to, antigens derived from Anisakis spp. Babesia spp., Baylisascaris procyonis, Cryptosporidium spp., Cyclospora cayetanensis, Diphyllobothrium spp., Dracunculus medinensis, Entamoeba histolytica, Giardia duodenalis, Giardia intestinalis, Giardia lamblia, Leishmania sp., Plasmodium falciparum, Schistosoma mansoni, Schistosoma haematobium, Schistosoma japonicum, Taenia spp., Toxoplasma gondii, Trichinella spiralis, and Trypanosoma cruzi.

“Epstein-Barr Virus,” “EBV,” “human herpesvirus 4” and “HHV-4” interchangeably refer to an oncogenic human herpesvirus. EBV is the cause of acute infectious mononucleosis (AIM, also known as glandular fever). It is also associated with particular forms of cancer, such as Hodgkin's lymphoma. Burkitt's lymphoma, nasopharyngeal carcinoma, and conditions associated with human immunodeficiency virus (HIV), such as hairy leukoplakia and central nervous system lymphomas. EBV infects B cells of the immune system and epithelial cells. Once the virus's initial lytic infection is brought under control, EBV latently persists in the individual's B cells for the rest of the individual's life due to a complex life cycle that includes alternate latent find lytic phases.

“Symptom of EBV infection” includes acute infectious mononucleosis (AIM, also known as glandular fever) and/or the presence of EBV-associated cancer. “EBV-associated cancer” refers to cancer that is caused and/or aggravated, at least in part, by infection with EBV, such as Hodgkin's lymphoma, Burkitt's lymphoma, nasopharyngeal carcinoma, cervical cancer, hairy leukoplakia and central nervous system lymphomas.

The terms “antigen”, “immunogen”, “antigenic”, “immunogenic”, “antigenically active,” and “immunologically active” when made in reference to a molecule, refer to any substance that is capable of inducing a specific humoral and/or cell-mediated immune response. In a particular embodiment, the antigen comprises at least a portion or an ectodomain.

“EBV antigen” refers to an antigen from EBV, such as “gB, gH, gL, and gp350/220” and tumor-associated EBV antigens.

The term “EBV envelope glycoproteins” include gp350, gB, gp42, gH, gL, gM, gN, BMRF2, BDLF2, BDLF3, BILF1, BILF2, and BARF1. The term “T cell antigens” refers to EBNA1, EBNA2, EBNA3a, EBNA3b, EBNA3c, EBNA-leader protein, and LMP2.

The term “gp350/220” is the predominant EBV envelope protein. Interactions between EBVgp350/220 and complement receptor type 2 (CR2) CD21 and/or (CR1)CD35 on B-cells is required for cellular attachment and initiation of latent infection (SEQ ID NO:1)

The term “gH” refers to glycoprotein gp85 precursor of human herpesvirus 4 and is exemplified by SEQ ID NO:2, NCBI Reference Sequence: YP_401700.1.

The term “gL” and “BKRF2” are interchangeably used, and exemplified in SEQ ID NO: 3, NCBI Reference Sequence: YP_001129472.1.

The term “BZLF1-BRLF1fusion” refers to transcriptional activators of the EBV early genes and exemplified in SEQ ID NO: 4.

“EBNA-3A” is exemplified in SEQ ID NO: 5, NCBI Reference Sequence: YP_401677.1.

“Tumor-associated EBV antigens” are EBV antigens that are associated with tumors in subjects who are infected with EBV. Exemplary tumor-associated EBV antigens include EBNA1, LMP1, LMP2, and BARF1, those described in Lin et al. “CD4 and CD8 T cell responses to tumor-associated Epstein-Barr virus antigens in nasopharyngeal carcinoma patients.” Cancer Immunol Immunother. 2008 July; 57 (7): 963-75; Kohrt et al. “Dynamic CD8 T cell responses to tumor-associated Epstein-Barr virus antigens in patients with Epstein-Barr virus-negative Hodgkin's disease,” Oncol Res. 2009; 18 (5-6): 287-92; Parmita et al., “Humoral immune responses to Epstein-Barr virus encoded tumor associated proteins and their putative extracellular domains in nasopharyngeal carcinoma patients and regional controls,” J Med Virol. 2011 April; 83 (4): 665-78.

An “adjuvant” means a vehicle to enhance antigenicity. An adjuvant can include: (1) suspensions of minerals (alum, aluminum hydroxide, and/or phosphate) on which antigen is adsorbed; (2) water-in-oil emulsions in which an antigen solution is emulsified in mineral oil (Freund's incomplete adjuvant), sometimes with the inclusion of killed mycobacteria (Freund's complete adjuvant) to further enhance antigenicity by inhibiting degradation of antigen and/or causing an influx of macrophages; (3) immunostimulatory substances including but not limited oligonucleotides such as, for example, those including a CpG motif can also be used as adjuvants (for example see U.S. Pat. Nos. 6,194,388; and 6,207,646); and (4) purified or recombinant proteins such as costimulatory molecules (e.g., B7-1, ICAM-1, LFA-3, and GM-CSF).

As used herein, “affecting an immune response” includes the development, in a subject, of a humoral and/or a cellular immune response to a protein and/or polypeptide produced by the recombinant MVA or VRP and/or compositions and/or vaccines comprising the recombinant MVA and VRP of the invention. A “humoral” immune response, as this term is well known in the art, refers to an immune response comprising antibodies, while the “cellular” immune response, as this term is well known in the art, refers to an immune response comprising T-lymphocytes and other white blood cells, especially the immunogen-specific response by HLA-restricted cytolytic T-cells, i.e., “CTLs.” A cellular immune response occurs when the processed immunogens, i.e., peptide fragments, are displayed in conjunction with the major histocompatibility complex.

As used herein, the term “alphavirus” has its conventional meaning in the art, and includes the various species of Venezuelan equine encephalitis virus (VEEV), western equine encephalitis virus (WEEV), and eastern equine encephalitis virus (EEEV). “Equine encephalitis virus (EEV)” as used herein includes VEEV, WEEV and EEEV and its strains and isolates.

As used herein, the terms “expressed”, “express”, “expression” and the like which can be used interchangeable denote the transcription alone as well as both the transcription and translation of a sequence of interest. Thus, in referring to expression of a nucleotide sequence present in the form of DNA, the product resulting from this expression may be either RNA (resulting from transcription alone of the sequence to be expressed) or a polypeptide sequence (resulting from both transcription and translation of the sequence to be expressed). The term “expression” thus also includes the possibility that both RNA and polypeptide product result from said expression and remain together in the same shared milieu. For example, this is the case when the mRNA persists following its translation into polypeptide product.

As used herein, the term “expression cassette” is defined as a part of a vector or recombinant virus typically used for cloning and/or transformation. An expression cassette is typically comprised of a) one or more coding sequences (e.g., open reading frame (ORF), genes, nucleic acids encoding a protein and/or antigen), and b) sequences controlling expression of one or more coding sequences (e.g., a promoter). Additionally, an expression cassette may comprise a 3′ untranslated region (e.g., a transcriptional terminator such as a vaccinia transcriptional terminator). “Expression cassette” can be used interchangeable with the term “transcriptional unit”.

“Formulation” refers to a composition containing an active pharmaceutical or biological ingredient e.g., a recombinant MVA of the present invention, along with one or more additional components. The term “formulation” is used interchangeably with the terms “pharmaceutical composition,” “vaccine composition,” and “vaccine formulation” herein. The formulations can be liquid or solid (e.g., lyophilized).

The term “gene” is used broadly to refer to any segment of polynucleotide associated with a biological function. Thus, genes include introns and exons as in genomic sequence, or just the coding sequences as in cDNAs or viral RNA and/or the regulatory sequences required for their expression. For example, gene also refers to a nucleic acid fragment that expresses mRNA or functional RNA, or encodes a specific protein, and which includes regulatory sequences.

As used herein, a “heterologous” gene, nucleic acid, antigen, or protein is understood to be a nucleic acid or amino acid sequence which is not present in the wild-type poxviral genome (e.g., MVA or MVA-BN). The skilled person understands that a “heterologous gene”, when present in a poxvirus such as MVA or MVA-BN, is to be incorporated into the poxviral genome in such a way that, following administration of the recombinant poxvirus to a host cell, it is expressed as the corresponding heterologous gene product, i.e., as the “heterologous antigen” and/or “heterologous protein.” Expression is normally achieved by operatively linking the heterologous gene to regulatory elements that allow expression in the poxvirus-infected cell. Preferably, the regulatory elements include a natural or synthetic poxvirus promoter.

The term “immunogenic composition” or “immunological composition” covers a composition that elicits an immune response against an antigen of interest expressed from the MVA. The term “vaccine or vaccine composition” covers any composition that induces a protective immune response against the antigens of interest, or which efficaciously protects against the antigen of interest, e.g. after administration or injection into the animal or human elicits a protective immune response against the antigen or provides efficacious protection against the antigen expressed from the MVA vector. The composition can be administered alone or can be administered sequentially with other compositions or therapeutic compositions thereby providing a combination composition, a cocktail or multivalent mixture of two or more preferably three, four, five or six compositions.

The term “nucleic acid”, “nucleotide sequence”, “nucleic acid sequence” and “polynucleotide” can be used interchangeable and refers to RNA or DNA that is linear or branched, single or double stranded, or a hybrid thereof. The term also encompasses RNA/DNA hybrids. The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A polynucleotide may comprise modified nucleotides, such as methylated nucleotides and nucleotide analogs, uracyl, other sugars and linking groups such as fluororibose and thiolate, and nucleotide branches. The sequence of nucleotides may be further modified after polymerization, such as by conjugation, with a labeling component. Other types of modifications included in this definition are caps, substitution of one or more of the naturally occurring nucleotides with an analog, and introduction of means for attaching the polynucleotide to proteins, metal ions, labeling components, other polynucleotides or solid support. The polynucleotides can be obtained by chemical synthesis or derived from a microorganism.

The term “open reading frame” (ORF) refers to a sequence of nucleotides, that can be translated into amino acids. Typically, such an ORF contains a start codon, a subsequent region usually having a length which is a multiple of 3 nucleotides, but does not contain a stop codon (TAG, TAA, TGA, UAG, UAA, or UGA) in the given reading frame. Typically, ORFs occur naturally or are constructed artificially, i.e., by gene-technological means. An ORF codes for a protein where the amino acids into which it can be translated form a peptide-linked chain. As used herein, the term “essential ORF” means an ORF which when being experimentally partially or fully deleted e.g., in MVA, the MVA virus replication, growth or both replication and growth are reduced (e.g., by at least 15 fold in the mutant compared to the MVA without deletion). Methods to determine MVA virus replication and growth of the virus are well known to the skilled person. For example methods are described in Vaccinia Virus and Poxvirology, Methods and Protocols, Volume 269 Ed. By Stuart N. Isaacs (Humana Press (2004), see e.g., Chapter 8, Growing Poxviruses and determining Virus Titer, Kotwal and Abrahams). Viral growth rates of MVA may be determined by GFP fluorescence as for example described in Orubu et al. (2012) PLOS One 7: e40167 using e.g., CEF cells or the method as described in Hornemann et al. (2003), Journal of Virology 77:8394-8407.

As used herein, “operably linked” means that the components described are in relationship permitting them to function in their intended manner e.g., a promoter to transcribe the nucleic acid to be expressed. A first nucleic acid sequence is operably linked to a second nucleic acid sequence when the first nucleic acid sequence is placed in a functional relationship with the second nucleic acid sequence. For example, a promoter is operably linked to a coding sequence if the promoter is placed in a position where it can direct transcription of the coding sequence. Generally, operably linked DNA sequences are contiguous and, where necessary to join two protein-coding regions, in the same reading frame.

“Percent (%) sequence homology or identity” with respect to nucleic acid sequences described herein is defined as the percentage of nucleotides in a candidate sequence that are identical with the nucleotides in the reference sequence (i.e., the nucleic acid sequence from which it is derived), after aligning the sequences and introducing gaps, if necessary, to achieve the maximum percent sequence identity, and not considering any conservative substitutions as part of the sequence identity. Alignment for purposes of determining percent nucleotide sequence identity or homology can be achieved in various ways that are within the skill in the art, for example, using publically available computer software such as BLAST, ALIGN, or Megalign (DNASTAR) software. Those skilled in the art can determine appropriate parameters for measuring alignment, including any algorithms needed to achieve maximum alignment over the full length of the sequences being compared.

For example, an appropriate alignment for nucleic acid sequences is provided by the local homology algorithm of Smith and Waterman, (1981), Advances in Applied Mathematics 2:482-489. This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov (1986), Nucl. Acids Res. 14 (6): 6745-6763. An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” The same applies to “percent (%) amino acid identity”, mutatis mutandis. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://http://blast.ncbi.nlm.nih.gov/.

The terms “pharmaceutical”, “pharmaceutical composition” and “medicament” are used interchangeably herein referring to a substance and/or a combination of substances being used for the prevention or treatment of a disease.

“Pharmaceutically acceptable” means that the carrier or excipient, at the dosages and concentrations employed, will not cause any unwanted or harmful effect(s) in the subject(s) to which they are administered.

“Pharmaceutically acceptable carriers” are for example described in Remington's Pharmaceutical Sciences, by E. W. Martin, Mack Publishing Co., Easton, PA, 15th Edition (1975); Remington's Pharmaceutical Sciences, 18th edition, A. R. Gennaro, Ed., Mack Publishing Company (1990); Pharmaceutical Formulation Development of Peptides and Proteins, S. Frokjaer and L. Hovgaard, Eds., Taylor & Francis [2000]; and Handbook of Pharmaceutical Excipients, 3rd edition, A. Kibbe, Ed., Pharmaceutical Press (2000). They describe compositions and formulations using conventional pharmaceutically acceptable carriers suitable for administration of the vectors and compositions disclosed herein. Generally, the nature of the carrier used depends on the particular mode of administration being employed. For example, parenteral formulations usually comprise injectable fluids that include pharmaceutically and physiologically acceptable fluids such as water, physiological saline, balanced salt solutions, aqueous dextrose, glycerol or the like, as a vehicle. For solid compositions (such as powders, pills, tablets, or capsules), conventional non-toxic solid carriers include, for example, pharmaceutical grades of mannitol, lactose, starch, or magnesium stearate. Pharmaceutical compositions can also contain minor amounts of non-toxic auxiliary substances such as wetting or emulsifying agents, preservatives, pH-buffering agents and the like such as, for example, sodium acetate or sorbitan monolaurate.

As used herein, “prevent”, “preventing”, “prevention”, or “prophylaxis” of a disease or infection means preventing that such disease occurs in subject (e.g., human or animal).

The term “prime-boost vaccination” refers to a vaccination strategy using a first, priming injection of a vaccine targeting a specific antigen followed at intervals by one or more boosting injections of the same vaccine. Prime-boost vaccination may be homologous or heterologous. A homologous prime-boost vaccination uses a vaccine comprising the same immunogen and vector for both the priming injection and the one or more boosting injections. A heterologous prime-boost vaccination uses a vaccine comprising the same immunogen for both the priming injection and the one or more boosting injections but different vectors for the priming injection and the one or more boosting injections. For example, a homologous prime-boost vaccination may use a recombinant MVA vector comprising the same nucleic acids expressing alphavirus antigens for both the priming injection and the one or more boosting injections. In contrast, a heterologous prime-boost vaccination may use a recombinant MVA vector comprising nucleic acids expressing one alphavirus protein for the priming injection and another recombinant MVA vector expressing a second one alphavirus protein not contained in the priming injection or vice versa. Heterologous prime-boost vaccination also encompasses various combinations such as, for example, use of a plasmid encoding an immunogen in the priming injection and use of a recombinant MVA encoding the same immunogen in the one or more boosting injections, or use of a recombinant protein immunogen in the priming injection and use of a recombinant MVA vector encoding the same protein immunogen in the one or more boosting injections.

As used herein, the term “promoter” denotes a regulatory region of nucleic acid, usually DNA, located upstream of the sequence of a nucleic acid to be expressed, which contains specific DNA sequence elements, that are recognized and bound e.g., by protein transcription factors and polymerases responsible for synthesizing the RNA from the coding region of the gene being promoted. As promoters are typically immediately adjacent to the gene in question, positions in the promoter are designated relative to the transcriptional start site, where transcription of DNA begins for a particular gene (i.e., positions upstream are negative numbers counting back from −1, for example −100 is a position 100 base pairs upstream). Thus, the promoter sequence may comprise nucleotides until position −1. However, nucleotides from position +1 are not part of the promoter, i.e., in this regard it has to be noted that the translation initiation codon (ATG or AUG) is not part of the promoter. Thus, SEQ ID NOs: 7 or 8 are polynucleotides comprising promoters of the invention. A “natural poxvirus promoter” as used herein means an endogenous promoter of the poxvirus genome. A “synthetic poxvirus promoter” means a recombinant engineered promoter active to direct transcription of the nucleic acid to be expressed by a poxvirus (e.g., MVA in CEF cells). The term “26S promoter” is well known to the skilled person and refers to a subgenomic promoter of a 26S RNA of an alphavirus which is usually contained in a single open reading frame (e.g., of capsid-E3-E2-6K-E1 of VEEV). The mRNA encoding the structural proteins of EEVs e.g., VEEV is usually transcribed from a replication intermediate and a 26S subgenomic RNA promoter.

The terms “protein”, “peptide”, “polypeptide” and “polypeptide fragment” are used interchangeably herein to refer to polymers of amino acid residues of any length. The polymer can be linear or branched, it may comprise modified amino acids or amino acid analogs, and it may be interrupted by chemical moieties other than amino acids. The terms also encompass an amino acid polymer that has been modified naturally or by intervention; for example disulfide bond formation, glycosylation, lipidation, acetylation, phosphorylation, or any other manipulation or modification, such as conjugation with a labeling or bioactive component.

The term “recombinant” when applied to a nucleic acid, vector, e.g., MVA and the like refers to a nucleic acid, vector, or made by an artificial combination of two or more otherwise heterologous segments of nucleic acid sequence, or to a nucleic acid, vector or comprising such an artificial combination of two or more otherwise heterologous segments of nucleic acid sequence. The artificial combination is most commonly accomplished by artificial manipulation of isolated segments of nucleic acids, using well-established genetic engineering techniques. Generally, a “recombinant” MVA as described herein refers to MVAs that are produced by standard genetic engineering methods, i.e., MVAs of the present invention are thus genetically engineered or genetically modified MVAs. The term “recombinant MVA” thus includes MVAs (e.g., MVA-BN) which have stably integrated recombinant nucleic acid, preferably in the form of a transcriptional unit, in their genome. A transcriptional unit may include a promoter, enhancer, terminator and/or silencer. Recombinant MVAs of the present invention may express heterologous antigenic determinants, polypeptides or proteins (antigens) upon induction of the regulatory elements.

As used herein, the term “protective immunity” or “protective immune response” means that the vaccinated subject is able to control an infection with the pathogenic agent against which the vaccination was done. Usually, the subject having developed a “protective immune response” develops only mild to moderate clinical symptoms or no symptoms at all. In cases where the infection would be expected lethal without countermeasures, a subject having a “protective immune response” or “protective immunity” against a certain agent will not die as a result of the infection with said agent.

The term “reference sample” as used herein, refers to a sample which is analyzed in a substantially identical manner as the sample of interest and whose information is compared to that of the sample of interest. A reference sample thereby provides a standard allowing for the evaluation of the information obtained from the sample of interest. A reference sample may be identical to the sample of interest except for one component which may be exchanged, missing or added.

The term “structural protein” of an EEV refers to a structural protein/polyprotein encoded by the RNA of an EEV (e.g., any of the WEEVs, VEEVs or EEEVs as described herein). The structural protein is usually produced by the virus as a structural polyprotein of five proteins i.e., C, E3, E2, 6k and E1 and is represented generally in the literature as C-E3-E2-6k-E1. E3 and 6k are also described as membrane translocation/transport signals for the two glycoproteins, E2 and E1. Nucleotide sequences encoding “structural proteins” as used herein means a nucleotide sequence encoding proteins which are required for encapsidation (e.g., packaging) of the viral genome, and include the capsid protein, E1 glycoprotein, and E2 glycoprotein. “Structural polyprotein” of EEV refers to the polyprotein C-E3-E2-6k-E1 of an EEV.

The term “transcription level” or “protein level” related to a specific promoter as used herein refers to the amount of gene/nucleic acid product present in the body or a sample at a certain point of time. The transcription or protein level (e.g., transcription of nucleic acid as mRNA or protein amount translated form the mRNA) can for example be determined, measured or quantified by means of the mRNA or protein expressed from the gene/polynucleotide e.g., as encoded by the recombinant MVA of the present invention. Gene expression can result in production of the protein, by transcription of the gene by RNA polymerase to produce a messenger RNA (mRNA) that contains the same protein-encoding information and translation of the mRNA by ribosomes to produce the protein. The term “transcribed” or “transcription” refers to the process of copying a DNA sequence of the gene by RNA polymerase into the mRNA, using the DNA as a template. The term “translated” or “translation” refers to the process by which the information contained in the mRNA is used as a blueprint to synthesize the protein. The transcription or protein level can for example be quantified by normalizing the amount mRNA or of protein of interest present in a sample with the total amount of gene product of the same category (mRNA or total protein) in the same sample or a reference sample (e.g., taken at the same time from the same sample). The transcription can be measured or detected by means of any method as known in the art, e.g., methods for the indirect detection and measurement of the gene product of interest that usually work via binding of the gene product of interest with one or more different molecules or detection means (e.g., primer(s), probes, antibodies, protein scaffolds) specific for the gene product of interest. Such methods include for example RT-PCR and/or quantitative PCR. The determination of the level of protein can be measured or detected by means of any known method as known to the artisan, e.g., western blot, ELISA, or mass spectrometry.

As used herein, “transcriptional terminator” is comprised of a DNA sequences involved in specific termination of an RNA transcript by an RNA polymerase. Vaccinia virus including MVA RNA polymerase terminates transcription downstream of an RNA signal (UUUUUNU, TTTTTNT or T5NT on the DNA level) in the nascent RNA (Earl et al. (1990), J. Virol. 64:2448-2451). “Transcriptional terminator” is sometimes referred to as a “termination signal” in the literature and thus can be used interchangeable.

As used herein, “treat”, “treating” or “treatment” of a disease means the prevention, reduction, amelioration, partial or complete alleviation, or cure of a disease e.g., an EEV-caused disease. It can be one or more of reducing the severity of the disease, limiting or preventing development of symptoms characteristic of the disease being treated, inhibiting worsening of symptoms characteristic of the disease being treated, limiting or preventing recurrence of the disease in a subject who has previously had the disease, and limiting or preventing recurrence of symptoms in subjects.

As used herein, “trivalent” in combination with vaccine or recombinant MVA means that the vaccine or recombinant MVA has a valence against three different viruses and generates a protective immune response against antigens (e.g., structural proteins or structural polyproteins) of those different viruses. Thus, in the context of a trivalent MVA vaccine of the invention trivalent means a valence against three different viruses of which antigens are encoded by the MVA vaccine or vaccine comprising a recombinant MVA expressing the nucleic acids encoding for the antigens e.g., structural proteins or structural polyproteins of VEEV, WEEV and EEEV. Another example for trivalent which is also covered by the meaning of trivalent is that the three different viruses are different virus strains e.g., two WEEV strains such as for example 71V-1658 and Fleming in addition to a VEEV or EEEV strain. In the latter case the recombinant MVA of the present invention for example comprises a nucleotide sequence encoding for the proteins (e.g., structural protein, structural polyprotein, envelope protein) of WEEV 71V-1658, WEEV Fleming and of an EEEV strain e.g., EEEV V105-00210. In comparison “monovalent” means that the vaccine or recombinant MVA has a valence against only one virus of a particular species, such as only VEEV, only WEEV or only EEEV and generates a protective immune response against only one structural protein or structural polyprotein of one virus. It does not exclude however the generation of protective immune responses against several closely related virus subtypes. “Divalent” thus means that the vaccine or recombinant MVA has a valence against two viruses.

A “vector” refers to a recombinant DNA or RNA plasmid or virus that comprises a heterologous polynucleotide to be delivered to a target cell, either in vitro or in vivo. The heterologous polynucleotide may comprise a sequence of interest for purposes of prevention or therapy and may optionally be in the form of an expression cassette. As used herein, a vector needs not be capable of replication in the ultimate target cell or subject. The term includes cloning vectors and viral vectors.

The term “viral replicon” as used in the context of the present invention is used to refer to RNA or DNA comprising portions of the 49S viral genomic RNA that are essential for transcription and for cytoplasmic amplification of the transported RNA and for subgenomic RNA expression of a heterologous nucleic acid sequence. Thus, the replicon encodes and expresses viral non-structural proteins necessary for cytoplasmic amplification of the virus RNA.

In the context of the present invention the term “virus” or “recombinant virus” refers to an infectious or non-infectious virus comprising a viral genome. In this case the nucleic acids, promoters, recombinant proteins, and/or expression cassettes as mentioned herein are part of the viral genome of the respective recombinant virus. The recombinant viral genome is packaged and the obtained recombinant viruses can be used for the infection of cells and cell lines, in particular for the infection of living animals including humans.

The term “TCID₅₀” is the abbreviation of “tissue culture infectious dose”, that amount of a pathogenic agent that will produce pathological change in 50% of cell cultures inoculated, expressed as TCID₅₀/ml. A method for determining TCID₅₀ is well known to the person skilled in the art. It is for example described in e.g., Example 2 of WO 03/053463.

The term “subject” as used herein is a living multi-cellular vertebrate organisms, including, for example, humans, non-human mammals and (non-human) primates. The term “subject” may be used interchangeably with the term “animal” herein.

### Self-Amplifying RNA

There are currently two different types of synthetic RNA vaccines: Conventional mRNA and self-amplifying RNA (saRNA). Use of conventional mRNA strategies (also referred to as nonreplicating or non-amplifying mRNA) against infectious diseases and cancers has been investigated in several preclinical and clinical trials. In vitro transcribed mRNAs encoding viral antigens have been explored as vaccines, while those encoding therapeutic proteins, such as antibodies or immune modulators, have been considered for immunotherapy. The incorporation of chemically modified nucleotides, sequence optimization, and different purification strategies improve efficiency of mRNA translation and reduce intrinsic immunogenic properties. However, antigen expression is proportional to the number of conventional mRNA transcripts successfully delivered during vaccination. Achieving adequate expression for protection or immunomodulation may thus require large doses or repeat administrations. saRNA vaccines, which are genetically engineered replicons derived from self-replicating single-stranded RNA viruses address this limitation. They can be delivered as viral replicon particles (VRPs) with the saRNA packaged into the viral particle, or as a completely synthetic saRNA produced after in vitro transcription. To generate replication-defective VRPs, envelope proteins are provided in trans as defective helper constructs during production. Resulting VRPs therefore lack the ability to form infectious viral particles following a first infection, and only the RNA is capable of further amplification. VRPs may be derived from both positive-sense and negative-sense RNA viruses, however the latter are more complex and require reverse genetics to rescue the VRPs. As with gene therapy, there are several issues associated with the use of viral vectors for vaccine development. These include immunogenicity of the vector itself, which can elicit an undesirable immune response and prevent subsequent booster administrations using the same vector. Preexisting immunity to the viral vector can also render a vaccine ineffective. As with live-attenuated vaccines, replication-competent alphavirus vectors also pose the threat of viral reactivation. To circumvent this, saRNA vaccines can be produced and delivered in a similar manner to conventional mRNA vaccines. Positive-sense alphavirus genomes that have been commonly used for saRNA vaccine design include the Venezuelan equine encephalitis virus (VEE), Sindbis virus (SINV), and Semliki forest virus (SFV). The alphavirus replicase genes encode an RNA-dependent RNA polymerase (RdRP) complex which amplifies synthetic transcripts in situ. The antigenic or therapeutic sequence is expressed at high levels as a separate entity and further proteolytic processing of the immunogen is not required. As a result of their self-replicative activity, saRNAs can be delivered at lower concentrations than conventional mRNA vaccines to achieve comparable antigen expression.

As mentioned above, the saRNA constructs have historically been delivered from alphaviruses, such as the Venezuelan equine encephalitis virus (VEEV), Semliki Forest virus (SFV) or Sindbis virus. These saRNA constructs contain the four non-structural proteins, a subgenomic promoter, and the gene of interest (replacing the viral structural proteins). By deleting the viral structural proteins, the RNA is incapable of producing an infectious virus. After delivery to the cytoplasm, the non-structural proteins form an RNA-dependent RNA polymerase (RDRP) that replicates both the genomic RNA (entire RNA strand) and subgenomic RNA (gene of interest). Each of the four non-structural proteins plays a role in the formation of the RDRP, which is a complex and multistage process. This RNA replication is what leads to higher antigen expression than non-replicating mRNA.

### EEV Viruses, Proteins and Nucleotide Sequences

EEV are alphavirus belonging to the family of Togaviridae. EEV are small, enveloped positive-strand RNA viruses well known in the art. The viral nucleocapsid is surrounded by host derived lipid membranes in which a trimer of envelope proteins of E1 and E2 heterodimers are embedded. The nucleocapsid consists of a capsid protein (C) surrounding the single-strand RNA genome. The RNA genome (49S RNA) of EEV viruses is approximately 11-12 kb in length and contains a 5′ cap and 3′ polyadenylation tail and is immediately translated upon entry into the cell. The 5′ region of the genome encodes for four non-structural proteins (NSP1, NSP2, NSP3, and NSP4). The 3′ region of the genome encodes for five structural proteins (C, E3, E2, 6k, E1) which are expressed as a structural polyprotein from 26S subgenomic RNA. The mRNA encoding for the structural proteins is transcribed from a replication intermediate and a 26S subgenomic promoter. Protease cleavage of the polyprotein produces the mature structural proteins C, E3, E2, 6k, E1.

The nucleocapsid (C) protein possesses auto-proteolytic activity which cleaves the C protein from the precursor protein soon after the ribosome transits the junction between the C and E3 protein coding sequence. Subsequently, the envelope glycoproteins E2 and E1 are derived by proteolytic cleavage and form heterodimers. E2 initially appears in the infected cell as a precursor, pE2, which consists of E3 and E2. After glycosylation and transit through the endoplasmic reticulum and the Golgi apparatus, E3 is cleaved from E2 by furin-like protease activity at a cleavage site.

Live-attenuated vaccines have been used in the US military and laboratory workers and formalin-inactivated vaccines are available for use in horses.

One such live-attenuated vaccine is TC-83, originally developed by the US Army for vaccine use (Pittman et al., 1996). TC-83 was created by serially passaging the Trinidad Donkey VEEV strain in guinea pig heart cells (Alevizatos et al., 1967). Point mutations in E2 and the 5′ untranslated region are responsible for the attenuated phenotype of TC-83 (Kinney et al., 1993). TC-83 has been noted to be effective in preventing disease in humans, but 15-37.5% of vaccine recipients develop febrile symptoms (Berge et al., 1961; Mckinney et al., 1963; Alevizatos et al., 1967; Pittman et al., 1996) and only 82% of vaccinees seroconvert upon vaccination. The probability of plaque reduction neutralization titer remaining ≥1:20 over a period of 5-8 years was 60%. Since TC-83 is only available for use as an investigational vaccine and the population to which it is available is limited, additional studies to evaluate the immunogenicity of the vaccine in humans over time are not available. Of interest to this study is that following intranasal infection with the vaccine strain of VEEV-TC-83, C57BL/6 (WT) mice develop disseminated infection of the brain with high infectious titers without mortality (Hart et al., 1997; Steele et al., 1998; Julander et al., 2008; Taylor et al., 2012).

It is to be understood that also any combination of any WEEV, EEEV and/or VEEV as mentioned above is also encompassed with any of the embodiments as described herein.

### Modified Vaccinia Virus Ankara (MVA)

The man-made attenuated modified vaccinia virus Ankara (“MVA”) was generated by more than 570 serial passages on chicken embryo fibroblasts of the chorioallantois vaccinia virus Ankara (CVA) strain (for review see Mayr, A., et al. Infection 3, 6-14 (1975)). As a consequence of these long-term passages, the genome of the resulting MVA virus had about 27 kilobases of genomic sequence deleted as compared to its predecessor CVA and, therefore, was described as highly host cell restricted for replication to avian cells (Meyer, H. et al., J. Gen. Virol. 72, 1031-1038 (1991), Meisinger et al. J. Gen. Virol 88, 3249-3259 (2007)). It was shown in a variety of animal models that the resulting MVA was avirulent compared to the fully replication competent starting material (Mayr, A. & Danner, K., Dev. Biol. Stand. 41:225-34 (1978)).

An MVA virus useful in the practice of the present invention can include, but is not limited to, MVA-572 (deposited as ECACC V94012707 on Jan. 27, 1994); MVA-575 (deposited as ECACC V00120707 on Dec. 7, 2000), MVA-1721 (referenced in Suter et al., Vaccine 2009), NIH clone 1 (deposited as ATCCR PTA-5095 on Mar. 27, 2003) and MVA-BN (deposited at the European Collection of Cell Cultures (ECACC) under number V00083008 on Aug. 30, 2000).

More preferably the MVA used in accordance with the present invention includes MVA-BN and MVA-BN derivatives. MVA-BN has been described in International PCT publication WO 02/042480. “MVA-BN derivatives” refer to any virus exhibiting essentially the same replication characteristics as MVA-BN, as described herein, but exhibiting differences in one or more parts of their genomes.

MVA-BN, as well as MVA-BN derivatives, is replication incompetent, meaning a failure to reproductively replicate in vivo and in vitro. More specifically in vitro, MVA-BN or MVA-BN derivatives have been described as being capable of reproductive replication in chicken embryo fibroblasts (CEF), but not capable of reproductive replication in the human keratinocyte cell line HaCat (Boukamp et al (1988), J. Cell Biol. 106:761-771), the human bone osteosarcoma cell line 143B (ECACC Deposit No. 91112502), the human embryo kidney cell line 293 (ECACC Deposit No. 85120602), and the human cervix adenocarcinoma cell line HeLa (ATCC Deposit No. CCL-2). Additionally, MVA-BN or MVA-BN derivatives have a virus amplification ratio at least two fold less, more preferably three-fold less than MVA-575 in Hela cells and HaCaT cell lines. Tests and assay for these properties of MVA-BN and MVA-BN derivatives are described in WO 02/42480 (U.S. Patent application No. 2003/0206926) and WO 03/048184 (U.S. Patent application No. 2006/0159699).

The term “not capable of reproductive replication” or “no capability of reproductive replication” in human cell lines in vitro as described in the previous paragraphs is, for example, described in WO 02/42480, which also teaches how to obtain MVA having the desired properties as mentioned above. The term applies to a virus that has a virus amplification ratio in vitro at 4 days after infection of less than 1 using the assays described in WO 02/42480 or in U.S. Pat. No. 6,761,893.

The amplification or replication of a virus in human cell lines in vitro as described in the previous paragraphs is normally expressed as the ratio of virus produced from an infected cell (output) to the amount originally used to infect the cell in the first place (input) referred to as the “amplification ratio”. An amplification ratio of “1” defines an amplification status where the amount of virus produced from the infected cells is the same as the amount initially used to infect the cells, meaning that the infected cells are permissive for virus infection and reproduction. In contrast, an amplification ratio of less than 1, i.e., a decrease in output compared to the input level, indicates a lack of reproductive replication and therefore attenuation of the virus.

Integration Sites into MVA

Nucleotide sequences encoding for one or more protein(s) may be inserted into any suitable part of the virus or viral vector, in particular the viral genome of the recombinant MVA. Suitable parts of the recombinant MVA are non-essential parts of the MVA genome. Non-essential parts of the MVA genome may be intergenic regions or the known deletion sites 1-6 of the MVA genome. Alternatively or additionally, non-essential parts of the recombinant MVA can be a coding region of the MVA genome which is non-essential for viral growth. However, the insertion sites are not restricted to these preferred insertion sites in the MVA genome, since it is within the scope of the present invention that the promoter, expression cassette and/or nucleotide encoding for one, two three or more protein(s) as described herein may be inserted anywhere in the viral genome as long as it is possible to obtain recombinants that can be amplified and propagated in at least one cell culture system, such as Chicken Embryo Fibroblasts (CEF cells). Preferably, the nucleotide sequences encoding for one, two, three or more protein(s) may be inserted into one or more intergenic regions (IGR) of the MVA. The term “intergenic region” refers preferably to those parts of the viral genome located between two adjacent open reading frames (ORF) of the MVA virus genome, preferably between two essential ORFs of the MVA virus genome. In certain embodiments, the IGR is selected from IGR 07/08, IGR 44/45, IGR 64/65, IGR 88/89, IGR 136/137, and IGR 148/149. In certain embodiments, less than 5, 4, 3 or 2 IGRs of the recombinant MVA comprise nucleotide sequences encoding for one or more protein(s). The number of insertion sites of MVA comprising nucleotide sequences encoding for one or more protein(s) can be 1, 2, 3, 4, 5, 6, 7, or more. In certain embodiments, the nucleotide sequences are inserted into 4, 3, 2, or fewer insertion sites. Preferably, two insertion sites are used, preferably IGR 44/45 and IGR 88/89. In certain embodiments, three insertion sites are used. Preferably, the recombinant MVA comprises at least 2, 3, 4, 5, 6, or 7 genes inserted into 2 or 3 insertion sites.

The nucleotide sequences may, additionally or alternatively, be inserted into one or more of the known deletion sites, i.e., deletion sites I, II, III, IV, V, or VI of the MVA genome. The term “known deletion site” refers to those parts of the MVA genome that were deleted through continuous passaging on CEF cells characterized at passage 516 with respect to the genome of the parental virus from which the MVA is derived from, in particular the parental chorioallantois vaccinia virus Ankara (CVA) e.g., as described in Meisinger-Henschel et al. (2007), Journal of General Virology 88:3249-3259. In certain embodiments, less than 5, 4, 3, or 2 of the known deletion sites of the recombinant MVA comprise nucleotide sequences encoding for one, two, three or more protein(s) as described herein.

The recombinant MVA viruses provided herein can be generated by routine methods known in the art. Methods to obtain recombinant MVAs or to insert exogenous coding sequences into a MVA genome are well known to the person skilled in the art. For example, methods for standard molecular biology techniques such as cloning of DNA, DNA and RNA isolation, Western blot analysis, RT-PCR and PCR amplification techniques are described in Molecular Cloning, A laboratory Manual 2nd Ed. (J. Sambrook et al., Cold Spring Harbor Laboratory Press (1989)), and techniques for the handling and manipulation of viruses are described in Virology Methods Manual (B. W. J. Mahy et al. (eds.), Academic Press (1996)). Similarly, techniques and know-how for the handling, manipulation and genetic engineering of MVA are described in Molecular Virology: A Practical Approach (A. J. Davison & R. M. Elliott (Eds.), The Practical Approach Series, IRL Press at Oxford University Press, Oxford, UK (1993), see, e.g., Chapter 9: Expression of genes by Vaccinia virus vectors) and Current Protocols in Molecular Biology (John Wiley & Son, Inc. (1998), see, e.g., Chapter 16, Section IV: Expression of proteins in mammalian cells using vaccinia viral vector).

For the generation of the various recombinant MVAs disclosed herein, different methods known to the person skilled in the art may be applicable. The DNA sequence to be inserted into the virus can be placed into an E. coli plasmid construct into which DNA homologous to a section of DNA of the MVA has been inserted. Separately, the DNA sequence to be inserted can be ligated to a promoter. The promoter-gene linkage can be positioned in the plasmid construct so that the promoter-gene linkage is flanked on both ends by DNA homologous to a DNA sequence flanking a region of MVA DNA containing a non-essential locus. The resulting plasmid construct can be amplified by propagation within E. coli bacteria and isolated. The isolated plasmid containing the DNA gene sequence to be inserted can be transfected into a cell culture, e.g., of chicken embryo fibroblasts (CEFs), at the same time the culture is infected with MVA. Recombination between homologous MVA DNA in the plasmid and the viral genome, respectively, can generate an MVA modified by the presence of foreign DNA sequences.

According to a preferred embodiment, a cell of a suitable cell culture as, e.g., CEF cells, can be infected with the MVA. The infected cell can be, subsequently, transfected with a first plasmid vector comprising a foreign or heterologous gene or genes, preferably under the transcriptional control of an expression control element. As explained above, the plasmid vector also comprises sequences capable of directing the insertion of the exogenous sequence into a selected part of the MVA genome. Optionally, the plasmid vector also contains a cassette comprising a marker and/or selection gene operably linked to a poxvirus promoter. Suitable marker or selection genes are, e.g., the genes encoding the green fluorescent protein, β-galactosidase, neomycin-phosphoribosyltransferase or other markers. The use of selection or marker cassettes simplifies the identification and isolation of the generated recombinant MVA.

However, a recombinant MVA can also be identified by PCR technology. Subsequently, a further cell can be infected with the recombinant MVA obtained as described above and transfected with a second vector comprising a second foreign or heterologous gene or genes. In case, this gene shall be introduced into a different insertion site of the MVA genome, the second vector also differs in the MVA-homologous sequences directing the integration of the second foreign gene or genes into the genome of the MVA. After homologous recombination has occurred, the recombinant virus comprising two or more foreign or heterologous genes can be isolated. For introducing additional foreign genes into the recombinant virus, the steps of infection and transfection can be repeated by using the recombinant virus isolated in previous steps for infection and by using a further vector comprising a further foreign gene or genes for transfection.

Alternatively, the steps of infection and transfection as described above are interchangeable, i.e., a suitable cell can at first be transfected by the plasmid vector comprising the foreign gene and, then, infected with the MVA. As a further alternative, it is also possible to introduce each foreign gene into different viruses, co-infect a cell with all the obtained recombinant viruses and screen for a recombinant including all foreign genes. A third alternative is ligation of DNA genome and foreign sequences in vitro and reconstitution of the recombined vaccinia virus DNA genome using a helper virus. A fourth alternative is homologous recombination in E. coli or another bacterial species between a vaccinia virus genome cloned as a bacterial artificial chromosome (BAC) and a linear foreign sequence flanked with DNA sequences homologous to sequences flanking the desired site of integration in the vaccinia virus genome.

### Expression of Antigenic Proteins

In certain embodiments, expression of one, more, or all of the nucleotide sequences encoding for a protein (disease-associated antigen) of, e.g. the EBV virus as described herein is under the control of one or more poxvirus promoters. The promoter according to the present invention may be any synthetic or natural poxvirus promoter. In certain embodiments, the poxvirus promoter is a Pr13.5 promoter, a PrHyb promoter, a Pr7.5 promoter, a hybrid early/late promoter, a PrS promoter, a PrS5E promoter, Pr1328, PrH5m, a synthetic or natural early or late promoter, or a cowpox virus ATI promoter. Suitable promoters are further described in WO 2010/060632, WO 2010/102822, WO 2013/189611 and WO 2014/063832.

Nucleic acids encoding the disease-associated antigen can be operatively linked to expression control sequences. An expression control sequence operatively linked to a coding sequence is joined such that expression of the coding sequence is achieved under conditions compatible with the expression control sequences. The expression control sequences include, but are not limited to, appropriate promoters, enhancers, transcription terminators, a start codon at the beginning a protein-encoding open reading frame, splicing signals for introns, and in-frame stop codons. Suitable promoters include, but are not limited to, the SV40 early promoter, the retrovirus LTR, the adenovirus major late promoter, the human CMV immediate early I promoter, and various poxvirus promoters including, but not limited to the following vaccinia virus or MVA-derived promoters: the 30K promoter, the 13 promoter, the sE/L promoter, the Pr7.5K, the 40K promoter, the C1 promoter, the PrSynIlm promoter, the PrLE1 promoter, the PrH5m promoter, the PrS promoter, a hybrid early/late promoter, the PrS5E promoter, the PrA5E promoter, and the Pr4LS5E promoter; a cowpox virus ATI promoter, or the following fowlpox-derived promoters: the Pr7.5K promoter, the 13 promoter, the 30K promoter, or the 40K promoter.

In certain embodiments, the poxvirus promoter is selected from the group consisting of the PrS promoter (SEQ ID NO: 8), Pr1328 (SEQ ID NO: 10), PrH5m (SEQ ID NO: 9.) and the Pr13.5 promoter (SEQ ID NO: 7).

### Antigenic Determinants/Proteins

The term “antigenic determinant” or “antigenic protein” refers to any molecule that stimulates a host's immune system to make an antigen-specific immune response, whether a cellular response or a humoral antibody response. Antigenic determinants may include proteins, polypeptides, antigenic protein fragments, antigens, and epitopes which still elicit an immune response in a host and form part of an antigen, homologues or variants of proteins, polypeptides, and antigenic protein fragments, antigens and epitopes including, for example, glycosylated proteins, polypeptides, antigenic protein fragments, antigens and epitopes, and nucleotide sequences encoding such molecules. Thus, proteins, polypeptides, antigenic protein fragments, antigens and epitopes are not limited to particular native nucleotide or amino acid sequences but encompass sequences identical to the native sequence as well as modifications to the native sequence, such as deletions, additions, insertions and substitutions.

The term “epitope” refers to a site on an antigen to which B- and/or T-cells respond, either alone or in conjunction with another protein such as, for example, a major histocompatibility complex (“MHC”) protein or a T-cell receptor. Epitopes can be formed both from contiguous amino acids or noncontiguous amino acids juxtaposed by secondary and/or tertiary folding of a protein. Epitopes formed from contiguous amino acids are typically retained on exposure to denaturing solvents, while epitopes formed by tertiary folding are typically lost on treatment with denaturing solvents. An epitope typically includes at least 5, 6, 7, 8, 9, 10 or more amino acids—but generally less than 20 amino acids—in a unique spatial conformation. Methods of determining spatial conformation of epitopes include, for example, x-ray crystallography and 2-dimensional nuclear magnetic resonance. See, e.g., “Epitope Mapping Protocols” in Methods in Molecular Biology, Vol. 66, Glenn E. Morris, Ed (1996).

Preferably, a homologue or variant has at least about 50%, at least about 60% or 65%, at least about 70% or 75%, at least about 80%, 81%, 82%, 83%, 84%, 85%, 86%, 87%, 88%, or 89%, more typically, at least about 90%, 91%, 92%, 93%, or 94% and even more typically at least about 95%, 96%, 97%, 98% or 99%, most typically, at least about 99% identity with the referenced protein, polypeptide, antigenic protein fragment, antigen and epitope at the level of nucleotide or amino acid sequence.

In some embodiments, the heterologous nucleic acid encodes antigenic domains or antigenic protein fragments rather than the entire antigenic protein. These fragments can be of any length sufficient to be antigenic or immunogenic. Fragments can be at least 8 amino acids long, preferably 10-20 amino acids, but can be longer, such as, e.g., at least 50, 100, 200, 500, 600, 800, 1000, 1200, 1600, 2000 amino acids long, or any length in between.

In some embodiments, at least one nucleic acid fragment encoding an antigenic protein fragment or immunogenic polypeptide thereof is inserted into the viral vector of the invention. In another embodiment, about 2-6 different nucleic acids encoding different antigenic proteins are inserted into one or more of the viral vectors. In some embodiments, multiple immunogenic fragments or subunits of various proteins can be used. For example, several different epitopes from different sites of a single protein or from different proteins of the same strain, or from a protein orthologue from different strains can be expressed from the vectors.

### Immunogenic Compositions and Disease-Associated Antigens

In one aspect, provided herein are immunogenic compositions comprising recombinant poxviruses such as, for example, modified vaccinia virus Ankara (MVA) comprising a nucleic acid sequence encoding a heterologous disease-associated antigen as well as VRPs encoding the same.

In certain embodiments, the heterologous disease-associated antigen is an infectious disease antigen or a tumor-associated antigen. In certain embodiments, the heterologous disease-associated antigen is a tumor-associated antigen. In certain embodiments, the tumor-associated antigen is selected from the group consisting of 5-α-reductase, α-fetoprotein (“AFP”), AM-1, APC, April, B melanoma antigen gene (“BAGE”), β-catenin, Bcl12, bcr-abl, Brachyury, CA-125, caspase-8 (“CASP-8”, also known as “FLICE”), Cathepsins, CD19, CD20, CD21/complement receptor 2 (“CR2”), CD22/BL-CAM, CD23/FcERII, CD33, CD35/complement receptor 1 (“CR1”), CD44/PGP-1, CD45/leucocyte common antigen (“LCA”), CD46/membrane cofactor protein (“MCP”), CD52/CAMPATH-1, CD55/decay accelerating factor (“DAF”), CD59/protectin, CDC27, CDK4, carcinoembryonic antigen (“CEA”), c-myc, cyclooxygenase-2 (“cox-2”), deleted in colorectal cancer gene (“DCC”), DcR3, E6/E7, CGFR, EMBP, Dna78, farnesyl transferase, fibroblast growth factor-8a (“FGF8a”), fibroblast growth factor-8b (“FGF8b”), FLK-1/KDR, folic acid receptor, G250, G melanoma antigen gene family (“GAGE-family”), gastrin 17, gastrin-releasing hormone, ganglioside 2 (“GD2”)/ganglioside 3 (“GD3”)/ganglioside-monosialic acid-2 (“GM2”), gonadotropin releasing hormone (“GnRH”), UDP-GlcNAc:R₁Man(α1-6)R₂ [GlcNAc to Man(α1-6)] β1,6-N-acetylglucosaminyltransferase V (“GnT V”), GP1, gp100/Pme117, gp-100-in4, gp15, gp75/tyrosine-related protein-1 (“gp75/TRP-1”), human chorionic gonadotropin (“hCG”), heparanase, Her2/neu, human mammary tumor virus (“HMTV”), 70 kiloDalton heat-shock protein (“HSP70”), human telomerase reverse transcriptase (“hTERT”), insulin-like growth factor receptor-1 (“IGFR-1”), interleukin-13 receptor (“IL-13R”), inducible nitric oxide synthase (“iNOS”), Ki67, KIAA0205, K-ras, H-ras, N-ras, KSA, LKLR-FUT, melanoma antigen-encoding family (“MAGE-family”, including at least MAGE-1, MAGE-2, MAGE-3, and MAGE-4), mammaglobin, MAP17, Melan-A/melanoma antigen recognized by T-cells-1 (“MART-1”), mesothelin, MIC A/B, MT-MMPs, mucin, testes-specific antigen NY-ESO-1, osteonectin, p15, P170/MDR1, p53, p97/melanotransferrin, PAI-1, platelet-derived growth factor (“PDGF”), uPA, PRAME, probasin, progenipoietin, prostate-specific antigen (“PSA”), prostate-specific membrane antigen (“PSMA”), prostatic acid phosphatase (“PAP”), RAGE-1, Rb, RCAS1, SART-1, SSX-family, STAT3, STn, TAG-72, transforming growth factor-alpha (“TGF-α”), transforming growth factor-beta (“TGF-β”), Thymosin-beta-15, tumor necrosis factor-alpha (“TNF-α”), TP1, TRP-2, tyrosinase, vascular endothelial growth factor (“VEGF”), ZAG, p16INK4, and glutathione-S-transferase (“GST”). In certain embodiments, the tumor-associated antigen is brachyury. In certain embodiments, the tumor-associated antigen is PSA. In certain embodiments, the tumor-associated antigen is CEA. In certain embodiments, the tumor-associated antigen is MUC-1. In certain embodiments, the tumor-associated antigen is CEA and MUC-1.

In certain embodiments, the heterologous disease-associated antigen is an infectious disease antigen. In certain embodiments, the infectious disease antigen is a viral antigen, a bacterial antigen, a fungal antigen, or a parasite antigen.

In certain embodiments, the infectious disease antigen is a viral antigen. In certain embodiments, the viral antigen is derived from a virus selected from the group consisting of adenovirus, Arbovirus, Astrovirus, Coronavirus, Coxsackievirus, Crimean-Congo hemorrhagic fever virus, cytomegalovirus (“CMV”), dengue virus, Ebola virus, Epstein-Barr virus (“EBV”), Foot-and-mouth disease virus, Guanarito virus, Hendra virus, herpes simplex virus-type 1 (“HSV-1”), herpes simplex virus-type 2 (“HSV-2”), human herpesvirus-type 6 (“HHV-6”), human herpesvirus-type 8 (“HHV-8”), hepatitis A virus (“HAV”), hepatitis B virus (“HBV”), hepatitis C virus (“HCV”), hepatitis D virus (“HDV”), hepatitis E virus (“HEV”), human immunodeficiency virus (“HIV”), influenza virus, Japanese encephalitis virus, Junin virus, Lassa virus, Machupo virus, Marburg virus, measles virus, human metapneumovirus, Molluscum contagiosum virus, mumps virus, Newcastle disease virus, Nipha virus, Norovirus, Norwalk virus, human papillomavirus (“HPV”), parainfluenza virus, parvovirus, poliovirus, rabies virus, respiratory syncytial virus (“RSV”), rhinovirus, rotavirus, rubella virus, Sabia virus, severe acute respiratory syndrome virus (“SARS”), varicella zoster virus, variola virus, West Nile virus, and yellow fever virus.

In certain embodiments, the infectious disease antigen is a bacterial antigen. In certain embodiments, the bacterial antigen is selected from the group consisting of antigens derived from Bacillus anthracis, Bordetella pertussis, Borrelia burgdorferi, Brucella abortus, Brucella canis, Brucella melitensis, Brucella suis, Burkholderia mallei, Burkholderia pseudomallei, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diptheriae, Enterococcus faecalis, Enterococcus faecium, Escherichia coli, enterotoxigenic Escherichia coli, enteropathogenic Escherichia coli, Escherichia coli) 157: H7, Francisella tularensis, Haemophilus influenza, Helicobacter pylori, Legionella pneumophila, Leptospira interrogans, Listeria monocytogenes, Mycobacterium leprae, Mycobacterium tuberculosis, Mycoplasma pneumoniae, Neisseria gonorrhoeae, Neisseria meningitides, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Vibrio cholerae, and Yersinia pestis. In certain embodiments, the bacterial antigen is derived from Bacillus anthracis.

In certain embodiments, the infectious disease antigen is a fungal antigen. In certain embodiments, the fungal antigen is selected from the group consisting of antigens derived from Aspergillus clavatus, Aspergillus flavus, Aspergillus fumigatus, Aspergillus nidulans, Aspergillus niger, Aspergillus terreus, Blastomyces dermatitidis, Candida albicans, Candida dubliniensis, Candida glabrata, Candida parapsilosis, Candida rugosa, Candida tropicalis, Cryptococcus albidus, Cryptococcus gattii, Cryptococcus laurentii, Cryptococcus neoformans, Histoplasma capsulatum, Microsporum canis, Pneumocystis carinii, Pneumocystis jirovecii, Sporothrix schenckii, Stachbotrys chartarum, Tinea barbae, Tinea captitis, Tinea corporis, Tinea cruris, Tinea faciei, Tinea incognito, Tinea nigra, Tinea versicolor, Trichophyton rubrum and Trichophyton tonsurans.

In certain embodiments, the infectious disease antigen is a parasite antigen. In certain embodiments, the parasite antigen is selected from the group consisting of antigens derived from Anisakis spp. Babesia spp., Baylisascaris procyonis, Cryptosporidium spp., Cyclospora cayetanensis, Diphyllobothrium spp., Dracunculus medinensis, Entamoeba histolytica, Giardia duodenalis, Giardia intestinalis, Giardia lamblia, Leishmania sp., Plasmodium falciparum, Schistosoma mansoni, Schistosoma haematobium, Schistosoma japonicum, Taenia spp., Toxoplasma gondii, Trichinella spiralis, and Trypanosoma cruzi.

### Epstein-Barr-Virus (EBV)

Epstein-Barr-virus (EBV; also known as human herpesvirus 4, HHV-4) is a human herpesvirus. The enveloped dsDNA virus is transmitted via the oral route by saliva or genital secretions and can infect epithelial cells and B cells, where it enters a bi-phasic lifecycle consisting of lytic and latency phases that are coordinated by complex regulation. The lytic phase, when occurring in young, previously naïve adolescents, can lead to infectious mononucleosis (glandular fever). Infectious mononucleosis is a risk factor for cancer development at later stages. EBV is associated with particular forms of cancer, such as Hodgkin's lymphoma, Burkitt's lymphoma, gastric cancer and nasopharyngeal carcinoma. Furthermore, evidence was found for a link between EBV and autoimmune diseases.

### Methods for Production of Non-Recombinant and Recombinant Poxviruses

Methods to obtain recombinant poxviruses such as MVA or to insert exogenous coding sequences into a poxvirus (e.g., MVA) genome are well known to the person skilled in the art. For example, methods for standard molecular biology techniques such as cloning of DNA, DNA and RNA isolation, Western blot analysis, RT-PCR and PCR amplification techniques are described in Molecular Cloning, A laboratory Manual 2nd Ed. (J. Sambrook et al., Cold Spring Harbor Laboratory Press (1989)), and techniques for the handling and manipulation of viruses are described in Virology Methods Manual (B. W. J. Mahy et al. (eds.), Academic Press (1996)). Similarly, techniques and know-how for the handling, manipulation and genetic engineering of poxviruses are described in Molecular Virology: A Practical Approach (A. J. Davison & R. M. Elliott (Eds.), The Practical Approach Series, IRL Press at Oxford University Press, Oxford, UK (1993), see, e.g., Chapter 9: Expression of genes by Vaccinia virus vectors); Current Protocols in Molecular Biology (John Wiley & Son, Inc. (1998), see, e.g., Chapter 16, Section IV: Expression of proteins in mammalian cells using vaccinia viral vector); and Genetic Engineering, Recent Developments in Applications, Apple Academic Press (2011), Dana M. Santos, see, e.g., Chapter 3: Recombinant-mediated Genetic Engineering of a Bacterial Artificial Chromosome Clone of Modified Vaccinia Virus Ankara (MVA)). Construction and isolation of recombinant MVA are also described in Methods and Protocols, Vaccinia Virus and Poxvirology, ISBN 978-1-58829-229-2 (Staib et al.), Humana Press (2004) see, e.g., Chapter 7.

Methods for producing and purifying virus-based material such as viral vectors and/or viruses according to the present invention are known to the person skilled in the art. The methods comprise infection of a suitable cell culture (e.g., Chicken Embryo Fibroblasts (CEF cells) or cell lines such as DF-1, duck, MDCK, quail or chicken derived cell lines, and EB66 cells) and subsequent amplification of the virus under suitable conditions well known to the skilled person. Serum-free cultivation conditions (e.g., medium) as well as serum-containing cultivation methods can be used for virus production, although methods using animal-free material (e.g., the cell culture medium) are preferred. The term “serum-free” medium refers to any cell culture medium that does not contain sera from animal or human origin. As used herein, “animal-free” means any compound or collection of compounds that was not produced in or by an animal cell in a living organism (except for the cell or cell line used for producing and purifying virus-based material). Suitable cell culture media are known to the person skilled in the art. These media comprise salts, vitamins, buffers, energy sources, amino acids and other substances. An example of a medium suitable for serum-free cultivation of CEF cells is medium 199 (Morgan, Morton and Parker; Proc Soc. Exp. Biol. Med. 1950 January; 73(1):1-8; obtainable inter alia from Life Technologies) or VP-SFM (Invitrogen Ltd.) which is preferred. Serum-free methods for virus cultivation and virus amplification in CEF cells are for example described in WO 2004/022729. Upstream and downstream processes for production and purification of virus material are exemplarily described in WO 2012/010280. Further methods useful for purifying viruses of the present application are described in WO 03/054175, WO 07/147528, WO 2008/138533, WO 2009/100521 and WO 2010/130753. Suitable methods for propagation and purification of recombinant poxvirus in duck embryo-derived cell such as but not limited to EB66 cells are described in Leon et al. (Leon et al. (2016), The EB66 cell line as a valuable cell substrate for MVA-based vaccines production, Vaccine 34:5878-5885).

Methods for Production of saRNA

Conventional and synthetic saRNA vaccines are essentially produced in the same manner. Briefly, an mRNA expression plasmid (pDNA) encoding a DNA-dependent RNA polymerase promoter (typically derived from the T7, T3, or SP6 bacteriophages) and the RNA vaccine candidate is designed as a template for in vitro transcription. The flexibility of gene synthesis platforms is a key advantage. For conventional mRNA vaccines the antigenic or immunomodulatory sequence is flanked by 5′ and 3′ untranslated regions (UTRs). A poly(A) tail can either be incorporated from the 3′ end of the pDNA template, or added enzymatically after in vitro transcription. saRNA vaccine pDNA templates contain additional alphavirus replicon genes and conserved sequence elements. The nonstructural proteins 1, 2, 3, and 4 (nsP1-4) are essential for replicon activity as they form the RdRP complex. In vitro transcription is performed on typically on a linearized pDNA template or a linear DNA fragment, typically with a T7 DNA-dependent RNA polymerase, resulting in multiple copies of the RNA transcript. The 5′ end is capped for an efficient translation. This can typically be done by co-transcritional capping with synthetic cap analogues or by post-transcriptional enzymatic capping. After the RNA is capped at the 5′ end and purified, it is ready for formulation and delivery. For saRNA, typically the step of co-transcriptional capping with cap analogues is preferred, as the 5′ cap is different from conventional mRNA.

The RNA product is then undergoing purification which can include steps to remove a by-product of the in vitro transcription in form of double-stranded dsRNA. These can be removed, e.g. by a double-strand specific enzymatic RNase or by chromatography employing material with specific dsRNA affinity. Further chromatographic or other purification steps can be used to increase the purity and quality of the RNA products, e.g. affinity purification, filtration. Affinity purification may also include a polyA-specific resin to enrich the full length and poly-adenylated RNA and remove non-complete shorter by-products.

### Vaccines and Pharmaceutical Compositions

Since the recombinant MVA viruses described herein are highly replication restricted and, thus, highly attenuated, they are ideal candidates for the treatment of a wide range of mammals including humans and even immune-compromised humans. Hence, provided herein are pharmaceutical compositions and vaccines for inducing an immune response in a living animal body, including a human. Additionally provided is a recombinant MVA vector comprising a nucleotide sequence encoding an antigenic determinant of an EBV glycoprotein for use in the treatment and/or prevention of a EBV-caused disease.

The vaccine preferably comprises any of the recombinant MVA viruses described herein formulated in solution in a concentration range of 10⁴ to 10⁹ TCID₅₀/ml, 10⁵ to 5×10⁸ TCID₅₀/ml, 10⁶ to 10⁸ TCID₅₀/ml, or 10⁷ to 10⁸ TCID₅₀/ml. A preferred vaccination dose for humans comprises between 10⁶ to 10⁹ TCID₅₀, including a dose of 10⁶ TCID₅₀, 10⁷ TCID₅₀, or 10⁸ TCID₅₀.

The pharmaceutical compositions provided herein may generally include one or more pharmaceutically acceptable and/or approved carriers, additives, antibiotics, preservatives, adjuvants, diluents and/or stabilizers. Such auxiliary substances can be water, saline, glycerol, ethanol, wetting or emulsifying agents, pH buffering substances, or the like. Suitable carriers are typically large, slowly metabolized molecules such as proteins, polysaccharides, polylactic acids, polyglycolic acids, polymeric amino acids, amino acid copolymers, lipid aggregates, or the like.

For the preparation of vaccines, the recombinant MVA viruses provided herein can be converted into a physiologically acceptable form. This can be done based on experience in the preparation of poxvirus vaccines used for vaccination against smallpox as described by H. Stickl et al., Dtsch. med. Wschr. 99:2386-2392 (1974).

For example, purified viruses can be stored at −80° C. with a titer of 5×10⁸ TCID₅₀/ml formulated in about 10 mM Tris, 140 mM NaCl pH 7.4. For the preparation of vaccine shots, e.g., 10²-10⁸ or 10²-10⁹ particles of the virus can be lyophilized in 100 ml of phosphate-buffered saline (PBS) in the presence of 2% peptone and 1% human albumin in an ampoule, preferably a glass ampoule. Alternatively, the vaccine shots can be produced by stepwise freeze-drying of the virus in a formulation. This formulation can contain additional additives such as mannitol, dextran, sugar, glycine, lactose or polyvinylpyrrolidone or other aids such as antioxidants or inert gas, stabilizers or recombinant proteins (e.g., human serum albumin) suitable for in vivo administration. The glass ampoule is then sealed and can be stored between 4° C. and room temperature for several months. However, as long as no need exists, the ampoule is stored preferably at temperatures below-20° C.

For vaccination or therapy, the lyophilisate can be dissolved in an aqueous solution, preferably physiological saline or Tris buffer, and administered either systemically or locally, i.e., parenteral, subcutaneous, intravenous, intramuscular, intranasal, or any other path of administration known to the skilled practitioner. The mode of administration, the dose and the number of administrations can be optimized by those skilled in the art in a known manner. However, most commonly a patient is vaccinated with a second shot about one month to six weeks after the first vaccination shot.

### Combination Vaccines Using Heterologous Prime-Boost Regimens

The Combination Vaccines and methods described herein may be used as part of a helerologous prime-boost regimen. In the heterologous prime-boost, a first priming vaccination is given followed by one or more subsequent boosting vaccinations.

The MVA and VRP recombinant viral vectors according to the present invention may also be used in heterologous prime-boost regimens in which one or more of the initial prime vaccinations are done with either the MVA or the VRP vector as defined herein and one or more subsequent boosting vaccinations are done with the poxviral vector not used in the prime vaccination, e.g., if a MVA vector defined herein is given in a prime boost, then subsequent boosting vaccinations would be VRP vectors and vice versa.

In a preferred embodiment the prime vaccination is done with the VRP vector and the boosting vaccination with the MVA. Accordingly, one aspect of the invention relates to a combination vaccine comprising:
(a) a first composition comprising an immunologically effective amount of a VRP vector comprising a nucleic acid encoding an antigenic protein, together with a pharmaceutically acceptable carrier; and
(b) a second composition comprising an immunologically effective amount of an MVA vector comprising a nucleic acid encoding an antigenic protein, together with a pharmaceutically acceptable carrier;
   wherein the first compositions is a priming composition and the second composition is a boosting composition, preferably wherein the boosting composition comprises two or more doses of the vector of the boosting composition.
   Vaccines and Kits Comprising Recombinant MVA and saRNA (VRP) Viruses

Also provided herein are vaccines and kits comprising any one or more of the recombinant VRPs and/or MVAs described herein. The kit can comprise one or multiple containers or vials of the recombinant MVA or VRP, together with instructions for the administration of the recombinant MVA and VRP to a subject at risk of an infectious disease. In certain embodiments, the subject is a human. In certain embodiments, the instructions indicate that the recombinant MVA is administered to the subject in a single dose, or in multiple (i.e., 2, 3, 4, etc.) doses. In certain embodiments, the instructions indicate that the recombinant MVA or VRP virus is administered in a first (priming) and second (boosting) administration to naïve or non-naïve subjects. Preferably, a kit comprises at least two vials for prime/boost immunization comprising the recombinant VRPs as described herein for a first inoculation (“priming inoculation”) in a first vial/container and for an at least second and/or third and/or further inoculation (“boosting inoculation”) in a second and/or further vial/container comprising the recombinant MVA.

Other embodiments of the invention will be apparent to those skilled in the art from consideration of the specification and practice of the invention disclosed herein. It is intended that the specification and examples be considered as exemplary only, with a true scope and spirit of the invention being indicated by the appended claims.

### Embodiments

Embodiment 1 is a vaccine combination comprising
(a) a first composition comprising an immunologically effective amount of a saRNA comprising a nucleic acid encoding an antigenic protein, together with a pharmaceutically acceptable carrier; and
(b) a second composition comprising an immunologically effective amount of an MVA vector comprising a nucleic acid encoding an antigenic protein, together with a pharmaceutically acceptable carrier;
wherein one of the compositions is a priming composition and the other composition is a boosting composition.

Embodiment 2 is a vaccine combination according to embodiment 1, wherein the first composition is used for priming an immune response and the second composition is used for boosting said immune response.

Embodiment 3 is a vaccine combination according to embodiment 1, wherein the second composition is used for priming an immune response and the first composition is used for boosting said immune response.

Embodiment 4 is a vaccine combination according to any one of embodiments 1-3, wherein the antigenic protein is an infectious disease antigen or a tumor-associated antigen.

Embodiment 5 is a vaccine combination according to embodiment 4, wherein the antigenic protein is an infectious disease antigen.

Embodiment 6 is a vaccine combination according to embodiment 5, wherein the antigenic protein is a viral antigen, a bacterial antigen, a fungal antigen, or a parasite antigen.

Embodiment 7 is a vaccine combination according to embodiment 6, wherein the antigenic protein is a viral antigen.

Embodiment 8 is a vaccine combination according to embodiment 7, wherein the viral antigen is derived from a virus selected from the group consisting of adenovirus, Arbovirus, Astrovirus, Coronavirus, Coxsackievirus, Crimean-Congo hemorrhagic fever virus, cytomegalovirus (“CMV”), dengue virus, Ebola virus, Epstein-Barr virus (“EBV”), Foot-and-mouth disease virus, Guanarito virus, Hendra virus, herpes simplex virus-type 1 (“HSV-1”), herpes simplex virus-type 2 (“HSV-2”), human herpesvirus-type 6 (“HHV-6”), human herpesvirus-type 8 (“HHV-8”), hepatitis A virus (“HAV”), hepatitis B virus (“HBV”), hepatitis C virus (“HCV”), hepatitis D virus (“HDV”), hepatitis E virus (“HEV”), human immunodeficiency virus (“HIV”), influenza virus, Japanese encephalitis virus, Junin virus, Lassa virus, Machupo virus, Marburg virus, measles virus, human metapneumovirus, Molluscum contagiosum virus, mumps virus, Newcastle disease virus, Nipha virus, Norovirus, Norwalk virus, human papillomavirus (“HPV”), parainfluenza virus, parvovirus, poliovirus, rabies virus, respiratory syncytial virus (“RSV”), rhinovirus, rotavirus, rubella virus, Sabia virus, severe acute respiratory syndrome virus (“SARS”), varicella zoster virus, variola virus, West Nile virus, and yellow fever virus.

Embodiment 9 is a vaccine combination according to embodiment 6, wherein the antigenic protein is a bacterial antigen.

Embodiment 10 is a vaccine combination according to embodiment 9, wherein the bacterial antigen is derived from a bacterium selected from the group consisting of Bacillus anthracis, Bordetella pertussis, Borrelia burgdorferi, Brucella abortus, Brucella canis, Brucella melitensis, Brucella suis, Burkholderia mallei, Burkholderia pseudomallei, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diptheriae, Enterococcus faecalis, Enterococcus faecium, Escherichia coli, enterotoxigenic Escherichia coli, enteropathogenic Escherichia coli, Escherichia coli) 157: H7, Francisella tularensis, Haemophilus influenza, Helicobacter pylori, Legionella pneumophila, Leptospira interrogans, Listeria monocytogenes, Mycobacterium leprae, Mycobacterium tuberculosis, Mycoplasma pneumoniae, Neisseria gonorrhoeae, Neisseria meningitides, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Vibrio cholerae, and Yersinia pestis.

Embodiment 11 is a vaccine combination according to embodiment 6, wherein the infectious disease antigen is a fungal antigen.

Embodiment 12 is a vaccine combination according to embodiment 11, wherein the fungal antigen is derived from a fungus selected from the group consisting of Aspergillus clavatus, Aspergillus flavus, Aspergillus fumigatus, Aspergillus nidulans, Aspergillus niger, Aspergillus terreus, Blastomyces dermatitidis, Candida albicans, Candida dubliniensis, Candida glabrata, Candida parapsilosis, Candida rugosa, Candida tropicalis, Cryptococcus albidus, Cryptococcus gattii, Cryptococcus laurentii, Cryptococcus neoformans, Histoplasma capsulatum, Microsporum canis, Pneumocystis carinii, Pneumocystis jirovecii, Sporothrix schenckii, Stachbotrys chartarum, Tinea barbae, Tinea captitis, Tinea corporis, Tinea cruris, Tinea faciei, Tinea incognito, Tinea nigra, Tinea versicolor, Trichophyton rubrum and Trichophyton tonsurans.

Embodiment 13 is a vaccine combination according to embodiment 6, wherein the antigenic protein is a parasite antigen.

Embodiment 14 is a vaccine combination according to embodiment 13, wherein the parasite antigen is derived from a parasite selected from the group consisting of Anisakis spp. Babesia spp., Baylisascaris procyonis, Cryptosporidium spp., Cyclospora cayetanensis, Diphyllobothrium spp., Dracunculus medinensis, Entamoeba histolytica, Giardia duodenalis, Giardia intestinalis, Giardia lamblia, Leishmania sp., Plasmodium falciparum, Schistosoma mansoni, Schistosoma haematobium, Schistosoma japonicum, Taenia spp., Toxoplasma gondii, Trichinella spiralis, and Trypanosoma cruzi.

Embodiment 15 is a vaccine combination according to embodiment 4, wherein the antigenic protein is a tumor-associated antigen.

Embodiment 16 is a vaccine combination according to embodiment 15, wherein the tumor-associated antigen is selected from the group consisting of 5-α-reductase, α-fetoprotein (“AFP”), AM-1, APC, April, B melanoma antigen gene (“BAGE”), β-catenin, Bcl12, bcr-abl, Brachyury, CA-125, caspase-8 (“CASP-8”, also known as “FLICE”), Cathepsins, CD19, CD20, CD21/complement receptor 2 (“CR2”), CD22/BL-CAM, CD23/FcERII, CD33, CD35/complement receptor 1 (“CR1”), CD44/PGP-1, CD45/leucocyte common antigen (“LCA”), CD46/membrane cofactor protein (“MCP”), CD52/CAMPATH-1, CD55/decay accelerating factor (“DAF”), CD59/protectin, CDC27, CDK4, carcinoembryonic antigen (“CEA”), c-myc, cyclooxygenase-2 (“cox-2”), deleted in colorectal cancer gene (“DCC”), DcR3, E6/E7, CGFR, EMBP, Dna78, farnesyl transferase, fibroblast growth factor-8a (“FGF8a”), fibroblast growth factor-8b (“FGF8b”), FLK-1/KDR, folic acid receptor, G250, G melanoma antigen gene family (“GAGE-family”), gastrin 17, gastrin-releasing hormone, ganglioside 2 (“GD2”)/ganglioside 3 (“GD3”)/ganglioside-monosialic acid-2 (“GM2”), gonadotropin releasing hormone (“GnRH”), UDP-GlcNAc:R_{1l Man(α}1-6)R₂ [GlcNAc to Man(α1-6)] β1,6-N-acetylglucosaminyltransferase V (“GnT V”), GP1, gp100/Pme117, gp-100-in4, gp15, gp75/tyrosine-related protein-1 (“gp75/TRP-1”), human chorionic gonadotropin (“hCG”), heparanase, Her2/neu, human mammary tumor virus (“HMTV”), 70 kiloDalton heat-shock protein (“HSP70”), human telomerase reverse transcriptase (“hTERT”), insulin-like growth factor receptor-1 (“IGFR-1”), interleukin-13 receptor (“IL-13R”), inducible nitric oxide synthase (“iNOS”), Ki67, KIAA0205, K-ras, H-ras, N-ras, KSA, LKLR-FUT, melanoma antigen-encoding gene 1 (“MAGE-1”), melanoma antigen-encoding gene 2 (“MAGE-2”), melanoma antigen-encoding gene 3 (“MAGE-3”), melanoma antigen-encoding gene 4 (“MAGE-4”), mammaglobin, MAP17, Melan-A/melanoma antigen recognized by T-cells-1 (“MART-1”), mesothelin, MIC A/B, MT-MMPs, mucin, testes-specific antigen NY-ESO-1, osteonectin, p15, P170/MDR1, p53, p97/melanotransferrin, PAI-1, platelet-derived growth factor (“PDGF”), uPA, PRAME, probasin, progenipoietin, prostate-specific antigen (“PSA”), prostate-specific membrane antigen (“PSMA”), prostatic acid phosphatase (“PAP”), RAGE-1, Rb, RCAS1, SART-1, SSX-family, STAT3, STn, TAG-72, transforming growth factor-alpha (“TGF-α”), transforming growth factor-beta (“TGF-β”), Thymosin-beta-15, tumor necrosis factor-alpha (“TNF-α”), TP1, TRP-2, tyrosinase, vascular endothelial growth factor (“VEGF”), ZAG, p16INK4, and glutathione-S-transferase (“GST”).

Embodiment 17 is a vaccine combination according to any one of embodiments 1-8, wherein the antigenic proteins are any of the structural and non-structural proteins of EBV.

Embodiment 18 is a vaccine combination according to embodiment 17, wherein the antigenic proteins are selected from gp350, gH, gL, EBNA3A, and BRLF1/BZLF1 fusion.

Embodiment 19 is a vaccine combination according to embodiment 18, wherein the antigenic proteins are encoded by SEQ ID NO: 1, SEQ ID NO: 2, SEQ ID NO: 3, SEQ ID NO: 5 and SEQ ID NO: 4.

Embodiment 20 is a vaccine combination according to any one of embodiments 1-19, wherein the saRNA is a VRP, preferably VEEV, more preferably TC83.

Embodiment 21 is a vaccine combination according to any one of embodiments 1-19, wherein the MVA is MVA-BN.

Embodiment 22 is a vaccine combination according to embodiment 20, wherein the VRP in the first composition comprises a nucleic acid encoding an antigenic protein selected from the group consisting of gp350, gH and gL.

Embodiment 23 is a vaccine combination according to embodiment 22, wherein the antigenic proteins are encoded by SEQ ID NO: 1, SEQ ID NO: 2, and SEQ ID NO: 3.

Embodiment 24 is a vaccine combination according to any one of embodiments 1-23 for use in generating a protective immune response against an infectious disease or a tumor-associated disease, wherein the first composition is used for priming said immune response and the second composition is used for boosting said immune response.

Embodiment 25 is a vaccine combination according to any one of embodiments 1-23 for use in generating a protective immune response against an infectious disease or a tumor-associated disease, wherein the second composition is used for priming said immune response and the first composition is used for boosting said immune response.

Embodiment 26 is a vaccine combination according to any one of embodiments 1-25, wherein the boosting composition comprises two or more doses of the vector of the boosting composition.

Embodiment 27 is a kit comprising:
(a) a first composition comprising an immunologically effective amount of a saRNA comprising a nucleic acid encoding an antigenic protein, together with a pharmaceutically acceptable carrier; and
(b) a second composition comprising an immunologically effective amount of a MVA vector comprising a nucleic acid encoding an antigenic protein, together with a pharmaceutically acceptable carrier;
wherein one of the compositions is a priming composition and the other composition is a boosting composition.

Embodiment 28 is a kit according to embodiment 27, wherein the first composition is used for priming an immune response and the second composition is used for boosting said immune response.

Embodiment 29 is a kit according to embodiment 27, wherein the second composition is used for priming an immune response and the first composition is used for boosting said immune response.

Embodiment 30 is a kit according to any one of embodiments 27-29, wherein the antigenic protein is an infectious disease antigen or a tumor-associated antigen.

Embodiment 31 is a kit according to embodiment 30, wherein the disease-associated antigen is an infectious disease antigen.

Embodiment 32 is a kit according to embodiment 31, wherein the infectious disease antigen is a viral antigen, a bacterial antigen, a fungal antigen, or a parasite antigen.

Embodiment 33 is a kit according to embodiment 32, wherein the infectious disease antigen is a viral antigen.

Embodiment 34 is a kit according to embodiment 33, wherein the viral antigen is derived from a virus selected from the group consisting of adenovirus, Arbovirus, Astrovirus, Coronavirus, Coxsackievirus, Crimean-Congo hemorrhagic fever virus, cytomegalovirus (“CMV”), dengue virus, Ebola virus, Epstein-Barr virus (“EBV”), Foot-and-mouth disease virus, Guanarito virus, Hendra virus, herpes simplex virus-type 1 (“HSV-1”), herpes simplex virus-type 2 (“HSV-2”), human herpesvirus-type 6 (“HHV-6”), human herpesvirus-type 8 (“HHV-8”), hepatitis A virus (“HAV”), hepatitis B virus (“HBV”), hepatitis C virus (“HCV”), hepatitis D virus (“HDV”), hepatitis E virus (“HEV”), human immunodeficiency virus (“HIV”), influenza virus, Japanese encephalitis virus, Junin virus, Lassa virus, Machupo virus, Marburg virus, measles virus, human metapneumovirus, Molluscum contagiosum virus, mumps virus, Newcastle disease virus, Nipha virus, Norovirus, Norwalk virus, human papillomavirus (“HPV”), parainfluenza virus, parvovirus, poliovirus, rabies virus, respiratory syncytial virus (“RSV”), rhinovirus, rotavirus, rubella virus, Sabia virus, severe acute respiratory syndrome virus (“SARS”), varicella zoster virus, variola virus, West Nile virus, and yellow fever virus.

Embodiment 35 is a kit according to embodiment 32, wherein the infectious disease antigen is a bacterial antigen.

Embodiment 36 is a kit according to embodiment 35, wherein the bacterial antigen is derived from a bacterium selected from the group consisting of Bacillus anthracis, Bordetella pertussis, Borrelia burgdorferi, Brucella abortus, Brucella canis, Brucella melitensis, Brucella suis, Burkholderia mallei, Burkholderia pseudomallei, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diptheriae, Enterococcus faecalis, Enterococcus faecium, Escherichia coli, enterotoxigenic Escherichia coli, enteropathogenic Escherichia coli, Escherichia coli) 157: H7, Francisella tularensis, Haemophilus influenza, Helicobacter pylori, Legionella pneumophila, Leptospira interrogans, Listeria monocytogenes, Mycobacterium leprae, Mycobacterium tuberculosis, Mycoplasma pneumoniae, Neisseria gonorrhoeae, Neisseria meningitides, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Vibrio cholerae, and Yersinia pestis.

Embodiment 37 is a kit according to embodiment 32, wherein the infectious disease antigen is a fungal antigen.

Embodiment 38 is a kit according to embodiment 37, wherein the fungal antigen is derived from a fungus selected from the group consisting of Aspergillus clavatus, Aspergillus flavus, Aspergillus fumigatus, Aspergillus nidulans, Aspergillus niger, Aspergillus terreus, Blastomyces dermatitidis, Candida albicans, Candida dubliniensis, Candida glabrata, Candida parapsilosis, Candida rugosa, Candida tropicalis, Cryptococcus albidus, Cryptococcus gattii, Cryptococcus laurentii, Cryptococcus neoformans, Histoplasma capsulatum, Microsporum canis, Pneumocystis carinii, Pneumocystis jirovecii, Sporothrix schenckii, Stachbotrys chartarum, Tinea barbae, Tinea captitis, Tinea corporis, Tinea cruris, Tinea faciei, Tinea incognito, Tinea nigra, Tinea versicolor, Trichophyton rubrum and Trichophyton tonsurans.

Embodiment 39 is a kit according to embodiment 32, wherein the infectious disease antigen is a parasite antigen.

Embodiment 40 is a kit according to embodiment 39, wherein the parasite antigen is derived from a parasite selected from the group consisting of Anisakis spp. Babesia spp., Baylisascaris procyonis, Cryptosporidium spp., Cyclospora cayetanensis, Diphyllobothrium spp., Dracunculus medinensis, Entamoeba histolytica, Giardia duodenalis, Giardia intestinalis, Giardia lamblia, Leishmania sp., Plasmodium falciparum, Schistosoma mansoni, Schistosoma haematobium, Schistosoma japonicum, Taenia spp., Toxoplasma gondii, Trichinella spiralis, and Trypanosoma cruzi.

Embodiment 41 is a kit according to embodiment 30, wherein the disease-associated antigen is a tumor-associated antigen.

Embodiment 42 is a kit according to embodiment 41, wherein the tumor-associated antigen is selected from the group consisting of 5-α-reductase, α-fetoprotein (“AFP”), AM-1, APC, April, B melanoma antigen gene (“BAGE”), β-catenin, Bcl12, bcr-abl, Brachyury, CA-125, caspase-8 (“CASP-8”, also known as “FLICE”), Cathepsins, CD19, CD20, CD21/complement receptor 2 (“CR2”), CD22/BL-CAM, CD23/FcERII, CD33, CD35/complement receptor 1 (“CR1”), CD44/PGP-1, CD45/leucocyte common antigen (“LCA”), CD46/membrane cofactor protein (“MCP”), CD52/CAMPATH-1, CD55/decay accelerating factor (“DAF”), CD59/protectin, CDC27, CDK4, carcinoembryonic antigen (“CEA”), c-myc, cyclooxygenase-2 (“cox-2”), deleted in colorectal cancer gene (“DCC”), DcR3, E6/E7, CGFR, EMBP, Dna78, farnesyl transferase, fibroblast growth factor-8a (“FGF8a”), fibroblast growth factor-8b (“FGF8b”), FLK-1/KDR, folic acid receptor, G250, G melanoma antigen gene family (“GAGE-family”), gastrin 17, gastrin-releasing hormone, ganglioside 2 (“GD2”)/ganglioside 3 (“GD3”)/ganglioside-monosialic acid-2 (“GM2”), gonadotropin releasing hormone (“GnRH”), UDP-GlcNAc:R₁Man(α1-6)R₂ [GlcNAc to Man(α1-6)] β1,6-N-acetylglucosaminyltransferase V (“GnT V”), GP1, gp100/Pme117, gp-100-in4, gp15, gp75/tyrosine-related protein-1 (“gp75/TRP-1”), human chorionic gonadotropin (“hCG”), heparanase, Her2/neu, human mammary tumor virus (“HMTV”), 70 kiloDalton heat-shock protein (“HSP70”), human telomerase reverse transcriptase (“hTERT”), insulin-like growth factor receptor-1 (“IGFR-1”), interleukin-13 receptor (“IL-13R”), inducible nitric oxide synthase (“iNOS”), Ki67, KIAA0205, K-ras, H-ras, N-ras, KSA, LKLR-FUT, melanoma antigen-encoding gene 1 (“MAGE-1”), melanoma antigen-encoding gene 2 (“MAGE-2”), melanoma antigen-encoding gene 3 (“MAGE-3”), melanoma antigen-encoding gene 4 (“MAGE-4”), mammaglobin, MAP17, Melan-A/melanoma antigen recognized by T-cells-1 (“MART-1”), mesothelin, MIC A/B, MT-MMPs, mucin, testes-specific antigen NY-ESO-1, osteonectin, p15, P170/MDR1, p53, p97/melanotransferrin, PAI-1, platelet-derived growth factor (“PDGF”), uPA, PRAME, probasin, progenipoietin, prostate-specific antigen (“PSA”), prostate-specific membrane antigen (“PSMA”), prostatic acid phosphatase (“PAP”), RAGE-1, Rb, RCAS1, SART-1, SSX-family, STAT3, STn, TAG-72, transforming growth factor-alpha (“TGF-α”), transforming growth factor-beta (“TGF-β”), Thymosin-beta-15, tumor necrosis factor-alpha (“TNF-α”), TP1, TRP-2, tyrosinase, vascular endothelial growth factor (“VEGF”), ZAG, p16INK4, and glutathione-S-transferase (“GST”).

Embodiment 43 is a kit according to any one of embodiments 27-34, wherein the antigenic protein is any of the structural and non-structural of EBV.

Embodiment 44 is a kit according to embodiment 43, wherein the antigenic proteins are selected from gp350, gH, gL, EBNA3A, and BRLF1/BZLF1 fusion.

Embodiment 45 is a kit according to embodiment 44, wherein the antigenic proteins are encoded by SEQ ID NO: 1, SEQ ID NO: 2, SEQ ID NO: 3, SEQ ID NO: 5 and SEQ ID NO: 4.

Embodiment 46 is a kit according to any one of embodiments 27-45, wherein the saRNA is a VRP, preferably VEEV, more preferably TC83.

Embodiment 47 is a kit according to any one of embodiments 27-45, wherein the MVA is MVA-BN.

Embodiment 48 is a kit according to embodiment 46, wherein the VRP in the first composition comprises a nucleic acid encoding an antigenic protein selected from the group consisting of gp350, gH and gL.

Embodiment 49 is a kit according to embodiment 48, wherein the antigenic proteins are encoded by SEQ ID NO: 1, SEQ ID NO: 2, and SEQ ID NO: 3.

Embodiment 50 is a kit according to any one of embodiments 27-49, for use in generating a protective immune response against an infectious disease or a tumor-associated disease, wherein the first composition is used for priming said immune response and the second composition is used for boosting said immune response.

Embodiment 51 is a kit according to any one of embodiments 27-49, for use in generating a protective immune response against an infectious disease or a tumor-associated disease, wherein the second composition is used for priming said immune response and the first composition is used for boosting said immune response.

Embodiment 52 is a kit according to any one of embodiments 1-51, wherein the boosting composition comprises two or more doses of the vector of the boosting composition.

Embodiment 53 is a vaccine combination according to any one of embodiments 1-23, the vaccine combination for use according to any one of embodiments 24-26, the kit according to any one of embodiments 27-49, the kit for use according to any one of embodiments 50-52, wherein the MVA used for generating the recombinant virus is a MVA-BN virus or a derivative having the capability of reproductive replication in vitro in chicken embryo fibroblasts (CEF) cells, but no capability of reproductive replication in the human keratinocyte cell line HaCat, the human bone osteosarcoma cell line 143B, the human embryo kidney cell line 293, and the human cervix adenocarcinoma cell line HeLa.

Embodiment 54 is a vaccine combination according to any one of embodiments 1-23, the vaccine combination for use according to any one of embodiments 24-26, the kit according to any one of embodiments 27-49, the kit for use according to any one of embodiments 50-52, wherein the MVA used for generating the recombinant virus is MVA-BN as deposited at the European Collection of Animal Cell cultures (ECACC) under accession number V00083008.

Embodiment 55 is a use of the vaccine combination according to any one of embodiments 1-23 or the kit according to any one of embodiments 27-49 for manufacturing a pharmaceutical composition or medicament for treatment and/or prevention of an infectious disease.

Embodiment 56 is a pharmaceutical composition comprising the vaccine combination according to embodiments 1-23 and a pharmaceutically acceptable carrier, diluent and/or additive.

Embodiment 57 is a method of inducing an immune response against a virus in a subject, the method comprising administering to the subject:
(a) a first composition comprising an immunologically effective amount of a saRNA comprising a nucleic acid encoding antigenic proteins, together with a pharmaceutically acceptable carrier; and
(b) a second composition comprising an immunologically effective amount of a MVA vector comprising a nucleic acid encoding an antigenic protein, together with a pharmaceutically acceptable carrier;
wherein one of the compositions is a priming composition and the other composition is a boosting composition.

Embodiment 58 is a method according to embodiment 57, wherein the first composition is used for priming an immune response and the second composition is used for boosting said immune response.

Embodiment 59 is a method according to embodiment 57, wherein the second composition is used for priming an immune response and the first composition is used for boosting said immune response.

Embodiment 60 is a method according to any one of embodiments 57-59, wherein the disease-associated antigen is an infectious disease antigen or a tumor-associated antigen.

Embodiment 61 is a method according to embodiment 60, wherein the disease-associated antigen is an infectious disease antigen.

Embodiment 62 is a method according to embodiment 61, wherein the infectious disease antigen is a viral antigen, a bacterial antigen, a fungal antigen, or a parasite antigen.

Embodiment 63 is a method according to embodiment 62, wherein the infectious disease antigen is a viral antigen.

Embodiment 64 is a method according to embodiment 63, wherein the viral antigen is derived from a virus selected from the group consisting of adenovirus, Arbovirus, Astrovirus, Coronavirus, Coxsackievirus, Crimean-Congo hemorrhagic fever virus, cytomegalovirus (“CMV”), dengue virus, Ebola virus, Epstein-Barr virus (“EBV”), Foot-and-mouth disease virus, Guanarito virus, Hendra virus, herpes simplex virus-type 1 (“HSV-1”), herpes simplex virus-type 2 (“HSV-2”), human herpesvirus-type 6 (“HHV-6”), human herpesvirus-type 8 (“HHV-8”), hepatitis A virus (“HAV”), hepatitis B virus (“HBV”), hepatitis C virus (“HCV”), hepatitis D virus (“HDV”), hepatitis E virus (“HEV”), human immunodeficiency virus (“HIV”), influenza virus, Japanese encephalitis virus, Junin virus, Lassa virus, Machupo virus, Marburg virus, measles virus, human metapneumovirus, Molluscum contagiosum virus, mumps virus, Newcastle disease virus, Nipha virus, Norovirus, Norwalk virus, human papillomavirus (“HPV”), parainfluenza virus, parvovirus, poliovirus, rabies virus, respiratory syncytial virus (“RSV”), rhinovirus, rotavirus, rubella virus, Sabia virus, severe acute respiratory syndrome virus (“SARS”), varicella zoster virus, variola virus, West Nile virus, and yellow fever virus.

Embodiment 65 is a method according to embodiment 62, wherein the infectious disease antigen is a bacterial antigen.

Embodiment 66 is a method according to embodiment 65, wherein the bacterial antigen is derived from a bacterium selected from the group consisting of Bacillus anthracis, Bordetella pertussis, Borrelia burgdorferi, Brucella abortus, Brucella canis, Brucella melitensis, Brucella suis, Burkholderia mallei, Burkholderia pseudomallei, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diptheriae, Enterococcus faecalis, Enterococcus faecium, Escherichia coli, enterotoxigenic Escherichia coli, enteropathogenic Escherichia coli, Escherichia coli) 157: H7, Francisella tularensis, Haemophilus influenza, Helicobacter pylori, Legionella pneumophila, Leptospira interrogans, Listeria monocytogenes, Mycobacterium leprae, Mycobacterium tuberculosis, Mycoplasma pneumoniae, Neisseria gonorrhoeae, Neisseria meningitides, Pseudomonas aeruginosa, Rickettsia rickettsia, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Vibrio cholerae, and Yersinia pestis.

Embodiment 67 is a method according to embodiment 62, wherein the infectious disease antigen is a fungal antigen.

Embodiment 68 is a method according to embodiment 67, wherein the fungal antigen is derived from a fungus selected from the group consisting of Aspergillus clavatus, Aspergillus flavus, Aspergillus fumigatus, Aspergillus nidulans, Aspergillus niger, Aspergillus terreus, Blastomyces dermatitidis, Candida albicans, Candida dubliniensis, Candida glabrata, Candida parapsilosis, Candida rugosa, Candida tropicalis, Cryptococcus albidus, Cryptococcus gattii, Cryptococcus laurentii, Cryptococcus neoformans, Histoplasma capsulatum, Microsporum canis, Pneumocystis carinii, Pneumocystis jirovecii, Sporothrix schenckii, Stachbotrys chartarum, Tinea barbae, Tinea captitis, Tinea corporis, Tinea cruris, Tinea faciei, Tinea incognito, Tinea nigra, Tinea versicolor, Trichophyton rubrum and Trichophyton tonsurans.

Embodiment 69 is a method according to embodiment 62, wherein the infectious disease antigen is a parasite antigen.

Embodiment 70 is a method according to embodiment 69, wherein the parasite antigen is derived from a parasite selected from the group consisting of Anisakis spp. Babesia spp., Baylisascaris procyonis, Cryptosporidium spp., Cyclospora cayetanensis, Diphyllobothrium spp., Dracunculus medinensis, Entamoeba histolytica, Giardia duodenalis, Giardia intestinalis, Giardia lamblia, Leishmania sp., Plasmodium falciparum, Schistosoma mansoni, Schistosoma haematobium, Schistosoma japonicum, Taenia spp., Toxoplasma gondii, Trichinella spiralis, and Trypanosoma cruzi.

Embodiment 71 is a method according to embodiment 60, wherein the disease-associated antigen is a tumor-associated antigen.

Embodiment 72 is a method according to embodiment 71, wherein the tumor-associated antigen is selected from the group consisting of 5-α-reductase, α-fetoprotein (“AFP”), AM-1, APC, April, B melanoma antigen gene (“BAGE”), β-catenin, Bcl12, bcr-abl, Brachyury, CA-125, caspase-8 (“CASP-8”, also known as “FLICE”), Cathepsins, CD19, CD20, CD21/complement receptor 2 (“CR2”), CD22/BL-CAM, CD23/FcERII, CD33, CD35/complement receptor 1 (“CR1”), CD44/PGP-1, CD45/leucocyte common antigen (“LCA”), CD46/membrane cofactor protein (“MCP”), CD52/CAMPATH-1, CD55/decay accelerating factor (“DAF”), CD59/protectin, CDC27, CDK4, carcinoembryonic antigen (“CEA”), c-myc, cyclooxygenase-2 (“cox-2”), deleted in colorectal cancer gene (“DCC”), DcR3, E6/E7, CGFR, EMBP, Dna78, farnesyl transferase, fibroblast growth factor-8a (“FGF8a”), fibroblast growth factor-8b (“FGF8b”), FLK-1/KDR, folic acid receptor, G250, G melanoma antigen gene family (“GAGE-family”), gastrin 17, gastrin-releasing hormone, ganglioside 2 (“GD2”)/ganglioside 3 (“GD3”)/ganglioside-monosialic acid-2 (“GM2”), gonadotropin releasing hormone (“GnRH”), UDP-GlcNAc:R₁Man(α1-6)R₂ [GlcNAc to Man(α1-6)] β1,6-N-acetylglucosaminyltransferase V (“GnT V”), GP1, gp100/Pme117, gp-100-in4, gp15, gp75/tyrosine-related protein-1 (“gp75/TRP-1”), human chorionic gonadotropin (“hCG”), heparanase, Her2/neu, human mammary tumor virus (“HMTV”), 70 kiloDalton heat-shock protein (“HSP70”), human telomerase reverse transcriptase (“hTERT”), insulin-like growth factor receptor-1 (“IGFR-1”), interleukin-13 receptor (“IL-13R”), inducible nitric oxide synthase (“iNOS”), Ki67, KIAA0205, K-ras, H-ras, N-ras, KSA, LKLR-FUT, melanoma antigen-encoding gene 1 (“MAGE-1”), melanoma antigen-encoding gene 2 (“MAGE-2”), melanoma antigen-encoding gene 3 (“MAGE-3”), melanoma antigen-encoding gene 4 (“MAGE-4”), mammaglobin, MAP17, Melan-A/melanoma antigen recognized by T-cells-1 (“MART-1”), mesothelin, MIC A/B, MT-MMPs, mucin, testes-specific antigen NY-ESO-1, osteonectin, p15, P170/MDR1, p53, p97/melanotransferrin, PAI-1, platelet-derived growth factor (“PDGF”), uPA, PRAME, probasin, progenipoietin, prostate-specific antigen (“PSA”), prostate-specific membrane antigen (“PSMA”), prostatic acid phosphatase (“PAP”), RAGE-1, Rb, RCAS1, SART-1, SSX-family, STAT3, STn, TAG-72, transforming growth factor-alpha (“TGF-α”), transforming growth factor-beta (“TGF-β”), Thymosin-beta-15, tumor necrosis factor-alpha (“TNF-α”), TP1, TRP-2, tyrosinase, vascular endothelial growth factor (“VEGF”), ZAG, p16INK4, and glutathione-S-transferase (“GST”).

Embodiment 73 is a method according to any one of embodiments 57-64, wherein the antigenic protein is any of the structural and non-structural of EBV.

Embodiment 74 is a method according to embodiment 73, wherein the antigenic proteins are selected from gp350, gH, gL, EBNA3A, and BRLF1/BZLF1 fusion.

Embodiment 75 is a method according to embodiment 74, wherein the antigenic proteins are encoded by SEQ ID NO: 1, SEQ ID NO: 2, SEQ ID NO: 3, SEQ ID NO: 5 and SEQ ID NO: 4.

Embodiment 76 is a method according to any one of embodiments 57-75, wherein saRNA is a VRP, preferably VEEV, more preferably TC83.

Embodiment 77 is a method according to embodiments 57-75, wherein the MVA is MVA-BN.

Embodiment 78 is a method according to embodiment 76, wherein the VRP in the first composition comprises a nucleic acid encoding an antigenic protein selected from the group consisting of gp350, gH and gL.

Embodiment 79 is a method according to embodiment 78, wherein the antigenic proteins are encoded by SEQ ID NO: 1, SEQ ID NO: 2, and SEQ ID NO: 3.

Embodiment 80 is a method according to any one of embodiments 57-79, wherein the boosting composition is administered 1-12 weeks after administering the priming composition.

Embodiment 81 is a method according to any one of embodiments 57-81, wherein the boosting composition is administered two or more times to the subject.

Reference will now be made in detail to exemplary embodiments of the invention, examples of which are illustrated in the accompanying drawings.

### EXAMPLES

The detailed examples which follow are intended to contribute to a better understanding of the present invention. However, the invention is not limited by the examples. Other embodiments of the invention will be apparent to those skilled in the art from consideration of the specification and practice of the invention disclosed herein.

### Example 1: Material and Methods

### Construction of Recombinant MVA, VRP (Alphavirus Replicon Particles), and Adenovirus

The following sections describe construction of recombinant MVAs, Ads, and VRPs comprising one or more heterologous nucleic acids expressing an antigenic determinant of an Epstein-Barr virus EBV glycoprotein and/or a further EBV protein. All other constructs described herein are made using similar methods.

### Construction of MVA-mBN443B (MVA-BN-EBV)

MVA-BN-EBV (MVA-mBN443B) is based on the Modified Vaccinia Ankara-Bavarian Nordic (MVA-BN®) vector and encodes the non-structural proteins EBNA3A and a fusion of the early trans activators BRLF1/BZLF1 as well as the structural glycoproteins (gp) gH and gL and a truncated soluble form of gp350 (amino acids 2-434) with a flexible linker and a GCN4 multimerization domain for multimer formation. The nucleotide sequences were codon optimized and synthesized including promoter sequences and sequences necessary for cloning. The individual EBV genes were cloned in transfer plasmids with sequences homologous to the surrounding MVA sequences if the respective insertion sites, the intergenic regions (IGR 44/45 and IGR 88/89), targeted for insertion via homologous recombination.

### Construction of VRP-BN011 (Alphavirus Replicon Particles Based Recombinant Vaccine)

The recombinant alphavirus replicon particle VRP-BN011 is a multivalent recombinant VRP encoding three EBV antigens. VRP-BN011 consists of a replicon derived from the VEEV TC83 attenuated strain that expresses a soluble truncated gp350 (amino acids 2-434) with a GCN4 multimerization domain for multimer formation, a gH and a gL protein. The basis is the sequence of the TC83 vaccine strain of VEEV, where the VEEV virus shell cap-env polyprotein sequence is deleted and replaced by the listed genes of EBV. The three EBV genes are encoded on a polyprotein with 2A peptides between the individual sequences. The T2A and P2A peptide sequences are inserted between the three EBV coding sequences (EBV gp350-GCN4 and gH without stop codons) to generate separate proteins inducing a missing peptide link in the prolonging protein chain. Further there is a furin cleavage site inserted just downstream of the gH coding sequence to cut off the P2A sequence from the C-terminus of the maturating gH protein. The sequences were codon optimized and synthesized including sequences necessary for cloning the VRP stocks were prepared by transfecting HEK293T cells with three plasmids (the CMV promoter driven packaging plasmids, coding for cap or for env and the CMV launched recombinant replicon with the EBV genes on a plasmid). Supernatants were harvested and applied to sucrose cushion purification. The VRP stocks were titrated in several dilution steps on Vero cells by infection and subsequentstaining of the double stranded RNA dsRNA replication intermediates with a dsRNA specific mouse monoclonal antibody (J2, Jena Bioscience) and analysed by FACS.

### Construction of AdC68gp350 (Adenovirus Vector Based Recombinant Vaccine)

The adenovirus vector (AdC68) expresses a truncated soluble form of the EBV gp350 protein with a flexible linker and a multimerization domain for multimer formation (AdC68gp350).

### Multiplex Enzyme-linked Immunosorbent Assay (ELISA) by Luminex

IgG antibodies specific for the EBV antigens gp350, gH/gL/gp42 complex and gH are quantified in NHP serum samples by Multiplex ELISA. For that purpose, Luminex Magnetic Microspheres are coupled to either gp350 protein, gH/gL complex or gH protein according to manufacturer's instruction (xMAP® Antibody Coupling Kit, Luminex). Since each microsphere is uniquely addressable, multiple immunoassays can be performed simultaneously with the same sample, in the same well. Briefly, to detect NHP IgG antibodies specific for the EBV antigens gp350, gH/gL/gp42 complex and gH serum is incubated with the antigen-coupled microspheres. Different serum dilutions are tested in accordance with the expected antibody levels (1:200-1:20000). In addition, a standard is included into the analysis (Human serum, purchased from BiolVT, lot #BRH1079042). After washing steps, the detection reagent (Detection antibody: Goat F(ab′)2 anti-human IgG-Fc, PE conjugated, preabsorbed, Abcam, cat #98596) is incubated with the antibody-bound microspheres. After additional washing steps the Multiplex ELISA plates are measured and analyzed using the Luminex® 200™ System and the Luminex Xponent 3.1 software, respectively.

### Neutralization Test

EBV-specific neutralizing antibodies in NHP serum samples are quantified by a flow cytometry-based neutralization test. Briefly, 2-fold serial dilutions of test sera are prepared and a defined amount of EBV is added to each serum dilution. After 1 hour, the serum-virus mixes are added to Ramos cells. Infected Ramos cells express EBV proteins. The next day, the cells are stained with DAPI and a monoclonal EBV-specific antibody. The percentage of EBV-positive cells is analyzed using an LSR Fortessa flow cytometer. Serum samples containing neutralizing antibodies lead to a reduced percentage of EBV-positive cells. Conversely, serum samples which do not contain neutralizing antibodies show the highest percentage of anti-EBV stained cells. Non-infected “Cells only” wells are used to set the gate for EBV-positivity. The percentage of EBV-positive cells is used to calculate the IC50 titer using the GraphPad Prism software.

### Enzyme-Linked Immunosorbent Assay (ELISA)

Serum OVA-specific IgGs were determined. ELISAs were performed by coating 96-well plates with 5 μg/ml OVA, followed by blocking with PBS containing 5% FCS/0.05% Tween20. IgG was detected using HRP-conjugated antibodies, followed by TMB substrate. Absorbance was measured at 450 nm. ELISA titers were determined using linear regression analysis and Log 10 titers calculated.

### Example 2: Experimental Design

The objective of this analysis was to evaluate and compare in serum EBV-specific IgG and neutralizing antibodies induced by the different prime-boost vaccination regimens using MVA-BN-EBV, VRP-BN011 and AdC68gp350 administered IM two times, four weeks apart to cynomolgus monkeys (Macaques fascicularis).

Twelve female cynomolgus monkeys were administered three different vaccines via intramuscular injection (in the right thigh) on Days 1 and 29 of the dosing phase. The terminal necropsy took place after 6 weeks.

The main study design for the respective vaccine administrations is summarized in Table 1. To determine the immunogenicity in terms of antibodies, EBV-specific IgG for EBV antigen gp350 as well as EBV neutralizing antibodies were determined using Multiplex ELISA and a flowcytometry based assay, respectively. Non-human primate (NHP) serum samples from pre-dose, before and two weeks after second administration (Day 29, Day 43) were analyzed.

TABLE 1
Main Study Design and Group Assigment.

| | Test Item | | | | | | | | Necropsy |
|---|---|---|---|---|---|---|---|---|---|
| Group | Dose/1^{st} | Dose | | Dose/2^{nd} | Dose | | Route of | Female | after |
| number | administration day 1 | volume | | administration day 29 | volume | | administration | numbers | 6 weeks |
| 1 | MVA-BN-EBV | 0.5 | mL | MVA-BN-EBV | 0.5 | mL | IM | 3 | 3 |
| | (5 × 10⁸ TCID₅₀) | | | (5 × 10⁸ TCID₅₀) | | | | | |
| 2 | VRP-BN011 | 2 | mL | MVA-BN-EBV | 0.5 | mL | IM | 3 | 3 |
| | (6.86 × 10⁹ TU) | | | (5 × 10⁸ TCID₅₀) | | | | | |
| 3 | VRP-BN011 | 2 | mL | VRP-BN011 | 2 | mL | IM | 3 | 3 |
| | (6.86 × 10⁹ TU) | | | (6.86 × 10⁹ TU) | | | | | |
| 4 | VRP-BN011 | 2 | mL | AdC68gp350 | 0.5 | mL | IM | 3 | 3 |
| | (6.86 × 10⁹ TU) | | | (1 × 10¹¹ vp) | | | | | |

### Example 3: Results and Conclusion

In subsequent sections, the following abbreviations were used:

| | |
|---|---|
| AdC68gp350 | Adenovirus vector (AdC68) expresses a truncated soluble form |
| | of the EBV gp350 protein with a flexible linker and a |
| | multimerization domain for multimer formation |
| EBV | Epstein-Barr Virus |
| ELISA | Enzyme linked immunosorbent assay |
| GCN4 | Yeast protein GCN4 derived leucine zipper based |
| | multimerization domain |
| gH | Glycoprotein H of EBV |
| gH/gL/gp42 complex | EBV gH/gL/gp42 complex |
| gL | Glycoprotein L of EBV |
| gp350 | EBV glycoprotein 350 (here a soluble version comprising amino |
| | acids 2-434 was used) |
| Inf.U | Infectious units |
| IgG | Immunoglobulin G |
| IC50 | Half maximal inhibitory concentration |
| IM | Intramuscular |
| LLOQ | Lower limit of quantification |
| MVA-BN | Modified vaccinia Ankara - Bavarian Nordic |
| MVA-BN-EBV | MVA-BN vector encoding the non-structural proteins EBNA3A |
| | and a fusion of the early transactivators BRLF1/BZLF1 as well |
| | as the structural glycoproteins (gp) gH and gL and a truncated |
| | soluble form of gp350 (amino acids 2-434) with a flexible linker |
| | and a multimerization domain for multimer formation |
| NHP | Non-human primate |
| SOP | Standard operating procedure |
| VRP-BN011 | Recombinant VRP-BN containing a VEEV-TC83 derived |
| | replicon encoding EBV soluble gp350 (amino acids 2-434) with |
| | a flexible linker and a GCN4 multimerization domain for |
| | multimer formation, gH and gL packaged with VEEV-TC83 |
| | derived envelope and capsid proteins |
| TU | Transduction units |

### Example 4: Gp350-Specific IgG Antibody Response in NHP

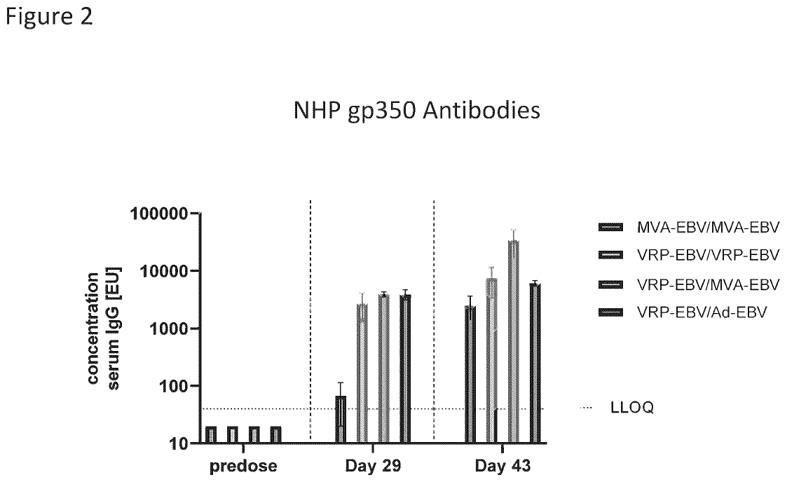

EBV gp350-specific group geometric mean concentration (GM) are depicted in Table 2 and in FIG. 2. The lower limit of quantification (LLOQ) was defined to be 40 EU. Serum samples below 40 EU were reported negative as 20 EU, which corresponds to half of the LLOQ.

Predose serum samples of all groups (Group 1 to 4) were negative.

At Day 29 (4 weeks after first administration), complete seroconversion determined by multiplex ELISA was detected for NHPs of nearly all groups. Of Group 1 (MVA/MVA) just one out of three animals developed measurable gp350-specific IgG. Highest gp350-specific IgG concentrations with a GM of 3563, 3891 and 3731 were measured for Group 2 (VRP/MVA) and Group 4 (VRP/Ad), respectively at Day 29.

At Day 43 (2 weeks post second administration) complete seroconversion was also observed for Group 1 (MVA/MVA). The second administration boosted the gp350-specific antibody response 4-fold in Group 3 (VRP/VRP) and 7-fold in group 2 (VRP/MVA) compared to the respective antibody concentrations at Day 29 . . . . Group 2 had the highest gp350-specific IgG response with a GM of 26.626 (individual concentrations ranging from 14.406 to 71453), followed by group 3 (VRP/VRP) and 4 (VRP/Ad) with a GM of 4800 (individual concentrations ranging from 1180 to 15.032) and a GM of 6090 (individual concentrations ranging from 5179 to 7225), respectively.

These results indicate that VRP strongly increased gp350-specific antibody responses in heterologous combinations with Ad or MVA as boost vaccination and that homologous combinations of MVA or VRP had the least immunogenic effect in terms of gp350-specific antibody induction.

TABLE 2
Summary of gp350-sepcefic IgG antibody concentrations
determined by multiplex ELISA

| | Group 1 | | | Group 2 | | | Group 3 | | | Group 4 | | |
|---|---|---|---|---|---|---|---|---|---|---|---|---|
| | MVA/MVA | | | VRP/MVA | | | VRP/VRP | | | VRP/Ad | | |
| Time | %¹ | GM² | N³ | %¹ | GM² | N³ | %¹ | GM² | N³ | %¹ | GM² | N³ |
| predose | 0 | 20 | 3 | 0 | 20 | 3 | 0 | 20 | 3 | 0 | 20 | 3 |
| Day 29 | 100 | 40 | 3 | 100 | 3891 | 3 | 100 | 1110 | 3 | 100 | 3731 | 3 |
| Day 43 | 100 | 2072 | 3 | 100 | 26626 | 3 | 100 | 4800 | 3 | 100 | 6090 | 3 |

¹% Seroconversion rate,
²Geometric mean concentration,
³Number of NHP

### Example 5: EBV-Specific Neutralizing Antibody Response in NHP

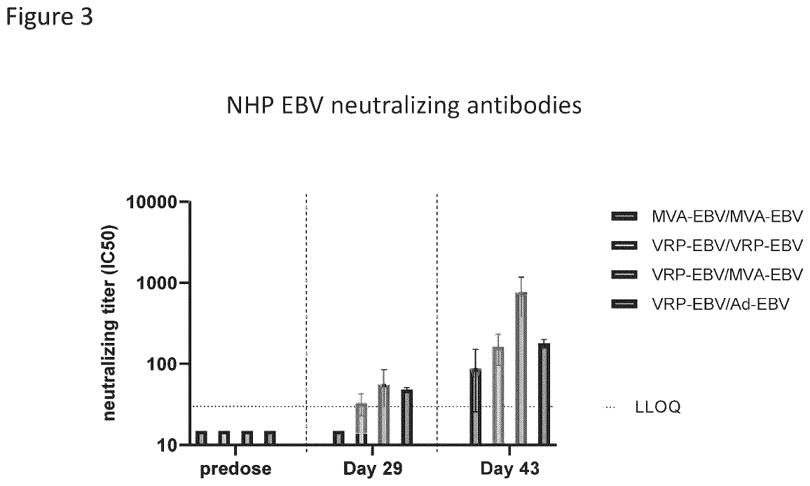

EBV-specific neutralizing antibody responses were measured in all predose, Day 29 and Day 43 serum samples for each group by a flowcytometry-based neutralizing test. Neutralizing group geometric mean concentrations (GM) are depicted in table 3 and in FIG. 3. The LLOQ was defined to be the half maximal inhibitory concentration (IC50) of 30. Serum samples below an IC50 of 30 were reported negative as IC50 of 15, which corresponds to half of the LLOQ.

Predose serum samples of all groups (Group 1 to 4) were negative.

At Day 29 (4 weeks after first administration) seroconversion was measured for two out of three animals in Group 2 (VRP/MVA) and Group 3 (VRP/VRP) and 100% sero-conversion was detected in Group 4 (VRP/Ad). Animals of Group 1 (MVA/MVA) did not seroconvert at this time point. In general, the neutralizing antibody concentration for all seroconverted animals independent of the respective prime vaccination was low (GM<50) at Day 29.

At Day 43 (2 weeks post second administration) complete seroconversion was observed for all groups but Group 1 (MVA/MVA) with just two out of three animals having developed measurable neutralizing antibodies.

The second administration boosted the neutralizing antibody levels 14-fold in Group 2 (VRP/MVA). The least boost effect was noticed for Group 1 (MVA/MVA; 3-fold), Group 3 (VRP/VRP; 4-fold) and Group 4 (VRP/Ad; 4-fold). The highest neutralizing concentration with a GM of 605 (individual concentrations ranging from 372 to 1572) was measured for group 2 (VRP/MVA). Group 4 (VRP/Ad), Group 3 (VRP/VRP) and Group 1 (MVA/MVA) had the lowest neutralizing antibody levels with a GM of 176 (individual concentrations ranging from 145 to 219), a GM of 118 (individual concentrations ranging from 31 to 256) and a GM of 49 (individual concentrations ranging from 15 to 214), respectively.

These results correlate to the gp350-specific IgG responses measured by multiplex ELISA: VRP strongly boosted neutralizing antibody responses in heterologous prime-boost combinations. Homologous prime-boost regimes with MVA or VRP were least immunogenic.

TABLE 3
Summary of neutralizing antibodies

| | Group 1 | | | Group 2 | | | Group 3 | | | Group 4 | | |
|---|---|---|---|---|---|---|---|---|---|---|---|---|
| | MVA/MVA | | | VRP/MVA | | | VRP/VRP | | | VRP/Ad | | |
| Time | %¹ | GM² | N³ | %¹ | GM² | N³ | %¹ | GM² | N³ | %¹ | GM² | N³ |
| predose | 0 | 15 | 3 | 0 | 15 | 3 | 0 | 15 | 3 | 0 | 15 | 0 |
| Day 29 | 0 | 15 | 3 | 67 | 42 | 3 | 67 | 29 | 3 | 100 | 48 | 100 |
| Day 43 | 67 | 49 | 3 | 100 | 605 | 3 | 100 | 118 | 3 | 100 | 176 | 100 |

¹% Seroconversion rate,
²Geometric mean concentration,
³Number of NHP

In summary, the heterologous prime-boost regimens with VRP as prime vaccination and MVA as booster vaccination were highly immunogenic in terms of gp350-specific lgG and neutralizing antibodies while the homologous vaccination regimens with MVA or VRP had the least immunogenic effect. A single vaccination independent of the vaccine candidate was not sufficient to induce a strong neutralizing antibody response.

### Example 6: EBV-Specific T Cell Response in NHP

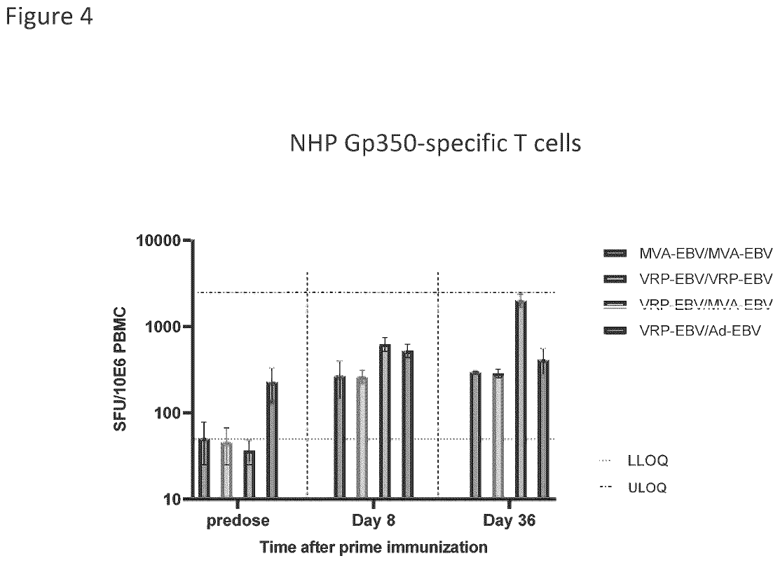

EBV-specific T cell responses were measured in all predose, Day 29 and Day 43 blood PBMC samples for each group by ELISPOT. Spot forming units (SFU) per 1×10⁶ PBMCs are depicted in FIG. 4.

Predose serum samples of groups (Group 1 to 3) were below LLOQ exept for group 4.

At Day 8 after first administration all 4 groups showed detectable Gp350-specific T cell responses with no major differences between the groups. The second vaccination did not further enhance Gp350-specific T cell responses in Group 1 (MVA/MVA), Group 3 (VRP/VRP) and Group 4 (VRP/Ad) with the exception of Group 2 (VRP/MVA) that showed a further increase of SFU per 1×10⁶ PBMCs (FIG. 4). This data demonstrates that heterologous immunization of VRP/MVA not only excelled in antibody induction but also resulted in the highest EBV-specific T cell responses.

### Example 7: EBV-Specific T Cell Response in Mice

To assess T cell immunogenicity, mice were prime/boost immunized on day 0 and day 21 using MVA-EBV or VRP-EBV in homologous or heterologous combination (see Table 4). The experiment was terminated 2 weeks after the boost.

TABLE 4
Design of VRP-EBV/MVA-EBV prime/boost study in mice

| Prime (Day 0) | Boost (Day 21) |
|---|---|
| PBS | PBS |
| MVA-EBV | MVA-EBV |
| VRP-EBV | MVA-EBV |
| VRP-EBV | VRP-EBV |

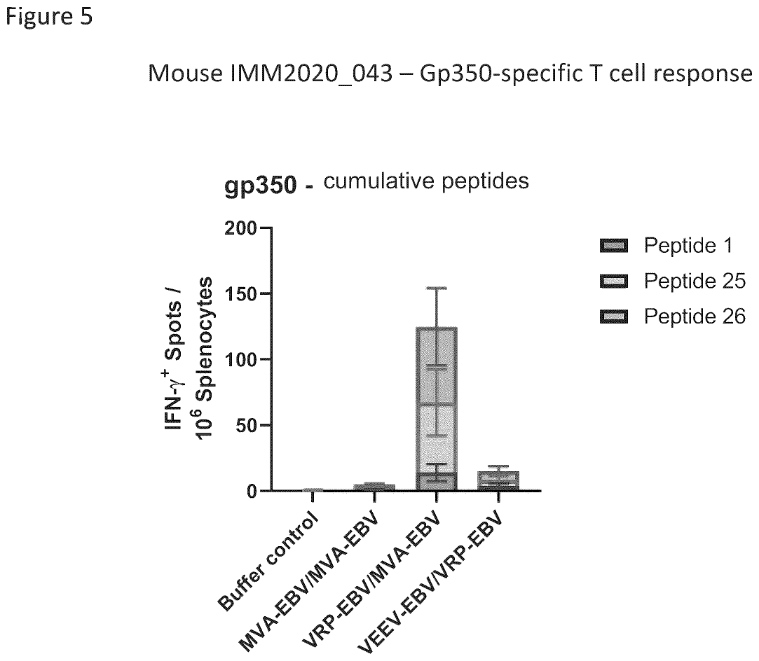

Prior to this experiment the immunodominant peptides within Gp350 have been identified using a peptide library screening approach. With that 3 peptides (referred as peptide 1, peptide 25 and peptide 26) have been identified to be efficiently recognized by T cells after immunization (data not shown). Accordingly, these three peptides were used for ELISPOT and 6h re-stimulation assays in the experiments reported in the following. FIG. 5 shows that homologous immunization of MVA/MVA or VRP/VRP did hardly induce T cell responses to EBV. In contrast to this, heterologous immunization with VRP followed by MVA drastically increased the number of SFU/1×10⁶ splenocytes indicative of strong EBV-specific T cell induction by this immunization regimen. Of note, peptides 25 and 26 were dominant over peptide 1.

### Example 8: OVA-Specific T Cell Response in Mice

In order to investigate whether the advantage of heterologous combination of the VRP and MVA platform also extends to other antigens but EBV, another experiment was set up. In this experiment the model antigen Ovalbumin (OVA) was selected and accordingly MVA-OVA and VRP-OVA has been used for immunization (see Table 5). To assess T cell immunogenicity, mice were prime/boost immunized on day 0 and day 21 using MVA-OVA or VRP-OVA in homologous or heterologous combination (see Table 5). The experiment was terminated 2 weeks after the boost.

TABLE 5
Design of VRP-OVA/MVA-OVA prime/boost study in mice

| Prime (Day 0) | Boost (Day 21) |
|---|---|
| PBS | PBS |
| MVA-OVA | MVA-OVA |
| VRP-OVA | VRP-OVA |
| VRP-OVA | MVA-OVA |

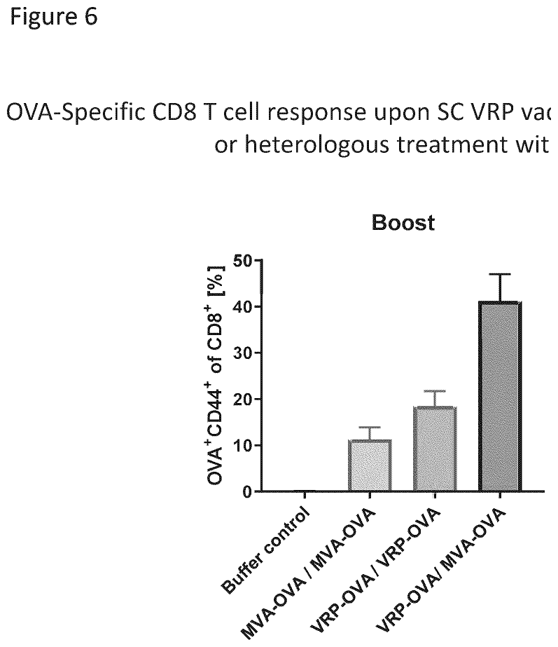

Main read-outs were CD8 T cell responses in peripheral blood five days after boost immunizations, and peptide restimulation of splenocytes against dominant and subdominant epitopes of OVA were performed at the day of sacrifice of the mice (4 weeks after boost). Five days after the boost, when groups had split up in homologous and heterologous treatments, the heterologous treatments showed the strongest responses (FIG. 6). At this time point, the homologous treatment with VRP-OVA showed a tendency to higher responses than the homologous MVA-OVA treatment (MVA/MVA), yet this was not statistically significant. In contrast, boosting an initial VRP-OVA immunization with an MVA-OVA immunization led to a significantly stronger expansion of OVA-specific CD8 T cells 5 days after the boost in the blood compared to the MVA/MVA treatment and compared to the VRP/VRP treatment (FIG. 6).

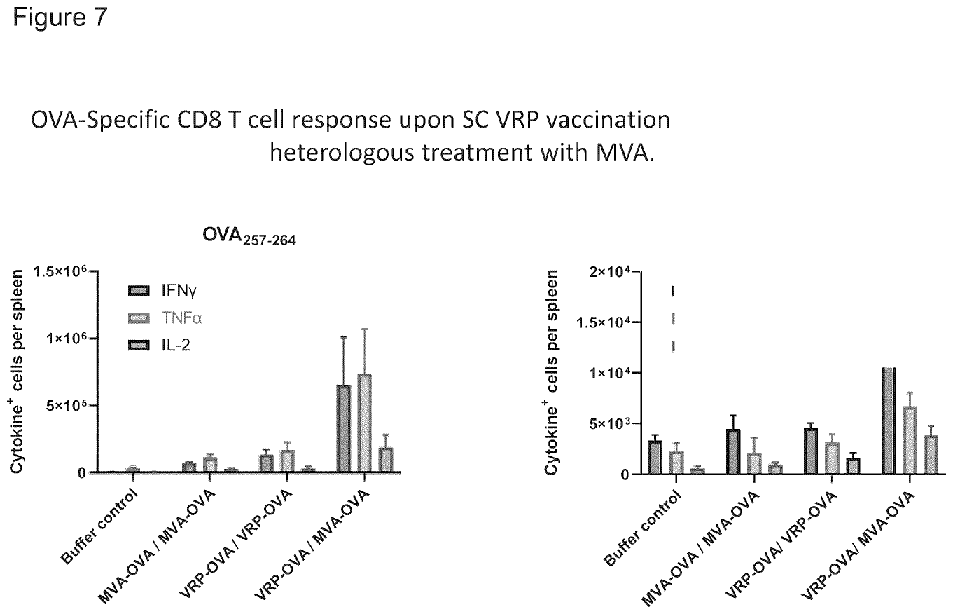

Four weeks after the boost, early memory responses were tested in splenocytes. For this, splenocytes were re-stimulated with the OVA257-264 SIINFEKL peptide as well as the OVA55-62 subdominant peptide. As shown in FIG. 7, the combination of a VRP-OVA prime immunization with an MVA-OVA boost immunization led to the strongest responses for both, the OVA257-264 peptide as well as the OVA55-62 peptide.

Interestingly, while all treatment combinations could induce an OVA257-264 peptide-specific response on their own, the response to the subdominant OVA55-62 peptide was only detectable in the heterologous combination group. This indicates, that combining a VRP-OVA prime immunization with an MVA-OVA boost immunization is resulting not only in a stronger, but also broader immune response, that can hardly be achieved by homologous treatments on their own.

### Example 9: OVA-Specific CD8 T Cell Response in Mice

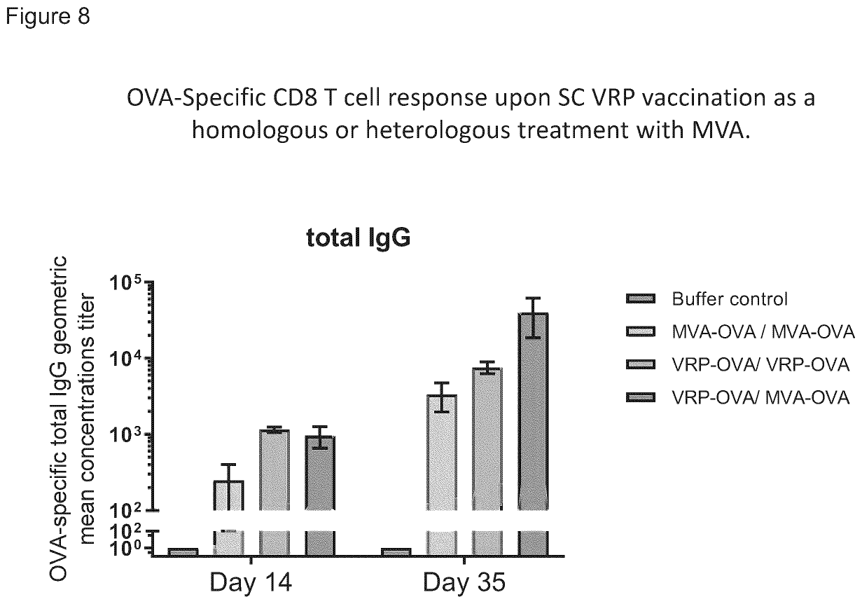

Furthermore, OVA-specific antibody production induced by homologous or heterologous combination of the VRP-OVA and MVA-OVA platform was tested. To assess antibody immunogenicity, mice were prime/boost immunized on day 0 and day 21 using MVA-OVA or VRP-OVA in homologous or heterologous combination (see Table 5). Antibody production was measured in the serum at days 14 and 35, 14 days after prime and boost immunizations, respectively. As shown in FIG. 8, the highest total IgG titers and complete seroconversion at day 14 were achieved by immunization with VRP-OVA whereas MVA-OVA immunized mice showed slightly weaker antibody responses. After the boost immunizations (day 35), OVA-specific antibody levels in all treatment groups were increased and showed complete seroconversion, however, there was a clear benefit of boosting VRP-OVA prime immunization with an MVA-OVA immunization.

In summary, the combination of a VRP-OVA prime immunization with an MVA-OVA boost immunization enhances antigen-specific CD8+ T cell expansion in peripheral blood, as well as increases antigen-specific CD8+ T cell qualitative responses towards both dominant and subdominant OVA epitopes. The benefits of heterologous VRP/MVA immunization were also observed in terms of antibody induction.

### Example 10: Gp350-Specific T Cell Response in Mice

So far, the above experiments were performed with VRPs that are based on Venezuelan Equine Encephalitis Virus (VEEV). However, VRPs can also be based on other alphaviruses. Accordingly, a Semliki Forest Virus (SFV) based VRP was generated to investigate if this type of VRP can also potently induce immune responses, or if it is even more immunogenic than the VEEV-based VRPs. To this end the immunogenicity of SFV-based VRPs expressing the EBV antigens gp350, gH and gL (SFV-VRP-EBV) was tested.

To assess T cell immunogenicity, mice were prime/boost immunized on day 0 and day 21 using MVA-EBV or SFV-VRP-EBV in homologous or heterologous combination (see Table 6). The experiment was terminated 2 weeks after the boost.

TABLE 6
Design of SFV-VRP-EBV/MVA-EBV prime/boost study in mice

| Prime (Day 0) | Boost (Day 21) |
|---|---|
| PBS | PBS |
| MVA-EBV | MVA-EBV |
| SFV-VRP-EBV | MVA-EBV |
| SFV-VRP-EBV | SFV-VRP-EBV |

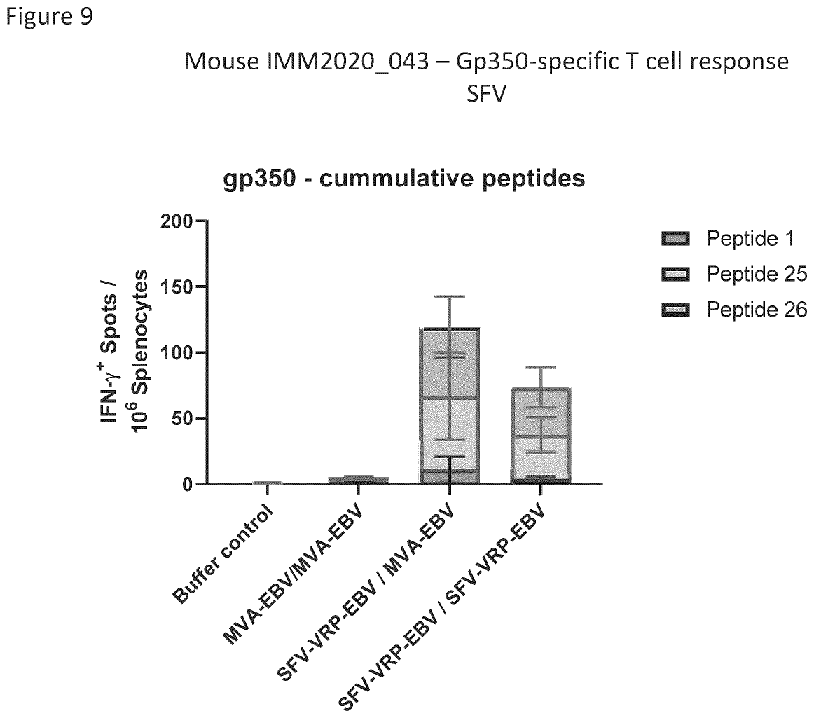

Prior to this experiment the immunodominant peptides within Gp350 have been identified using a peptide library screening approach. With that, 3 peptides (referred as peptide 1, peptide 25 and peptide 26) have been identified to be efficiently recognized by T cells after immunization (data not shown). Accordingly, these three peptides were used for ELISPOT and 6h re-stimulation assays in the experiments reported in the following. FIG. 9 shows that homologous immunization of MVA/MVA did hardly induce T cell responses to EBV. In contrast to this, SFV-VRP elicited height EBV-specific T cell responses to EBV (FIG. 9). Importantly, heterologous immunization with SFV-VRP followed by MVA drastically increased the number of SFU/1×10⁶ splenocytes indicative of strong EBV-specific T cell induction by this immunization regimen. Of note, peptides 25 and 26 were dominant over peptide 1.

In conclusion, heterologous immunization of VRPs that are based on Venezuelan Equine Encephalitis Virus (VEEV) or Semliki Forest Virus (SFV) is a potent inducer of T cells specific to vaccine encoded antigens.

SEQUENCES
Nucleic acid sequence of gp350 multimer (1455 nucleotides).
SEQ ID NO: 1
atggaagcagctctgctcgtgtgccagtacaccatccagagcctgatccacctgacaggagagg
atcctggcttcttcaacgtggaaatcccagagtttcccttctaccctacctgcaacgtgtgcac
agccgacgtgaacgtgaccatcaacttcgacgttggaggcaagaagcaccagctggacctggat
ttcggacagctgacacctcacaccaaggctgtgtatcagcctagaggagcctttggtggcagcg
agaacgccaccaacctgtttctgctggaactgcttggagctggcgagctcgcactgaccatgag
aagcaagaaactgcccatcaatgtgaccacaggcgaggaacagcaggtgtccctggaaagcgtg
gacgtgtactttcaagacgtgttcggcaccatgtggtgccaccacgccgagatgcagaaccctg
tgtacctgatcccagagacagtgccctacatcaagtgggacaactgcaacagcaccaacatcac
agccgtcgtgagagctcagggactggatgtgacactgcctctgagcctgcctaccagtgcccag
gacagcaacttcagcgtgaagaccgagatgctgggcaacgagatcgacatcgagtgcatcatgg
aagatggcgagatcagccaggtgctgcctggcgacaacaagttcaacatcacatgcagtggcta
cgagagccacgtgccatctggaggcatcctgaccagcacaagcccagtggccacacccatccct
ggcacaggctacgcctacagcctgagactgacacccagacccgtgtccagattcctgggcaaca
acagcatcctgtacgtgttctactctggcaacggacccaaggcctctggtggcgattactgtat
ccagagcaacatcgtgttcagcgacgagatccctgccagccaggacatgccaaccaataccacc
gacatcacctacgtgggagacaatgccacctacagcgtgcccatggtcacctccgaggacgcca
acagccctaatgtgaccgtgacagccttctgggcatggcctaacaacaccgagacagacttcaa
gtgcaagtggaccctgacctctggcacacctagtggctgcgagaatatcagcggagccttcgcc
agcaaccggaccttcgatatcaccgtgtctggccttggcacagctcccaagaccctgatcatca
ccaggactgccaccaatgccacaaccacaacccacaaagtgatcttcagcaaggctcctgagag
caccacaactagtcctacactgcctaagcccagcacacctcctggcagctcttgtggaggcatg
aaagtgaagcagctggtggacaaggtggaagaactgctgagcaagaactaccacctcgtgaatg
aggtggcacggctcgtgaagctcgtgggagaaagaggtggctgatag
Nucleic acid sequence of gH (2121 nucleotides).
SEQ ID NO: 2
atgcagctgctgtgcgtgttctgcctggtgctgctgtgggaagtgggagctgccagcctgagcg
aagtgaagctgcacctggacatcgagggccacgccagccactacaccatcccttggacagagct
gatggccaaggtgcctggactgtctcctgaagctctgtggcgagaagccaacgtgaccgaggat
ctggcttccatgctgaaccggtacaagctgatctacaagaccagcggaaccctgggaatcgctc
tggcagagcctgtggatatccctgctgtgtctgagggcagcatgcaggtggacgccagcaaagt
gcacccaggagtgatcagcggactgaatagtcctgcctgtatgctgagcgctcctctggaaaag
cagctgttctactacatcggcaccatgctgcctaacaccagaccccacagctacgtgttctacc
agctgcggtgccacctgagctacgtcgctctgagcatcaacggagacaagttccagtacaccgg
agctatgaccagcaagttcctgatgggtacctacaagagagtgaccgagaagggagacgaacac
gtgctgagcctggtgttcggcaagaccaaggacctgcctgacctgagaggacccttcagctacc
ctagcctgacaagcgctcagagcggagactacagcctcgtgatcgtgaccaccttcgtgcacta
cgccaacttccacaactacttcgtgcccaacctgaaggacatgttcagcagagccgtgaccatg
acagctgccagctacgccagatacgtgctgcagaaactggtgctgctggaaatgaagggaggat
gcagagagcctgagctggacaccgagacactgacaaccatgttcgaggtgtccgtggccttctt
caaagtgggacacgctgtgggagagacaggcaatggctgtgtggacctgagatggctggccaag
agcttcttcgagctgaccgtgctgaaggatatcattggcatctgctacggagccaccgtgaagg
gcatgcagagctacggactggaaagactggcagctatgctgatggctaccgtgaagatggaaga
actgggacacctcaccacagagaagcaggaatacgctctgagactggccaccgtgggctatcct
aaagctggagtgtactccggactgatcggtggagctacaagcgtgctgctgagtgcctacaacc
gacaccctctgttccagcctctgcacaccgtgatgagagagacactgttcatcggaagccacgt
ggtgctgcgagagctgagactgaatgtgaccacacagggacctaacctggctctgtatcagctg
ctgagcaccgctctgtgtagcgctctggagatcggagaggtgctgagaggactggctctgggca
cagagagcggactgttcagcccttgctacctgagcctgagattcgacctgaccagagacaagct
gctgtccatggctcctcaggaagccacactggatcaggcagccgtgtccaacgctgtggatggc
tttctgggacgactgtcactggaaagagaggacagggacgcctggcatctgcctgcctataagt
gcgtggaccgactggacaaggtgctgatgatcattcccctgatcaatgtgaccttcatcatcag
ctccgaccgagaggtgcgaggcagtgccctgtatgaagccagcaccacatacctgagcagcagc
ctgttcctgagccctgtgatcatgaacaagtgcagccagggagctgtggctggagagcctagac
agatccccaagatccagaacttcacccgaacccagaagtcctgcatcttctgtggcttcgctct
gctgtcctacgacgagaaagagggactggaaaccaccacctacatcaccagccaggaagtgcag
aacagcatcctgtccagcaattacttcgacttcgacaacctgcatgtgcactacctgctgctga
ccacaaacggcacagtgatggaaatcgctggactgtacgaggaacgagctcatgtggtgctggc
catcatcctgtactttatcgcctttgctcttgggatcttcctggtgcacaagatcgtgatgttc
ttcctgtga
Nucleic acid sequence of gL (414 nucleotides).
SEQ ID NO: 3
atgagagccgtgggagtgttcctggccatctgcctcgtgaccatcttcgtgctgcccacctggg
gtaactgggcttacccttgttgccacgtgacccagctgagagcccagcatctgctggcactgga
gaacatcagcgacatctacctggtgtccaaccagacctgcgacggcttcagcctggcatccctg
aacagtcccaagaacggcagcaatcagctcgtgatctccagatgtgccaacggactgaacgtgg
tgtccttcttcatctccatcctgaagcggagcagcagcgctctgacaggccacctgagagagct
gctgaccaccctggaaaccctgtacggcagcttcagcgtggaagatctgttcggagccaacctg
aacagatacgcctggcacagaggaggctga
Nucleic acid sequence of BZLF1-BRLF1 (2283 nucleotides).
SEQ ID NO: 4
atgagcctggtgtccgactactgcaacgtgctgaacaaagagttcacagctggcagcgtggaaa
tcactctgcggagctacaagatctgcaaggccttcatcaacgaggccaaggctcatggcagaga
atggggtggactgatggccaccctgaacatctgcaatttctgggctatcctgcggaacaacaga
gtgagacggagagccgagaacgctggcaatgatgcctgctctatcgcctgtcctatcgtgatga
gatacgtgctggaccacctgatcgtcgtgaccgaccggttcttcatccaagctcccagcaatag
agtgatgattcctgccaccatcggcacagccatgtacaagctgctgaagcacagtagagtgaga
gcctacacctacagcaaggtgctgggagtggacagagcagccatcatggctagtggcaaacagg
tggtggaacacctgaaccggatggagaaagagggactgctgagcagcaagttcaaggccttctg
caagtgggtgttcacctaccctgtgctggaagagatgttccagaccatggtgtccagcaagaca
ggacacctgaccgacgacgtgaaagatgtgagagctctgatcaagacactgcccagagccagct
acagctctcacgcaggtcagagaagctacgtgtcaggcgtgctgcctgcatgtctgctgtccac
caagagcaaggctgtggaaacacccatcctggtgtctggagccgacagaatggacgaagaactg
atgggcaacgacggtggagccagccatacagaggccagatactctgagtctggccagttccacg
ccttcaccgacgagctggaaagcctgcctagccctaccatgcctctgaaacctggagcccagtc
tgccgactgtggcgatagctcctcttcaagcagtgacagtggcaacagcgataccgagcagagc
gagagagaagaggctagagccgaagctcctagactgagagcacccaagagcagaagaaccagca
gacccaacagaggacagacaccctgtccttctaacgctgcagagcctgagcagccttggattgc
tgccgtgcaccaggaaagcgacgagagacctatcttcccacatcccagcaagccaaccttcctg
atgttcgatcctgctcctgaggcaggctctgccatctccgatgtgttcgagggacgggaagtgt
gccagcccaagcggatcagacccttccatcctcctggaagcccttgggctaacagacctctgcc
agcctctcttgctccaacacctacaggacctgtgcacgagcctgtgggcagcctgacaccagct
ccagtgcctcagcctctggatccagctcctgccgtgacacctgaggccagccatctgctggaag
atcccgacgaagagacaagccaggcagtgaaggccctgagagagatggctgatacagtgatccc
acagaaagaagaggcagccatttgtggccagatggacctgtctcaccctccacctagaggccac
ctggatgagctgaccacaaccctggaatccatgaccgaggacctgaacctggacagccctctga
ctcccgagctgaacgagatcctggacacctttctgaacgacgagtgcctgctgcacgccatgca
catcagcaccggagacagcatcttcgacaccagcctgttcatgatggaccctaacagcaccagc
gaggacgtgaagttcactcccgacccttaccaggtgcccttcgtgcaggccttcgatcaggcca
ccgagaatgcctgcagaagtgcctacaagcaggacgaccagcactacagagagcctgagcctct
gcctcagggacagctgacagcctaccacgtgtcacagcctgcacccgagaacgcctaccaggcc
tatgctgcacctcagctgtttcccgtgtccgacatcacccagaaccaacagaccaaccaggctg
gaggcgaagctcctcagcctggcgataatagcaccgtgcagacagctgcagctgtggtgtttgc
ttgccctggagctaatcagggtcagcagctggcagatattggcgtgccacagccagcacctgtg
gctgctcctgccagaaggaccagaaagcctcagcaacccgagagcctggaagagtgcgacagcg
aactggaaatcaagcggagcgagaacgacagactggaactgctgctgaaacagatgtgtcccag
cctggacgtggactccatcatccctagaacacccgactgataa
Nucleic acid sequence of EBNA3A (2892 nucleotides).
SEQ ID NO: 5
atggagacagacacactcctgctatgggtactgctgctctgggttccaggttccactggtgacg
acaaggacagacctggaccacctgccctggacgacaacatggaagaggaagttcccagcaccag
cgtggtgcaggaacaggtgtcagctggcgactgggagaacgtgctgatcgagctgagcgacagc
agcagcgagaaagaggctgaggacgcacatctggaacctgctcagaaaggcaccgtggaccatg
atgctggaggctctgctccagccagacctatgctgcctcctcagcctgatctgcctggcagaga
ggccatcctgagaagattcccactggacctgcggaccctgctgcaggctattggagcagctgcc
acacggatcgacaccagagccatcgaccagttcttcggcagccagatcagcaacaccgagatgt
acattatgtacgccatggccatcagacaggccattagggatcaggccaaatggaggctgcagac
actggctgcaggctggccgatgggttaccaggcctacagcagctggatgtacagctacaccgac
caccagaccacacccaccttcgtgcatctgcaagcagcagctggagctactggagggagaagat
gccacgtgacattcagtgctggcaccttcaagctgcccagatgcacacctggagacagacagtg
gctgtacgtgcagtctagcgtgggcaacatcgtgcagagctgcaaccctcggtacagcatcttc
ttcgactacatggccattcaccggtctctgaccaagatctgggaagaggtgctgacaccagacc
agagagtgtcctttatggaattcctgggcttcctgcagcggaccgacctgagctacatcaagag
cttcgtgtccgacgctctgggcaccaccagcatccagactccctggatcgacgacaaccctagc
acagaaacagctcaggcttggaacgcaggcttcctgagaggcagagcctacggcatcgacctgc
tgagaacagagggagagcatgtggaaggagctaccggtgaaaccagagaggaaagcgaggacac
cgagagcgacggagacgacagactgctgctgatgaccgagcaaggcaaagaagtgctggagaag
gccagaggctccacctacggcacacctagacctcctgtgcccaagcctagacctgaggtgccac
agagcgacgagacagccacatctcacggctctgcccaggtgcctgagccacctacaattcatct
ggcagctcagggcatggcctacccactgcatgaacagcacggcatggctccttgtcctgtggct
caggcacctcctacacctctgcctcctgtgtctcctggcgatcagctgcctggcgtgttcagcg
acggaagagtggcctgtgctcctgttcctgcacctgcaggaccaattgtgagaccttgggagcc
tagcctgacacaggctgcaggacaggcctttgctccggtgagacctcagcacatgcctgtggaa
cctgtgccagtgcctaccgttgccctggaaagacccgtgtaccctaagcctgtgaggccagctc
cacccaagattgccatgcagggacctggtgagacaagtggcatttggaggcctgctccttggac
acccaatccacctagaagcccttcgcagatgagcgtgctgagagccgaggcacaagtgaagcag
gccagcgtggaagtgcagccacctcagctgactcaggtgtcacctcagcagcccatggagggac
ctctggtacctgagcagcagatgtttcctggtgctcctttcagccaggtggctgatgtcgtgag
ggctcctggcgtgccagctatgcagccacagtacttcgacctgcctctgatccagcccatcagc
cagggagcaccagtggctcctctgagagcctctatgggacctgtgcctccagtgccagcaaccc
agcctcagtatttcgatatccctctgaccgagcctatcaatcagggagcctctgcagcacactt
cctgccacagcagcctatggagggaccactggtgcctgaacaatggatgttcccaggagctgct
ctgagccagtctgtgagaccaggcgtggcacagagccagtactttgatctgcctctgacacagc
caatcaaccacggagcacctgctgctcactttctgcaccaacctccaatggaaggtccttgggt
accagagcagtggatgtttcagggagctcctcctagccagggcaccgatgtggtgcagcatcag
ctggacgctctgggctacacactgcacggactgaatcatccaggtgtgccagtgtctccagccg
ttaatcagtaccacctgagccaggctgccttcggcctgcccattgatgaggatgagagcggaga
gggcagcgacacatctgagccttgcgagatccacggcagaccctgtcctcaggcaccagaatgg
ccagttcaggaagaaggaggccaggacgccaccgagattcacggaaggcctagacccagaactc
ctgagtggccagtgcagggagagggtggacagaatgtggctggtcctgagactagacgggtggt
ggtgtctgctgtggtgcacatgtgtcaggacgacgagttccctgacctgcaggatcctcctgat
gaggccggagggggtggctctggtgggggagggtccggcggaggcggttcagctgtgggccagg
acacgcaggaggtcatcgtggtgccacactccttgccctttaaggtggtggtgatctcagccat
cctggccctggtggtgctcaccatcatctcccttatcatcctcatcatgctttggcagaagaag
ccacgttgataa
Nucleic acid sequence of one loxPV site.
SEQ ID NO: 6
ATAACTTCGTATAGGATACTTTATACGAAGTTAT
Nucleic acid sequence of the Pr13.5 long promoter.
SEQ ID NO: 7
taaaaatagaaactataatcatataatagtgtaggttggtagtattgctcttgtgactagagac
tttagttaaggtactgtaaaaatagaaactataatcatataatagtgtaggttggtagta
Nucleic acid sequence of the PrS promoter.
SEQ ID NO: 8
aaaaattgaaattttattttttttttttggaatataa
Nucleic acid sequence of the PrH5m promoter.
SEQ ID NO: 9
taaaaattgaaaataaatacaaaggttcttgagggttgtgttaaattgaaagcgagaaataatc
ataaataatttcattatcgcgatatccgttaagtttgtatcgta
Nucleic acid sequence of Pr1328 promoter.
SEQ ID NO: 10
tatattattaagtgtggtgtttggtcgatgtaaaatttttgtcgataaaaattaaaaaataact
taatttattattgatctcgtgtgtacaaccgaaatc
Nucleic acid sequence of a 2A peptide (T2A).
SEQ ID NO: 11
AGCGGAGAGGGCAGAGGAAGTCTGCTAACATGCGGTGACGTCGAGGAGAATCCTGGACCT
Nucleic acid sequence of 2A peptide (P2A).
SEQ ID NO: 12
GGATCCGGCGCCACCAATTTCTCCCTGCTGAAACAGGCCGGCGATGTGGAAGAGAATCCAGGCC
CT
Nucleic acid sequence of flexible linker and GCN4
multimerization domain
SEQ ID NO: 13
CCTAAGCCCAGCACACCTCCTGGCAGCTCTTGTGGAGGCATGAAAGTGAAGCAGCTGGTGGACA
AGGTGGAAGAACTGCTGAGCAAGAACTACCACCTCGTGAATGAGGTGGCACGGCTCGTGAAGCT
CGTGGGAGAAAGAGGTGGC

We claim:

1. A vaccine combination comprising
(a) a first composition comprising an immunologically effective amount of a saRNA comprising a nucleic acid encoding an antigenic protein, together with a pharmaceutically acceptable carrier; and
(b) a second composition comprising an immunologically effective amount of an MVA vector comprising a nucleic acid encoding an antigenic protein, together with a pharmaceutically acceptable carrier;
wherein one of the compositions is a priming composition and the other composition is a boosting composition.

2. The vaccine combination according to claim 1, wherein the first composition is used for priming an immune response and the second composition is used for boosting said immune response.

3. The vaccine combination according to claim 1, wherein the second composition is used for priming an immune response and the first composition is used for boosting said immune response.

4. The vaccine combination according to claim 1, wherein the antigenic protein is an infectious disease antigen or a tumor-associated antigen.

5-7. (canceled)

8. The vaccine combination according to claim 1, wherein the antigenic proteins are any of the structural and non-structural proteins of EBV.

9. The vaccine combination according to claim 8, wherein the antigenic proteins are selected from gp350, gH, gL, EBNA3A, and BRLF1/BZLF1 fusion.

10. The vaccine combination according to claim 9, wherein the antigenic proteins are encoded by SEQ ID NO: 1, SEQ ID NO: 2, SEQ ID NO: 3, SEQ ID NO: 5 and SEQ ID NO: 4.

11. The vaccine combination according to claim 1, wherein the saRNA is a VRP, preferably based on an alphavirus, more preferably on VEEV, and, even more preferably based on TC83.

12. The vaccine combination according to claim 1, wherein the MVA is MVA-BN as deposited at the European Collection of Animal Cell cultures (ECACC) under accession number V00083008.

13. The vaccine combination according to claim 11, wherein the VRP in the first composition comprises a nucleic acid encoding an antigenic protein selected from the group consisting of gp350, gH and gL.

14. The vaccine combination according to claim 13, wherein the antigenic proteins are encoded by SEQ ID NO: 1, SEQ ID NO: 2, and SEQ ID NO: 3.

15-17. (canceled)

18. A kit comprising:
(a) a first composition comprising an immunologically effective amount of a saRNA comprising a nucleic acid encoding an antigenic protein, together with a pharmaceutically acceptable carrier; and
(b) a second composition comprising an immunologically effective amount of a MVA vector comprising a nucleic acid encoding an antigenic protein, together with a pharmaceutically acceptable carrier;
wherein one of the compositions is a priming composition and the other composition is a boosting composition.

19-38. (canceled)

39. A method of inducing an immune response against a virus in a subject, the method comprising administering to the subject:
(a) a first composition comprising an immunologically effective amount of a saRNA comprising a nucleic acid encoding antigenic proteins, together with a pharmaceutically acceptable carrier; and
(b) a second composition comprising an immunologically effective amount of a MVA vector comprising a nucleic acid encoding an antigenic protein, together with a pharmaceutically acceptable carrier;
wherein one of the compositions is a priming composition and the other composition is a boosting composition.

40. The method according to claim 39, wherein the first composition is used for priming an immune response and the second composition is used for boosting said immune response.

41. The method according to claim 39, wherein the second composition is used for priming an immune response and the first composition is used for boosting said immune response.

42-45. (canceled)

46. The method according to claim 39, wherein the antigenic protein is any of the structural and non-structural of EBV.

47. The method according to claim 46, wherein the antigenic proteins are selected from gp350, gH, gL, EBNA3A, and BRLF1/BZLF1 fusion.

48. The method according to claim 47, wherein the antigenic proteins are encoded by SEQ ID NO: 1, SEQ ID NO: 2, SEQ ID NO: 3, SEQ ID NO: 5 and SEQ ID NO: 4.

49. The method according to claim 39, wherein saRNA is a VRP based on VEEV.

50. (canceled)

51. The method according to claim 49, wherein the VRP in the first composition comprises a nucleic acid encoding an antigenic protein selected from the group consisting of gp350, gH and gL.

52. The method according to claim 51, wherein the antigenic proteins are encoded by SEQ ID NO: 1, SEQ ID NO: 2, and SEQ ID NO: 3.

53-54. (canceled)


SEQUENCE LISTING (19 sequences)

SEQ ID NO: 1 - DNA, 1455 bp, Human gammaherpesvirus 4
  source 1..1455: mol_type = genomic DNA
atggaagcagctctgctcgtgtgccagtacaccatccagagcctgatccacctgacaggagaggatcctggcttcttcaacgtggaaatcccagagtttcccttctaccctacctgcaacgtgtgcacagccgacgtgaacgtgaccatcaacttcgacgttggaggcaagaagcaccagctggacctggatttcggacagctgacacctcacaccaaggctgtgtatcagcctagaggagcctttggtggcagcgagaacgccaccaacctgtttctgctggaactgcttggagctggcgagctcgcactgaccatgagaagcaagaaactgcccatcaatgtgaccacaggcgaggaacagcaggtgtccctggaaagcgtggacgtgtactttcaagacgtgttcggcaccatgtggtgccaccacgccgagatgcagaaccctgtgtacctgatcccagagacagtgccctacatcaagtgggacaactgcaacagcaccaacatcacagccgtcgtgagagctcagggactggatgtgacactgcctctgagcctgcctaccagtgcccaggacagcaacttcagcgtgaagaccgagatgctgggcaacgagatcgacatcgagtgcatcatggaagatggcgagatcagccaggtgctgcctggcgacaacaagttcaacatcacatgcagtggctacgagagccacgtgccatctggaggcatcctgaccagcacaagcccagtggccacacccatccctggcacaggctacgcctacagcctgagactgacacccagacccgtgtccagattcctgggcaacaacagcatcctgtacgtgttctactctggcaacggacccaaggcctctggtggcgattactgtatccagagcaacatcgtgttcagcgacgagatccctgccagccaggacatgccaaccaataccaccgacatcacctacgtgggagacaatgccacctacagcgtgcccatggtcacctccgaggacgccaacagccctaatgtgaccgtgacagccttctgggcatggcctaacaacaccgagacagacttcaagtgcaagtggaccctgacctctggcacacctagtggctgcgagaatatcagcggagccttcgccagcaaccggaccttcgatatcaccgtgtctggccttggcacagctcccaagaccctgatcatcaccaggactgccaccaatgccacaaccacaacccacaaagtgatcttcagcaaggctcctgagagcaccacaactagtcctacactgcctaagcccagcacacctcctggcagctcttgtggaggcatgaaagtgaagcagctggtggacaaggtggaagaactgctgagcaagaactaccacctcgtgaatgaggtggcacggctcgtgaagctcgtgggagaaagaggtggctgatag

SEQ ID NO: 2 - DNA, 2121 bp, Human gammaherpesvirus 4
  source 1..2121: mol_type = genomic DNA
atgcagctgctgtgcgtgttctgcctggtgctgctgtgggaagtgggagctgccagcctgagcgaagtgaagctgcacctggacatcgagggccacgccagccactacaccatcccttggacagagctgatggccaaggtgcctggactgtctcctgaagctctgtggcgagaagccaacgtgaccgaggatctggcttccatgctgaaccggtacaagctgatctacaagaccagcggaaccctgggaatcgctctggcagagcctgtggatatccctgctgtgtctgagggcagcatgcaggtggacgccagcaaagtgcacccaggagtgatcagcggactgaatagtcctgcctgtatgctgagcgctcctctggaaaagcagctgttctactacatcggcaccatgctgcctaacaccagaccccacagctacgtgttctaccagctgcggtgccacctgagctacgtcgctctgagcatcaacggagacaagttccagtacaccggagctatgaccagcaagttcctgatgggtacctacaagagagtgaccgagaagggagacgaacacgtgctgagcctggtgttcggcaagaccaaggacctgcctgacctgagaggacccttcagctaccctagcctgacaagcgctcagagcggagactacagcctcgtgatcgtgaccaccttcgtgcactacgccaacttccacaactacttcgtgcccaacctgaaggacatgttcagcagagccgtgaccatgacagctgccagctacgccagatacgtgctgcagaaactggtgctgctggaaatgaagggaggatgcagagagcctgagctggacaccgagacactgacaaccatgttcgaggtgtccgtggccttcttcaaagtgggacacgctgtgggagagacaggcaatggctgtgtggacctgagatggctggccaagagcttcttcgagctgaccgtgctgaaggatatcattggcatctgctacggagccaccgtgaagggcatgcagagctacggactggaaagactggcagctatgctgatggctaccgtgaagatggaagaactgggacacctcaccacagagaagcaggaatacgctctgagactggccaccgtgggctatcctaaagctggagtgtactccggactgatcggtggagctacaagcgtgctgctgagtgcctacaaccgacaccctctgttccagcctctgcacaccgtgatgagagagacactgttcatcggaagccacgtggtgctgcgagagctgagactgaatgtgaccacacagggacctaacctggctctgtatcagctgctgagcaccgctctgtgtagcgctctggagatcggagaggtgctgagaggactggctctgggcacagagagcggactgttcagcccttgctacctgagcctgagattcgacctgaccagagacaagctgctgtccatggctcctcaggaagccacactggatcaggcagccgtgtccaacgctgtggatggctttctgggacgactgtcactggaaagagaggacagggacgcctggcatctgcctgcctataagtgcgtggaccgactggacaaggtgctgatgatcattcccctgatcaatgtgaccttcatcatcagctccgaccgagaggtgcgaggcagtgccctgtatgaagccagcaccacatacctgagcagcagcctgttcctgagccctgtgatcatgaacaagtgcagccagggagctgtggctggagagcctagacagatccccaagatccagaacttcacccgaacccagaagtcctgcatcttctgtggcttcgctctgctgtcctacgacgagaaagagggactggaaaccaccacctacatcaccagccaggaagtgcagaacagcatcctgtccagcaattacttcgacttcgacaacctgcatgtgcactacctgctgctgaccacaaacggcacagtgatggaaatcgctggactgtacgaggaacgagctcatgtggtgctggccatcatcctgtactttatcgcctttgctcttgggatcttcctggtgcacaagatcgtgatgttcttcctgtga

SEQ ID NO: 3 - DNA, 414 bp, Human gammaherpesvirus 4
  source 1..414: mol_type = genomic DNA
atgagagccgtgggagtgttcctggccatctgcctcgtgaccatcttcgtgctgcccacctggggtaactgggcttacccttgttgccacgtgacccagctgagagcccagcatctgctggcactggagaacatcagcgacatctacctggtgtccaaccagacctgcgacggcttcagcctggcatccctgaacagtcccaagaacggcagcaatcagctcgtgatctccagatgtgccaacggactgaacgtggtgtccttcttcatctccatcctgaagcggagcagcagcgctctgacaggccacctgagagagctgctgaccaccctggaaaccctgtacggcagcttcagcgtggaagatctgttcggagccaacctgaacagatacgcctggcacagaggaggctga

SEQ ID NO: 4 - DNA, 2283 bp, Human gammaherpesvirus 4
  source 1..2283: mol_type = genomic DNA
atgagcctggtgtccgactactgcaacgtgctgaacaaagagttcacagctggcagcgtggaaatcactctgcggagctacaagatctgcaaggccttcatcaacgaggccaaggctcatggcagagaatggggtggactgatggccaccctgaacatctgcaatttctgggctatcctgcggaacaacagagtgagacggagagccgagaacgctggcaatgatgcctgctctatcgcctgtcctatcgtgatgagatacgtgctggaccacctgatcgtcgtgaccgaccggttcttcatccaagctcccagcaatagagtgatgattcctgccaccatcggcacagccatgtacaagctgctgaagcacagtagagtgagagcctacacctacagcaaggtgctgggagtggacagagcagccatcatggctagtggcaaacaggtggtggaacacctgaaccggatggagaaagagggactgctgagcagcaagttcaaggccttctgcaagtgggtgttcacctaccctgtgctggaagagatgttccagaccatggtgtccagcaagacaggacacctgaccgacgacgtgaaagatgtgagagctctgatcaagacactgcccagagccagctacagctctcacgcaggtcagagaagctacgtgtcaggcgtgctgcctgcatgtctgctgtccaccaagagcaaggctgtggaaacacccatcctggtgtctggagccgacagaatggacgaagaactgatgggcaacgacggtggagccagccatacagaggccagatactctgagtctggccagttccacgccttcaccgacgagctggaaagcctgcctagccctaccatgcctctgaaacctggagcccagtctgccgactgtggcgatagctcctcttcaagcagtgacagtggcaacagcgataccgagcagagcgagagagaagaggctagagccgaagctcctagactgagagcacccaagagcagaagaaccagcagacccaacagaggacagacaccctgtccttctaacgctgcagagcctgagcagccttggattgctgccgtgcaccaggaaagcgacgagagacctatcttcccacatcccagcaagccaaccttcctgatgttcgatcctgctcctgaggcaggctctgccatctccgatgtgttcgagggacgggaagtgtgccagcccaagcggatcagacccttccatcctcctggaagcccttgggctaacagacctctgccagcctctcttgctccaacacctacaggacctgtgcacgagcctgtgggcagcctgacaccagctccagtgcctcagcctctggatccagctcctgccgtgacacctgaggccagccatctgctggaagatcccgacgaagagacaagccaggcagtgaaggccctgagagagatggctgatacagtgatcccacagaaagaagaggcagccatttgtggccagatggacctgtctcaccctccacctagaggccacctggatgagctgaccacaaccctggaatccatgaccgaggacctgaacctggacagccctctgactcccgagctgaacgagatcctggacacctttctgaacgacgagtgcctgctgcacgccatgcacatcagcaccggagacagcatcttcgacaccagcctgttcatgatggaccctaacagcaccagcgaggacgtgaagttcactcccgacccttaccaggtgcccttcgtgcaggccttcgatcaggccaccgagaatgcctgcagaagtgcctacaagcaggacgaccagcactacagagagcctgagcctctgcctcagggacagctgacagcctaccacgtgtcacagcctgcacccgagaacgcctaccaggcctatgctgcacctcagctgtttcccgtgtccgacatcacccagaaccaacagaccaaccaggctggaggcgaagctcctcagcctggcgataatagcaccgtgcagacagctgcagctgtggtgtttgcttgccctggagctaatcagggtcagcagctggcagatattggcgtgccacagccagcacctgtggctgctcctgccagaaggaccagaaagcctcagcaacccgagagcctggaagagtgcgacagcgaactggaaatcaagcggagcgagaacgacagactggaactgctgctgaaacagatgtgtcccagcctggacgtggactccatcatccctagaacacccgactgataa

SEQ ID NO: 5 - DNA, 2892 bp, Human gammaherpesvirus 4
  source 1..2892: mol_type = genomic DNA
atggagacagacacactcctgctatgggtactgctgctctgggttccaggttccactggtgacgacaaggacagacctggaccacctgccctggacgacaacatggaagaggaagttcccagcaccagcgtggtgcaggaacaggtgtcagctggcgactgggagaacgtgctgatcgagctgagcgacagcagcagcgagaaagaggctgaggacgcacatctggaacctgctcagaaaggcaccgtggaccatgatgctggaggctctgctccagccagacctatgctgcctcctcagcctgatctgcctggcagagaggccatcctgagaagattcccactggacctgcggaccctgctgcaggctattggagcagctgccacacggatcgacaccagagccatcgaccagttcttcggcagccagatcagcaacaccgagatgtacattatgtacgccatggccatcagacaggccattagggatcaggccaaatggaggctgcagacactggctgcaggctggccgatgggttaccaggcctacagcagctggatgtacagctacaccgaccaccagaccacacccaccttcgtgcatctgcaagcagcagctggagctactggagggagaagatgccacgtgacattcagtgctggcaccttcaagctgcccagatgcacacctggagacagacagtggctgtacgtgcagtctagcgtgggcaacatcgtgcagagctgcaaccctcggtacagcatcttcttcgactacatggccattcaccggtctctgaccaagatctgggaagaggtgctgacaccagaccagagagtgtcctttatggaattcctgggcttcctgcagcggaccgacctgagctacatcaagagcttcgtgtccgacgctctgggcaccaccagcatccagactccctggatcgacgacaaccctagcacagaaacagctcaggcttggaacgcaggcttcctgagaggcagagcctacggcatcgacctgctgagaacagagggagagcatgtggaaggagctaccggtgaaaccagagaggaaagcgaggacaccgagagcgacggagacgacagactgctgctgatgaccgagcaaggcaaagaagtgctggagaaggccagaggctccacctacggcacacctagacctcctgtgcccaagcctagacctgaggtgccacagagcgacgagacagccacatctcacggctctgcccaggtgcctgagccacctacaattcatctggcagctcagggcatggcctacccactgcatgaacagcacggcatggctccttgtcctgtggctcaggcacctcctacacctctgcctcctgtgtctcctggcgatcagctgcctggcgtgttcagcgacggaagagtggcctgtgctcctgttcctgcacctgcaggaccaattgtgagaccttgggagcctagcctgacacaggctgcaggacaggcctttgctccggtgagacctcagcacatgcctgtggaacctgtgccagtgcctaccgttgccctggaaagacccgtgtaccctaagcctgtgaggccagctccacccaagattgccatgcagggacctggtgagacaagtggcatttggaggcctgctccttggacacccaatccacctagaagcccttcgcagatgagcgtgctgagagccgaggcacaagtgaagcaggccagcgtggaagtgcagccacctcagctgactcaggtgtcacctcagcagcccatggagggacctctggtacctgagcagcagatgtttcctggtgctcctttcagccaggtggctgatgtcgtgagggctcctggcgtgccagctatgcagccacagtacttcgacctgcctctgatccagcccatcagccagggagcaccagtggctcctctgagagcctctatgggacctgtgcctccagtgccagcaacccagcctcagtatttcgatatccctctgaccgagcctatcaatcagggagcctctgcagcacacttcctgccacagcagcctatggagggaccactggtgcctgaacaatggatgttcccaggagctgctctgagccagtctgtgagaccaggcgtggcacagagccagtactttgatctgcctctgacacagccaatcaaccacggagcacctgctgctcactttctgcaccaacctccaatggaaggtccttgggtaccagagcagtggatgtttcagggagctcctcctagccagggcaccgatgtggtgcagcatcagctggacgctctgggctacacactgcacggactgaatcatccaggtgtgccagtgtctccagccgttaatcagtaccacctgagccaggctgccttcggcctgcccattgatgaggatgagagcggagagggcagcgacacatctgagccttgcgagatccacggcagaccctgtcctcaggcaccagaatggccagttcaggaagaaggaggccaggacgccaccgagattcacggaaggcctagacccagaactcctgagtggccagtgcagggagagggtggacagaatgtggctggtcctgagactagacgggtggtggtgtctgctgtggtgcacatgtgtcaggacgacgagttccctgacctgcaggatcctcctgatgaggccggagggggtggctctggtgggggagggtccggcggaggcggttcagctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttgataa

SEQ ID NO: 6 - DNA, 34 bp, synthetic construct
  source 1..34: mol_type = other DNA
ataacttcgtataggatactttatacgaagttat

SEQ ID NO: 7 - DNA, 124 bp, synthetic construct
  source 1..124: mol_type = other DNA
taaaaatagaaactataatcatataatagtgtaggttggtagtattgctcttgtgactagagactttagttaaggtactgtaaaaatagaaactataatcatataatagtgtaggttggtagta

SEQ ID NO: 8 - DNA, 37 bp, synthetic construct
  source 1..37: mol_type = other DNA
aaaaattgaaattttattttttttttttggaatataa

SEQ ID NO: 9 - DNA, 108 bp, synthetic construct
  source 1..108: mol_type = other DNA
taaaaattgaaaataaatacaaaggttcttgagggttgtgttaaattgaaagcgagaaataatcataaataatttcattatcgcgatatccgttaagtttgtatcgta

SEQ ID NO: 10 - DNA, 100 bp, synthetic construct
  source 1..100: mol_type = other DNA
tatattattaagtgtggtgtttggtcgatgtaaaatttttgtcgataaaaattaaaaaataacttaatttattattgatctcgtgtgtacaaccgaaatc

SEQ ID NO: 11 - DNA, 60 bp, synthetic construct
  source 1..60: mol_type = other DNA
agcggagagggcagaggaagtctgctaacatgcggtgacgtcgaggagaatcctggacct

SEQ ID NO: 12 - DNA, 66 bp, synthetic construct
  source 1..66: mol_type = other DNA
ggatccggcgccaccaatttctccctgctgaaacaggccggcgatgtggaagagaatccaggccct

SEQ ID NO: 13 - DNA, 147 bp, synthetic construct
  source 1..147: mol_type = other DNA
cctaagcccagcacacctcctggcagctcttgtggaggcatgaaagtgaagcagctggtggacaaggtggaagaactgctgagcaagaactaccacctcgtgaatgaggtggcacggctcgtgaagctcgtgggagaaagaggtggc

SEQ ID NO: 14 - protein, 15 aa, Human gammaherpesvirus 4
MEAALLVCQYTIQSL

SEQ ID NO: 15 - protein, 15 aa, Human gammaherpesvirus 4
LGAGELALTMRSKKL

SEQ ID NO: 16 - protein, 15 aa, Human gammaherpesvirus 4
ELALTMRSKKLPINV

SEQ ID NO: 17 - protein, 15 aa, Human gammaherpesvirus 4
MEAALLVCQYTIQSL

SEQ ID NO: 18 - protein, 15 aa, Human gammaherpesvirus 4
LGAGELALTMRSKKL

SEQ ID NO: 19 - protein, 15 aa, Human gammaherpesvirus 4
ELALTMRSKKLPINV